SEQUENCE LISTING (12 sequences)

SEQ ID NO: 1 - protein, 1273 aa, Unknown
  SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1259 aa, Artificial Sequence
  HKU3 NTD/SARS1 RBD/SARS2 S2 chimera
MFVFLVLLPLVSSQCGIISRKPQPKMAQVSSSRRGVYYNDDIFRSDVLHLTQDYFLPFDSNLTQYFSLNVDSDRYTYFDNPILDFGDGVYFAATEKSNVIRGWIFGSSFDNTTQSAVIVNNSTHIIIRVCNFNLCKEPMYTVSRGTQQNAWVYQSAFNCTYDRVEKSFQLDTTPKTGNFKDLREYVFKNRDGFLSVYQTYTAVNLPRGLPTGFSVLKPILKLPFGINITSYRVVMAMFSQTTSNFLPESAAYYVGNLKYSTFMLRFNENGTITDAVDCSQNPLAELKCTIKNFTVEKGIYQTSNFRVQPTESIVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1256 aa, Artificial Sequence
  SARS2 RBD/SARS1 S1 and S2 chimera
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 4 - protein, 1272 aa, Artificial Sequence
  SARS1 RBD/SARS2 S1 and S2 chimera
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1272 aa, Artificial Sequence
  SCH014 RBD/SARS2 S1 and S2 chimera
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1259 aa, Unknown
  SARS-CoV-1
MKILIFAFLANLAKAQEGCGIISRKPQPKMAQVSSSRRGVYYNDDIFRSDVLHLTQDYFLPFDSNLTQYFSLNVDSDRYTYFDNPILDFGDGVYFAATEKSNVIRGWIFGSSFDNTTQSAVIVNNSTHIIIRVCNFNLCKEPMYTVSRGTQQNAWVYQSAFNCTYDRVEKSFQLDTTPKTGNFKDLREYVFKNRDGFLSVYQTYTAVNLPRGLPTGFSVLKPILKLPFGINITSYRVVMAMFSQTTSNFLPESAAYYVGNLKYSTFMLRFNENGTITDAVDCSQNPLAELKCTIKNFNVDKGIYQTSNFRVSPTQEVIRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLVKNQCVNFNFNGLKGTGVLTSSSKRFQSFQQFGRDTSDFTDSVRDPQTLEILDISPCSFGGVSVITPGTNASSEVAVLYQDVNCTDVPTAIRADQLTPAWRVYSTGVNVFQTQAGCLIGAEHVNASYECDIPIGAGICASYHTASVLRSTGQKSIVAYTMSLGAENSIAYANNSIAIPTNFSISVTTEVMPVSMAKTAVDCTMYICGDSLECSNLLLQYGSFCTQLNRALTGIAIEQDKNTQEVFAQVKQMYKTPAIKDFGGFNFSQILPDPSKPTKRSFIEDLLFNKVTLADAGFMKQYGDCLGDVSARDLICAQKFNGLTVLPPLLTDEMVAAYTAALVSGTATAGWTFGAGAALQIPFAMQMAYRFNGIGVTQNVLYENQKLIANQFNSAIGKIQESLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAYFPREGVFVSNGTSWFITQRNFYSPQLITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 7 - protein, 1242 aa, Unknown
  Bat CoV HKU3
MKILIFAFLANLAKAQEGCGIISRKPQPKMAQVSSSRRGVYYNDDIFRSDVLHLTQDYFLPFDSNLTQYFSLNVDSDRYTYFDNPILDFGDGVYFAATEKSNVIRGWIFGSSFDNTTQSAVIVNNSTHIIIRVCNFNLCKEPMYTVSRGTQQNAWVYQSAFNCTYDRVEKSFQLDTTPKTGNFKDLREYVFKNRDGFLSVYQTYTAVNLPRGLPTGFSVLKPILKLPFGINITSYRVVMAMFSQTTSNFLPESAAYYVGNLKYSTFMLRFNENGTITDAVDCSQNPLAELKCTIKNFNVDKGIYQTSNFRVSPTQEVIRFPNITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKLSTELVKNQCVNFNFNGLKGTGVLTSSSKRFQSFQQFGRDTSDFTDSVRDPQTLEILDISPCSFGGVSVITPGTNASSEVAVLYQDVNCTDVPTAIRADQLTPAWRVYSTGVNVFQTQAGCLIGAEHVNASYECDIPIGAGICASYHTASVLRSTGQKSIVAYTMSLGAENSIAYANNSIAIPTNFSISVTTEVMPVSMAKTAVDCTMYICGDSLECSNLLLQYGSFCTQLNRALTGIAIEQDKNTQEVFAQVKQMYKTPAIKDFGGFNFSQILPDPSKPTKRSFIEDLLFNKVTLADAGFMKQYGDCLGDVSARDLICAQKFNGLTVLPPLLTDEMVAAYTAALVSGTATAGWTFGAGAALQIPFAMQMAYRFNGIGVTQNVLYENQKLIANQFNSAIGKIQESLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTAPAICHEGKAYFPREGVFVSNGTSWFITQRNFYSPQLITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 8 - protein, 1256 aa, Unknown
  Bat CoV SHC014
MKLLVLVFATLVSSYTIEKCLDFDDRTPPANTQFLSSHRGVYYPDDIFRSNVLHLVQDHFLPFDSNVTRFITFGLNFDNPIIPFRDGIYFAATEKSNVIRGWVFGSTMNNKSQSVIIMNNSTNLVIRACNFELCDNPFFVVLKSNNTQIPSYIFNNAFNCTFEYVSKDFNLDLGEKPGNFKDLREFVFRNKDGFLHVYSGYQPISAASGLPTGFNALKPIFKLPLGINITNFRTLLTAFPPRPDYWGTSAAAYFVGYLKPTTFMLKYDENGTITDAVDCSQNPLAELKCSVKSFEIDKGIYQTSNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNTSSEVAVLYQDVNCTDVPVAIHADQLTPSWRVYSTGNNVFQTQAGCLIGAEHVDTSYECDIPIGAGICASYHTVSSLRSTSQKSIVAYTMSLGADSSIAYSNNTIAIPTNFSISITTEVMPVSMAKTSVDCNMYICGDSTECANLLLQYGSFCTQLNRALSGIAVEQDRNTREVFAQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGSCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 9 - protein, 1269 aa, Artificial Sequence
  Chimera 1 HKU3-1 NTD/SARS-COV RBD/SARS-CoV-2 S2
MAISGVPVLGFFIIAVLMSAQESWAGIISRKPQPKMAQVSSSRRGVYYNDDIFRSDVLHLTQDYFLPFDSNLTQYFSLNVDSDRYTYFDNPILDFGDGVYFAATEKSNVIRGWIFGSSFDNTTQSAVIVNNSTHIIIRVCNFNLCKEPMYTVSRGTQQNAWVYQSAFNCTYDRVEKSFQLDTTPKTGNFKDLREYVFKNRDGFLSVYQTYTAVNLPRGLPTGFSVLKPILKLPFGINITSYRVVMAMFSQTTSNFLPESAAYYVGNLKYSTFMLRFNENGTITDAVDCSQNPLAELKCTIKNFTVEKGIYQTSNFRVQPTESIVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1268 aa, Artificial Sequence
  Chimera 2 SARS-CoV-2 RBD/SARS-CoV NTD and S2
MAISGVPVLGFFIIAVLMSAQESWASDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 11 - protein, 1282 aa, Artificial Sequence
  Chimera 3 SARS-CoV RBD/SARS-CoV-2 NTD and S2
MAISGVPVLGFFIIAVLMSAQESWAVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1282 aa, Artificial Sequence
  Chimera 4 RsSHC014 RBD/Remaining Spike SARS-CoV-2
MAISGVPVLGFFIIAVLMSAQESWAVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2024/0018191 A1
(43) Pub. Date: Jan. 18, 2024

(54) CHIMERIC CORONAVIRUS S PROTEIN COMPOSITIONS AND METHODS OF USE

(71) Applicant: THE UNIVERSITY OF NORTH CAROLINA AT CHAPEL HILL, Chapel Hill, NC (US)

(72) Inventors: Ralph S. Baric, Haw River, NC (US); David R. Martinez, Durham, NC (US)

(21) Appl. No.: 18/250,559

(22) PCT Filed: Oct. 27, 2021
(86) PCT No.: PCT/US2021/056752

§ 371 Date: Apr. 26, 2023

Related U.S. Application Data
(60) Provisional application No. 63/106,247, filed on Oct. 27, 2020.

(51) Int. Cl. C07K 14/005; A61K 39/215; A61P 31/14

(52) U.S. Cl. CPC C07K 14/005; A61K 39/215; A61P 31/14; C12N 2770/20022; C12N 2770/20034; C12N 2770/20023; C12N 2770/36123; A61K 2039/5258

(57) ABSTRACT

This invention relates to chimeric coronavirus S proteins and methods of their use, for example, to treat and/or prevent diseases or disorders caused by infection by a coronavirus.

### STATEMENT OF PRIORITY

This application claims the benefit, under 35 U.S.C. § 119(e), of U.S. Provisional Application No. 63/106,247, filed on Oct. 27, 2020, the entire contents of which are incorporated by reference herein.

### STATEMENT OF GOVERNMENT SUPPORT

This invention was made with government support under Grant Numbers AI149644 and AI152296 awarded by the National Institutes of Health. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-885WO_ST25.txt, 132,350 bytes in size, generated on Oct. 21, 2021 and filed via EFS-Web, is provided in lieu of a paper copy. This Sequence Listing is hereby incorporated herein by reference into the specification for its disclosures.

### FIELD OF THE INVENTION

This invention relates to chimeric coronavirus S proteins and methods of their use, for example, to treat and/or prevent diseases or disorders caused by infection of a coronavirus.

### BACKGROUND OF THE INVENTION

Coronaviruses (CoVs) are positive-sense, single-stranded RNA enveloped viruses that belong to the Coronaviridae family in the Nidovirales order. These viruses are found in a wide variety of animals and can cause respiratory and enteric disorders. Coronavirus particles have a helical nucleocapsid enveloped by a lipid bilayer with inserted structural proteins including a Spike (S), Membrane (M), and Envelope (E) proteins, and/or in some CoVs, a Hemagglutinin-Esterase (HE) protein.

In 2003, SARS-CoV-1 infected at least 8,000 individuals and killed about 800. As of June 2020, the SARS-CoV-2 virus that causes COVID-19 has caused over 10 million infections and killed over 500,000 people worldwide.

The S protein (spike protein) of Group 2B coronaviruses is the main target of human antibody responses that can block infection. Group 2B coronaviruses that have spread from their host reservoirs into humans are diverse and distinct from one another (FIG. 1). There is a need for broadly neutralizing vaccines against current and future coronavirus pandemics.

The present invention overcomes shortcomings in the art by providing methods and compositions comprising chimeric coronavirus S proteins for inducing broadly protective immune responses and treating and/or preventing diseases and disorders caused by infection by a coronavirus.

### SUMMARY OF THE INVENTION

A first aspect of the present invention provides a chimeric coronavirus S protein, comprising a coronavirus S protein backbone from a first coronavirus (e.g., a backbone coronavirus) that comprises the following amino acid substitutions wherein the numbering is based on the reference amino acid sequence of SEQ ID NO:1: a) a first region comprising amino acid residues 16-305 comprising a coronavirus S protein N-terminal domain (NTD) from a second coronavirus that is different from the first coronavirus; and/or b) a second region comprising amino acid residues 330-521 comprising a coronavirus S protein receptor binding domain (RBD) of a third coronavirus that is different from the first coronavirus and/or second coronavirus. In some embodiments, the second coronavirus may be a different coronavirus from the third coronavirus. In some embodiments, the second coronavirus may be the same coronavirus as the third coronavirus. In some embodiments, the chimeric coronavirus S protein is derived from a subgroup 2b coronavirus.

In further aspects, the present invention further provides an isolated nucleic acid molecule encoding the chimeric coronavirus spike protein of this invention, as well as vectors, particles, and compositions comprising the chimeric coronavirus S protein and/or the isolated nucleic acid molecule of this invention. Also provided are compositions comprising the chimeric coronavirus S proteins, isolated nucleic acid molecules, particles, and/or vectors of this invention in a pharmaceutically acceptable carrier.

Another aspect of the present invention provides a method of producing an immune response to a coronavirus in a subject, comprising administering to the subject an effective amount of the chimeric coronavirus S protein, nucleic acid molecule, vector, VRP, VLP, coronavirus particle, population and/or composition of the present invention, singly, or in any combination, thereby producing an immune response to a coronavirus in the subject.

Another aspect of the present invention provides a method of treating a coronavirus infection in a subject in need thereof, comprising administering to the subject an effective amount of the chimeric coronavirus S protein, nucleic acid molecule, vector, VRP, VLP, coronavirus particle, population and/or composition of the present invention, singly, or in any combination, thereby treating a coronavirus infection in the subject.

Another aspect of the present invention provides a method of preventing a disease or disorder caused by a coronavirus infection in a subject, comprising administering to the subject an effective amount of the chimeric coronavirus S protein, nucleic acid molecule, vector, VRP, VLP, coronavirus particle, population and/or composition of the present invention, singly, or in any combination, thereby preventing a disease or disorder caused by a coronavirus infection in the subject.

Another aspect of the present invention provides a method of protecting a subject from the effects of coronavirus infection, comprising administering to the subject an effective amount of the chimeric coronavirus S protein, nucleic acid molecule, vector, VRP, VLP, coronavirus particle, population and/or composition of the present invention, singly, or in any combination, thereby protecting the subject from the effects of coronavirus infection.

An additional aspect of the present invention provides a method of identifying a coronavirus S protein for administration to elicit an immune response to coronavirus in a subject, comprising: a) contacting a sample obtained from a subject known to be or suspected of being infected with a coronavirus with a chimeric coronavirus S protein of the present invention under conditions whereby an antigen/antibody complex can form; and b) detecting formation of an antigen/antibody complex, whereby detection of formation of the antigen/antibody complex comprising the chimeric coronavirus S protein identifies the presence in said sample of antibodies that bind an S protein of at least one of the coronaviruses of said chimeric coronavirus S protein (e.g., said first, second, or third coronavirus), thereby identifying a coronavirus S protein for administration to a subject for whom eliciting an immune response to a coronavirus is needed or desired.

A further aspect of the present invention provides a method of detecting an antibody that binds a coronavirus S protein in a sample, comprising: a) contacting the sample with the coronavirus S protein under conditions whereby an antigen/antibody complex can form; and b) detecting the formation of an antigen/antibody complex, thereby detecting the presence in the sample of an antibody that binds a coronavirus S protein.

These and other aspects of the invention are set forth in more detail in the description of the invention below.

### BRIEF DESCRIPTION OF THE DRAWINGS

FIG. 1 shows a schematic of the phylogenetic relationships of Group 2B coronaviruses.

FIG. 2 shows a sequence alignment of the spike proteins of HKU3 (SEQ ID NO:7), SARS-CoV-1 (SEQ ID NO:6), SARS-CoV-2 (SEQ ID NO:1), and SCH014 (SEQ ID NO:8).

FIGS. 3A-3C show the design of chimeric Sarbecovirus spike vaccines. FIG. 3A shows a diagram of genetic diversity of pandemic and bat zoonotic coronaviruses. SARS-CoV is shown in light blue, RsSHC014 is shown in purple, and SARS-CoV-2 is shown in red. FIG. 3B shows a schematic of the spike chimeras, wherein Spike chimera 1 includes the NTD from HKU3-1, the RBD from SARS-CoV, and the rest of the spike from SARS-CoV-2; Spike chimera 2 includes the RBD from SARS-CoV-2 and the NTD and S2 from SARS-CoV; Spike chimera 3 includes the RBD from SARS-CoV and the NTD and S2 SARS-CoV-2; and Spike chimera 4 includes the RBD from RsSHC014 and the rest of the spike from SARS-CoV-2. SARS-CoV-2 furin KO spike vaccine and is the norovirus capsid vaccine. FIG. 3C shows a table summary of chimeric spike constructs.

FIGS. 4A-4J show data plots of human pathogenic coronavirus spike binding and hACE2-blocking responses in chimeric and monovalent SARS-CoV-2 spike-vaccinated mice. Groups shown in FIGS. 4A-4J include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). Serum antibody ELISA binding responses were measured in the five different vaccination groups. Pre-immunization, post prime, and post-boost binding responses were evaluated against Sarbecoviruses, MERS-CoV, and common-cold CoV antigens including: (FIG. 4A) SARS-CoV Toronto Canada (Tor2) S2P, (FIG. 4B) SARS-CoV-2 S2P D614G, (FIG. 4C) SARS-CoV-2 RBD, (FIG. 4D) SARS-CoV-2 NTD, (FIG. 4E) Pangolin GXP4L spike, (FIG. 4F) RaTG13 spike, (FIG. 4G) RsSHC014 S2P spike, (FIG. 4H) HKU3-1 spike, (FIG. 4I) MERS-CoV spike, (FIG. 4J) hACE2 blocking responses against SARS-CoV-2 spike in the distinct immunization groups. Statistical significance for the binding and blocking responses is reported from a Kruskal-Wallis test after Dunnett's multiple comparison correction. *p<0.05, **p<0.01, ***p<0.001, and ****p<0.0001.

FIGS. 5A-5F show data plots of live Sarbecovirus neutralizing antibody responses in vaccinated mice. Groups shown in FIGS. 5A-5F include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). Neutralizing antibody responses in mice from the five different vaccination groups were measured using nanoluciferase-expressing recombinant viruses. FIG. 5A shows a data plot of SARS-CoV neutralizing antibody responses from baseline and post boost in the distinct vaccine groups. FIG. 5B shows a data plot of SARS-CoV-2 neutralizing antibody responses from baseline and post boost. FIG. 5C shows a data plot of RsSHC014 neutralizing antibody responses from baseline and post boost. FIG. 5D shows a data plot of WIV-1 neutralizing antibody responses from baseline and post boost. FIG. 5E shows a data plot of the neutralization activity in groups 1 and 4 against SARS-CoV-2 D614G, South African B.1.351, U.K. B.1.1.7, and mink cluster 5 variant. FIG. 5F shows a data plot of neutralization comparison of SARS-CoV-2 D614G vs. South African B.1.351, vs. U.K. B1.1.7, and mink cluster 5 variant. Statistical significance for the live-virus neutralizing antibody responses is reported from a Kruskal-Wallis test after Dunnett's multiple comparison correction. *p<0.05, **p<0.01, ***p<0.001, and ****p<0.0001.

FIGS. 6A-6L show data plots of in vivo protection against Sarbecovirus challenge after mRNA-LNP vaccination. Groups shown in FIGS. 6A-6L include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). FIG. 6A shows a data plot of percent starting weight from the different vaccine groups of mice challenged with SARS-CoV MA15. FIG. 6B shows a data plot of SARS-CoV MA15 lung viral titers in mice from the distinct vaccine groups. FIG. 6C shows a data plot of SARS-CoV MA15 nasal turbinate titers. FIG. 6D shows a data plot of percent starting weight from the different vaccine groups of mice challenged with SARS-CoV-2 MA10. FIG. 6E shows a data plot of SARS-CoV-2 MA10 lung viral titers in mice from the distinct vaccine groups. FIG. 6F shows a data plot of SARS-CoV-2 MA10 nasal turbinate titers.

FIG. 6G shows a data plot of percent starting weight from the different vaccine groups of mice challenged with WIV-1. FIG. 6H shows a data plot of WIV-1 lung viral titers in mice from the distinct vaccine groups. FIG. 6I shows a data plot of WIV-1 nasal turbinate titers.

FIG. 6J shows a data plot of percent starting weight from the different vaccine groups of mice challenged with SARS-CoV-2 B.1.351. FIG. 6K shows a data plot of SARS-CoV-2 B.1.351 lung viral titers in mice from the distinct vaccine groups. FIG. 6L shows a data plot of SARS-CoV-2 B.1.351 nasal turbinate titers. Figure legend at the bottom right depicts the vaccines utilized in the different groups. Statistical significance for weight loss is reported from a two-way ANOVA after Dunnett's multiple comparison correction. For lung and nasal turbinate titers, statistical significance is reported from a one-way ANOVA after Tukey's multiple comparison correction. *p<0.05, **p<0.01, ***p<0.001, and ****p<0.0001.

FIGS. 7A-7D show histology images and data plots of lung pathology in vaccinated mice after SARS-CoV and SARS-CoV-2 challenge. Groups shown in FIGS. 7A-7D include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). FIG. 7A shows histology images of hematoxylin and eosin 4 days post infection lung analysis of SARS-CoV MA15 challenged mice from the different groups: group 1: chimeras 1-4 prime and boost, group 2: chimeras 1-2 prime and 3-4, group 3: chimera 4 prime and boost, SARS-CoV-2 furin KO prime and boost, and norovirus capsid prime and boost. FIG. 7B shows data plots of lung pathology quantitation in SARS-CoV MA15 challenged mice from the different groups. Macroscopic lung discoloration score, microscopic acute lung injury (ALI) score, and diffuse alveolar damage (DAD) in day 4 post infection lung tissues are shown. FIG. 7C shows histology images of hematoxylin and eosin 4 days post infection lung analysis of SARS-CoV-2 MA10 challenged mice from the different groups. FIG. 7D shows data plots of lung pathology measurements in SARS-CoV-2 MA10 challenged mice from the different groups. Macroscopic lung discoloration score, microscopic acute lung injury (ALI) score, and diffuse alveolar damage (DAD) in day 4 post infection lung tissues are shown. Statistical significance is reported from a one-way ANOVA after Dunnett's multiple comparison correction. *p<0.05, **p<0.01, ***p<0.001, and ****p<0.0001.

FIGS. 8A-8C show a table, a blot and a data graph of chimeric and wild type spike Sarbecovirus constructs. FIG. 8A provides a table identifying the mouse vaccination strategy using mRNA-LNPs: group 1 received chimeric spike protein 1, 2, 3, and 4 as the prime and boost; group 2 received chimeric spike protein 1 and 2 as the prime and chimeric spike proteins 3 and 4 as the boost; group 3 received chimeric spike protein 4 as the prime and boost; group 5 received the SARS-CoV-2 furin KO prime and boost; and group 5 received a norovirus capsid prime and boost. Different vaccine groups were separately challenged with 1) SARS-CoV MA15, 2) SARS-CoV-2 MA10, 3) RsSHC014 full-length virus, 4) RsSHC014-MA14, 5) WIV-1, and 6) SARS-CoV-2 B.1.351 MA10. FIG. 8B shows an image of a blot showing protein expression of chimeric spikes, SARS-CoV-2 furin KO, and norovirus mRNA vaccines. The extra band between 100 and 150 kDa corresponds to S1.

GAPDH was used as the loading control. FIG. 8C shows a data plot of nanoluciferase expression of RsSHC014/SARS-CoV-2 chimeric spike live viruses.

FIGS. 9A-9D show data plots of human common cold CoV ELISA binding responses in chimeric and monovalent SARS-CoV-2 spike mRNA-LNP-vaccinated mice. Groups shown in FIGS. 9A-9D include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). Pre-immunization, post prime, and post boost binding to (FIG. 9A) HCoV-HKU1 spike protein, (FIG. 9B) HCoV-OC43 spike protein, (FIG. 9C) HCoV-229E spike protein, and (FIG. 9D) HCoV-NL63 spike protein. Statistical significance for the binding and blocking responses is reported from a Kruskal-Wallis test after Dunnett's multiple comparison correction. *p<0.05, **p<0.01, ***p<0.001, and ****p<0.0001.

FIGS. 10A-10H show data plots of comparisons of neutralizing antibody activity of CoV mRNA-LNP vaccines against Sarbecoviruses. FIGS. 10A-10B shows a data plot of group 1 (FIG. 10A) neutralizing antibody responses against SARS-CoV-2, SARS-CoV, RsSHC014, and WIV-1, and (FIG. 10B) fold-change of SARS-CoV, RsSHC014, and WIV-1 neutralizing antibodies relative to SARS-CoV-2. Also shown are Group 2 neutralizing antibody responses (FIG. 10C) against SARS-CoV-2, SARS-CoV, RsSHC014, and WIV-1, and fold-change (FIG. 10D) of SARS-CoV, RsSHC014, and WIV-1 neutralizing antibodies relative to SARS-CoV-2; Group 3 neutralizing antibody responses (FIG. 10E) against SARS-CoV-2, SARS-CoV, RsSHC014, and WIV-1, and fold-change (FIG. 10F) of SARS-CoV, RsSHC014, and WIV-1 neutralizing antibodies relative to SARS-CoV-2; and Group 4 neutralizing antibody responses (FIG. 10G) against SARS-CoV-2, SARS-CoV, RsSHC014, and WIV-1, and fold-change (FIG. 10H) of SARS-CoV, RsSHC014, and WIV-1 neutralizing antibodies relative to SARS-CoV-2.

FIGS. 11A-11F show data plots of in vivo protection assay against Bt-CoV challenge by chimeric spike mRNA-vaccines. Groups shown in FIGS. 11A-11F include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). FIG. 11A shows a data plot of percent starting weight from the different vaccine groups of mice challenged with full-length RsSHC014. FIG. 11B shows a data plot of RsSHC014 lung viral titers in mice from the distinct vaccine groups. FIG. 11C shows a data plot of RsSHC014 nasal turbinate titers in mice from the different immunization groups. FIG. 11D shows a data plot of percent starting weight from the different vaccine groups of mice challenged with RsSHC014-MA15. FIG. 11E shows a data plot of RsSHC014-MA15 lung viral titers in mice from the distinct vaccine groups. FIG. 11F shows a data plot of RsSHC014-MA15nasal turbinate titers in mice from the different immunization groups. Statistical significance is reported from a one-way ANOVA after Tukey's multiple comparison correction. *p<0.05, **p<0.01, ***p<0.001, and ****p<0.0001.

FIGS. 12A-12D show data plots survival analyses of immunized mice challenged with Sarbecoviruses. Groups shown in FIGS. 12A-12D include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). Analysis shown at day 4 post infection from immunized mice infected with SARS-CoV MA15 (FIG. 12A), day 4 post infection from immunized mice infected with SARS-CoV-2 MA10 (FIG. 12B), day 7 post infection from immunized mice infected with SARS-CoV-2 MA10 (FIG. 12C), and day 7 post infection from immunized mice infected with RsSHC014-MA15. Statistical significance is reported from a Mantel-Cox test.

FIGS. 13A-13E show images of histology indicating detection of eosinophilic infiltrates in SARS-CoV MA15 challenged mice. Groups shown in FIGS. 13A-13E include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). FIG. 13A depicts Group 1, showing rare scattered individual eosinophils in the interstitium with some small perivascular cuffs that lack eosinophils. FIG. 13B depicts Group 2, showing bronchiolar cuffs of leukocytes with rare eosinophils. FIG. 13C depicts Group 3, showing hyperplastic bronchus-associated lymphoid tissue (BALT) with rare eosinophils. FIG. 13D depicts Group 4, showing frequent perivascular cuffs that contain eosinophils. FIG. 13E depicts Group 5, showing frequent eosinophils in perivascular cuffs.

FIGS. 14A-14B show data plots of lung cytokine analyses in Sarbecovirus-challenged mice. Groups shown in FIGS. 14A-14B include group 1 (chimeras 1-4 prime/boost), group 2 (chimeras 1-2 prime and 3-4 boost), group 3 (chimera 4 prime/boost), group 4 (SARS-CoV-2 spike furin KO prime/boost), and group 5 (Norovirus capsid prime/boost). FIG. 14A shows analysis of CCL2, IL-1α, G-CSF, and CCL4 in SARS-CoV-infected mice. FIG. 14B shows the same in SARS-CoV-2-infected mice. Statistical significance for the binding and blocking responses is reported from a Kruskal-Wallis test after Dunnett's multiple comparison correction. *p<0.05, **p<0.01, ***p<0.001, and ****p<0.0001.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise. For example, “a” cell can mean one cell or a plurality of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol. 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” nucleic acid or nucleotide sequence (e.g., an “isolated DNA” or an “isolated RNA”) means a nucleic acid or nucleotide sequence separated or substantially free from at least some of the other components of the naturally occurring organism or virus, for example, the cell or viral structural components or other polypeptides or nucleic acids commonly found associated with the nucleic acid or nucleotide sequence.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

Furthermore, an “isolated” cell is a cell that has been partially or completely separated from other components with which it is normally associated in nature. For example, an isolated cell can be a cell in culture medium and/or a cell in a pharmaceutically acceptable carrier.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or modified nucleotide bases. The term “nucleotide sequence” or “nucleic acid sequence” refers to both the sense and antisense strands of a nucleic acid as either individual single strands or in the duplex.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art. Thus, all or a portion of the nucleic acids of the present codons may be synthesized using codons preferred by a selected host. Species-preferred codons may be determined, for example, from the codons used most frequently in the proteins expressed in a particular host species. Other modifications of the nucleotide sequences may result in mutants having slightly altered activity.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

As used herein, by “isolate” or “purify” (or grammatical equivalents) a vector, it is meant that the vector is at least partially separated from at least some of the other components in the starting material.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold, and/or at least about 5%, 10%, 15%, 20%, 25%, 30%, 35%, 40%, 45%, 50%, 55%, 60%, 65%, 70%, 75%, 80%, 85%, 90%, 95%, or 100% or more, or any value or range therein.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts may be referred to as “transcription products” and encoded polypeptides may be referred to as “translation products.” Transcripts and encoded polypeptides may be collectively referred to as “gene products.” If the polynucleotide is derived from genomic DNA, expression may include splicing of the mRNA in a eukaryotic cell. The expression product itself, e.g., the resulting nucleic acid or protein, may also be said to be “expressed.” An expression product can be characterized as intracellular, extracellular, or secreted. The term “intracellular” means something that is inside a cell. The term “extracellular” means something that is outside a cell. A substance is “secreted” by a cell if it appears in significant measure outside the cell, from somewhere on or inside the cell.

The terms “amino acid sequence,” “polypeptide,” “peptide” and “protein” may be used interchangeably to refer to polymers of amino acids of any length. The terms “nucleic acid,” “nucleic acid sequence,” and “polynucleotide” may be used interchangeably to refer to polymers of nucleotides of any length. As used herein, the terms “nucleotide sequence,” “polynucleotide,” “nucleic acid sequence,” “nucleic acid molecule” and “nucleic acid fragment” refer to a polymer of RNA, DNA, or RNA and DNA that is single- or double-stranded, optionally containing synthetic, non-natural and/or altered nucleotide bases.

As used herein, the terms “gene of interest,” “nucleic acid of interest” and/or “protein of interest” refer to that gene/nucleic acid/protein desired under specific contextual conditions.

As used herein, the term “chimera,” “chimeric,” and/or “fusion protein” refer to an amino acid sequence (e.g., polypeptide) generated non-naturally by deliberate human design comprising, among other components, an amino acid sequence of a protein of interest and/or a modified variant and/or active fragment thereof (a “backbone”), wherein the protein of interest comprises modifications (e.g., substitutions such as singular residues and/or contiguous regions of amino acid residues) from different wild type reference sequences (chimera), optionally linked to other amino acid segments (fusion protein). The different components of the designed protein may provide differing and/or combinatorial function.

Structural and functional components of the designed protein may be incorporated from differing and/or a plurality of source material. The designed protein may be delivered exogenously to a subject, wherein it would be exogenous in comparison to a corresponding endogenous protein.

As used herein with respect to nucleic acids, the term “operably linked” refers to a functional linkage between two or more nucleic acids. For example, a promoter sequence may be described as being “operably linked” to a heterologous nucleic acid sequence because the promoter sequence initiates and/or mediates transcription of the heterologous nucleic acid sequence. In some embodiments, the operably linked nucleic acid sequences are contiguous and/or are in the same reading frame.

By the term “treat,” “treating,” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder.

As used herein, the term “prevent,” “prevents,” or “prevention” (and grammatical equivalents thereof) refers to a delay in the onset of a disease or disorder or the lessening of symptoms upon onset of the disease or disorder. The terms are not meant to imply complete abolition of disease and encompass any type of prophylactic treatment that reduces the incidence of the condition or delays the onset and/or progression of the condition.

As used herein, “effective amount” or “therapeutic amount” refers to an amount of a population or composition or formulation of this invention that is sufficient to produce a desired effect, which can be a therapeutic effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutic amount in any individual case can be determined by one of ordinary skill in the art by reference to the pertinent texts and literature and/or by using routine experimentation. (See, for example, Remington, The Science and Practice of Pharmacy (20th ed. 2000)).

An “immunogenic amount” is an amount of the compositions of this invention that is sufficient to elicit, induce and/or enhance an immune response in a subject to which the composition is administered or delivered.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The term “administering” or “administration” of a composition of the present invention to a subject includes any route of introducing or delivering to a subject a compound to perform its intended function (e.g., for use as a vaccine antigen). Administration includes self-administration and the administration by another.

As used herein, the term “antigen” refers to a molecule capable of inducing the production of immunoglobulins (e.g., antibodies). The term “immunogen” can be used interchangeably with “antigen” under certain conditions, e.g., when the antigen is capable of inducing a multi-faceted humoral and/or cellular-mediated immune response. A molecule capable of antibody and/or immune response stimulation may be referred to as antigenic/immunogenic, and can be said to have the ability of antigenicity/immunogenicity. The binding site for an antibody within an antigen and/or immunogen may be referred to as an epitope (e.g., an antigenic epitope). The term “vaccine antigen” as used herein refers to such an antigen/immunogen as used as a vaccine, e.g., a prophylactic, preventative, and/or therapeutic vaccine.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the terms “prime boost immunization,” “prime boost administration,” or “prime and booster” refer to an administration (e.g., immunization) regimen that comprises administering to a subject a primary/initial (priming) administration (e.g., of one or more chimeric coronavirus S protein of the present invention) and at least one secondary (boosting) administration. In some embodiments, the priming administration and the at least one boosting administration may comprise the same composition, administered in multiple (one or more) repetitions. In some embodiments, the priming administration and the at least one boosting administration may comprise different types of compositions, such as different types of chimeric coronavirus S proteins of the present invention.

As used herein, the terms “prime immunization,” “priming immunization,” “primary immunization” or “prime” refer to primary antigen stimulation by using a chimeric coronavirus S protein according to the instant invention.

The term “boost immunization,” “boosting immunization,” “secondary immunization”, or “boost” refers to additional administration (e.g., immunization) of a chimeric coronavirus S protein of the present invention administered to a subject after a primary administration. In some embodiments, the boost immunization may be administered at a dose higher than, lower than, and/or equal to the dose administered as a primary immunization, e.g., when the boost immunization is administered alone without priming.

The prime and boost vaccine compositions may be administered via the same route or they may be administered via different routes. The boost vaccine composition may be administered one or several times at the same or different dosages. It is within the ability of one of ordinary skill in the art to optimize prime-boost combinations, including optimization of the timing and dose of vaccine administration.

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human. In some embodiments, a bird may include, but is not limited to, a chicken, a duck, a turkey, a goose, a quail, a pheasant, a parakeet, a parrot, a macaw, a cockatoo, or a canary.

A “subject in need” of the methods of the invention can be any subject known to have a coronavirus infection and/or an illness to which inhibition of coronavirus infection may provide beneficial health effects, or a subject having an increased risk of developing the same).

A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art.

“Nidovirus” as used herein refers to viruses within the order Nidovirales, including the families Coronaviridae and Arteriviridae. All viruses within the order Nidovirales share the unique feature of synthesizing a nested set of multiple subgenomic mRNAs. See M. Lai and K. Holmes, Coronaviridae: The Viruses and Their Replication, in Fields Virology, pg. 1163, (4^{th} Ed. 2001). Particular examples of Coronaviridae include, but are not limited to, toroviruses and coronaviruses.

“Coronavirus” as used herein refers to a genus in the family Coronaviridae, which family is in turn classified within the order Nidovirales. The coronaviruses are large, enveloped, positive-stranded RNA viruses. They have the largest genome of all RNA viruses and replicate by a unique mechanism that results in a high frequency of recombination. The coronaviruses include antigenic groups I, II, and III. Nonlimiting examples of coronaviruses include SARS coronavirus (SARS-CoV, also known as SARS-CoV-1), SARS-CoV-2 (also known as 2019 novel coronavirus (2019-nCoV) or human coronavirus 2019 (HCoV-19 or hCoV-19), MERS coronavirus, transmissible gastroenteritis virus (TGEV), human respiratory coronavirus, porcine respiratory coronavirus, canine coronavirus, feline enteric coronavirus, feline infectious peritonitis virus, rabbit coronavirus, murine hepatitis virus, sialodacryoadenitis virus, porcine hemagglutinating encephalomyelitis virus, bovine coronavirus, avian infectious bronchitis virus, and turkey coronavirus, as well as chimeras of any of the foregoing. See Lai and Holmes “Coronaviridae: The Viruses and Their Replication” in Fields Virology, (4^{th} Ed. 2001).

A “nidovirus permissive cell” or “coronavirus permissive cell” as used herein can be any cell in which a coronavirus can at least replicate, including both naturally occurring and recombinant cells. In some embodiments the permissive cell is also one that the nidovirus or coronavirus can infect. The permissive cell can be one that has been modified by recombinant means to produce a cell surface receptor for the nidovirus or coronavirus.

### Compositions

The present invention relates to the design of a chimeric coronavirus S protein (also referred to as a spike protein and/or surface protein). The viral S protein is involved in viral attachment, fusion, and entry and is a predominant target for host neutralizing antibodies (the infected host; e.g., an infected human). The S protein comprises, among other domains, the receptor binding domain (RBD), which is the viral domain that binds to human and/or bat ACE2 receptor during entry of the virus into a host (e.g., a human) cell. Other antigenic domains comprised within the S protein that are targets for host neutralizing antibodies include, but are not limited to, the N-terminal domain (NTD).

The chimeric coronavirus S proteins of the present invention may improve protective efficacy of coronavirus vaccines against both zoonotic and pandemic coronaviruses that have the potential to emerge or that have previously emerged in humans. While not wishing to be bound to theory, prophylactic and/or therapeutic vaccination using the chimeric coronavirus S proteins of the present invention may provide the recipient with better protection against diverse coronaviruses compared to a recipient receiving a monomorphic S-protein comprising vaccination, through the elicitation of broadly neutralizing antibodies capable of targeting and neutralizing multiple coronaviruses (e.g., each of the first, second, and/or third coronaviruses of the present invention).

The inventors of the present invention formulated chimeric S proteins and vaccines comprising the same that specifically target distant coronavirus Sarbecovirus strains, including mRNA-based lipid nanoparticle (LNP) vaccines. The chimeric spike vaccines disclosed herein provide an advantage of breadth of protection against multiclade Sarbecoviruses and SARS-CoV-2 variants as compared to a monovalent SARS-CoV-2 vaccine, as the chimeric S protein-based vaccines disclosed herein achieve broad protection and are portable to other high-risk emerging coronaviruses like group 2C MERS-CoV-related strains.

Accordingly, the present invention provides a chimeric coronavirus S protein comprising a coronavirus S protein backbone from a first coronavirus, and one or more regions of amino acid substitutions from one or more other coronavirus that is different from the first coronavirus. This invention additionally relates to the use of the chimeras of the present invention in various methods, such as to produce an immune response, treat a coronavirus infection, prevent a disease or disorder associated with a coronavirus infection and/or caused by a coronavirus infection, protect a subject from the effects of a coronavirus infection, among others. The present invention provides chimeric coronavirus S proteins as well as nucleic acid molecules, vectors, particles, populations, and compositions comprising the same, and methods of using the same.

Thus, one aspect of the invention relates to a chimeric coronavirus S protein, comprising a coronavirus S protein backbone from a first coronavirus (e.g., a backbone coronavirus) that comprises the following amino acid substitutions wherein the numbering is based on the reference amino acid sequence of SEQ ID NO:1: a) a first region comprising amino acid residues 16-305 comprising a coronavirus S protein N-terminal domain (NTD) from a second coronavirus that is different from the first coronavirus; and/or b) a second region comprising amino acid residues 330-521 comprising a coronavirus S protein receptor binding domain (RBD) of a third coronavirus that is different from the first coronavirus and/or second coronavirus.

SEQ ID NO: 1. SARS-COV-2 Sprotein
(NCBI Accession No. MN908947)
MFVFLVLLPLVSSQCVNLTTRTQLPPAYTNSFTRGVYYPDKVERSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTELLKYNENGTITDAVDCALD
PLSETKCTLKSFTVEKGIYQTSNERVQPTESIVRFPNITNLCPFGEVEN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGENCYF
PLQSYGFQPTNGVGYQPYRVVVLSFELLHAPATVCGPKKSTNLVKNKCV
NFNFNGLTGTGVLTESNKKELPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLENKVTLADAGF
IKQYGDCLGDIAARDLICAQKFNGLTVLPPLLTDEMIAQYTSALLAGTI
TSGWTFGAGAALQIPFAMQMAYRENGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

The coronaviruses comprised in the chimeric coronavirus S protein of the present invention may be two or three different coronaviruses. In some embodiments, the first coronavirus is the same as the second coronavirus and/or the third coronavirus. In some embodiments, the first coronavirus is different from the second coronavirus and/or the third coronavirus. In some embodiments, the second coronavirus is the same as the first coronavirus and/or the third coronavirus. In some embodiments, the second coronavirus is different from the first coronavirus and/or the third coronavirus. In some embodiments, the third coronavirus is the same as the first coronavirus and/or the second coronavirus. In some embodiments, the third coronavirus is different from the first coronavirus and/or the second coronavirus.

The chimeric coronavirus S protein of this invention may be derived from (e.g., comprise the backbone of and/or substitutions from) any coronavirus type, including but not limited to, a subgroup 1a coronavirus, a subgroup 1b coronavirus, a subgroup 2a coronavirus, a subgroup 2b coronavirus, a subgroup 2c coronavirus, a subgroup 2d coronavirus and/or a subgroup 3 coronavirus. In some embodiments, the chimeric coronavirus S protein is derived from a subgroup 2b coronavirus.

Nonlimiting examples of subgroup 2b coronaviruses that can be used to produce the chimeric coronavirus spike protein of this invention (e.g., said first coronavirus, second coronavirus and/or third coronavirus) include Bat SARS CoV (GenBank Accession No. FJ211859), SARS CoV (GenBank Accession No. FJ211860), BtSARS.HKU3.1 (GenBank Accession No. DQ022305), BtSARS.HKU3.2 (GenBank Accession No. DQ084199), BtSARS.HKU3.3 (GenBank Accession No. DQ084200), BtSARS.Rm1 (GenBank Accession No. DQ412043), BtCoV.279.2005 (GenBank Accession No. DQ648857), BtSARS.Rf1 (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCoV.CUHK-W1 (GenBank Accession No. AY278554), SARSCoV.GD01 (GenBank Accession No. AY278489), SARSCoV.HC.SZ.61.03 (GenBank Accession No. AY515512), SARSCoV.SZ16 (GenBank Accession No. AY304488), SARSCoV.Urbani (GenBank Accession No. AY278741), SARSCoV.civet010 (GenBank Accession No. AY572035), and SARSCoV.MA.15 (GenBank Accession No. DQ497008), Rs SHC014 (GenBank® Accession No. KC881005), Rs3367 (GenBank® Accession No. KC881006), WiVI S (GenBank® Accession No. KC881007), SARS CoV2 (GenBank Accession No. MN908947), as well as any other subgroup 2b coronavirus now known (e.g., as can be found in the GenBank® Database) or later identified, and any combination thereof.

Nonlimiting examples of subgroup 2c coronaviruses that can be used to produce the chimeric coronavirus spike protein of this invention (e.g., said first coronavirus, second coronavirus and/or third coronavirus) include: Middle East respiratory syndrome coronavirus isolate Riyadh_2_2012 (GenBank Accession No. KF600652.1), Middle East respiratory syndrome coronavirus isolate Al-Hasa_18_2013 (GenBank Accession No. KF600651.1), Middle East respiratory syndrome coronavirus isolate Al-Hasa_17_2013 (GenBank Accession No. KF600647.1), Middle East respiratory syndrome coronavirus isolate Al-Hasa_15_2013 (GenBank Accession No. KF600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_2013 (GenBank Accession No. KF600632.), Middle East respiratory syndrome coronavirus isolate Buraidah_1_2013 (GenBank Accession No. KF600630.1), Middle East respiratory syndrome coronavirus isolate Hafr-Al-Batin_1_2013 (GenBank Accession No. KF600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate Riyadh_1_2012 (GenBank Accession No. KF600612.1), Middle East respiratory syndrome coronavirus isolate Al-Hasa_3_2013 (GenBank Accession No. KF186565.1), Middle East respiratory syndrome coronavirus isolate Al-Hasa_1_2013 (GenBank Accession No. KF186567.1), Middle East respiratory syndrome coronavirus isolate Al-Hasa_2_2013 (GenBank Accession No. KF186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-CoV_SA-N1 (GenBank Accession No. KC667074), following isolates of Middle East Respiratory Syndrome Coronavirus (GenBank Accession No: KF600656.1, GenBank Accession No: KF600655.1, GenBank Accession No: KF600654.1, GenBank Accession No: KF600649.1, GenBank Accession No: KF600648.1, GenBank Accession No: KF600646.1, GenBank Accession No: KF600643.1, GenBank Accession No: KF600642.1, GenBank Accession No: KF600640.1, GenBank Accession No: KF600639.1, GenBank Accession No: KF600638.1, GenBank Accession No: KF600637.1, GenBank Accession No: KF600636.1, GenBank Accession No: KF600635.1, GenBank Accession No: KF600631.1, GenBank Accession No: KF600626.1, GenBank Accession No: KF600625.1, GenBank Accession No: KF600624.1, GenBank Accession No: KF600623.1, GenBank Accession No: KF600622.1, GenBank Accession No: KF600621.1, GenBank Accession No: KF600619.1, GenBank Accession No: KF600618.1, GenBank Accession No: KF600616.1, GenBank Accession No: KF600615.1, GenBank Accession No: KF600614.1, GenBank Accession No: KF600641.1, GenBank Accession No: KF600633.1, GenBank Accession No: KF600629.1, GenBank Accession No: KF600617.1), Coronavirus Neoromicia/PML-PHE1/RSA/2011 GenBank Accession: KC869678.2, Bat Coronavirus Taper/CII_KSA_287/Bisha/Saudi Arabia/GenBank Accession No: KF493885.1,Bat coronavirus Rhhar/CII_KSA_003/Bisha/Saudi Arabia/2013 GenBank Accession No: KF493888.1, Bat coronavirus Pikuh/CII_KSA_001/Riyadh/Saudi Arabia/2013 GenBank Accession No :KF493887.1, Bat coronavirus Rhhar/CII_KSA_002/Bisha/Saudi Arabia/2013 GenBank Accession No: KF493886.1, Bat Coronavirus Rhhar/CII KSA 004/Bisha/Saudi Arabia/2013 GenBank Accession No: KF493884.1, BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV133.2005 (GenBank Accession No. NC_008315), BtCoV.HKU5.5 (GenBank Accession No. EF065512); BtCoV.HKU5.1 (GenBank Accession No. NC_009020), BtCoV.HKU5.2 (GenBank Accession No. EF065510), BtCoV.HKU5.3 (GenBank Accession No. EF065511), human betacoronavirus 2c Jordan-N3/2012 (GenBank Accession No. KC776174.1; human betacoronavirus 2c EMC/2012 (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: KC522086.1, GenBank Accession No: KC522085.1, GenBank Accession No: KC522084.1, GenBank Accession No:KC522083.1, GenBank Accession No: KC522082.1, GenBank Accession No: KC522081.1, GenBank Accession No: KC522080.1, GenBank Accession No: KC522079.1, GenBank Accession No: KC522078.1, GenBank Accession No: KC522077.1, GenBank Accession No: KC522076.1, GenBank Accession No: KC522075.1, GenBank Accession No: KC522104.1, GenBank Accession No: KC522104.1, GenBank Accession No: KC522103.1, GenBank Accession No: KC522102.1, GenBank Accession No: KC522101.1, GenBank Accession No: KC522100.1, GenBank Accession No: KC522099.1, GenBank Accession No: KC522098.1, GenBank Accession No: KC522097.1, GenBank Accession No: KC522096.1, GenBank Accession No: KC522095.1, GenBank Accession No: KC522094.1, GenBank Accession No: KC522093.1, GenBank Accession No: KC522092.1, GenBank Accession No: KC522091.1, GenBank Accession No: KC522090.1, GenBank Accession No: KC522119.1 GenBank Accession No: KC522118.1 GenBank Accession No: KC522117.1 GenBank Accession No: KC522116.1 GenBank Accession No: KC522115.1 GenBank Accession No: KC522114.1 GenBank Accession No: KC522113.1 GenBank Accession No: KC522112.1 GenBank Accession No: KC522111.1 GenBank Accession No: KC522110.1 GenBank Accession No: KC522109.1 GenBank Accession No: KC522108.1, GenBank Accession No: KC522107.1, GenBank Accession No: KC522106.1, GenBank Accession No: KC522105.1) Pipistrellus bat coronavirus HKU4 isolates (GenBank Accession No: KC522048.1, GenBank Accession No: KC522047.1, GenBank Accession No:KC522046.1, GenBank Accession No:KC522045.1, GenBank Accession No: KC522044.1, GenBank Accession No: KC522043.1, GenBank Accession No: KC522042.1, GenBank Accession No: KC522041.1, GenBank Accession No:KC522040.1 GenBank Accession No:KC522039.1, GenBank Accession No: KC522038.1, GenBank Accession No:KC522037.1, GenBank Accession No:KC522036.1, GenBank Accession No:KC522048.1 GenBank Accession No:KC522047.1 GenBank Accession No:KC522046.1 GenBank Accession No:KC522045.1 GenBank Accession No:KC522044.1 GenBank Accession No:KC522043.1 GenBank Accession No:KC522042.1 GenBank Accession No:KC522041.1 GenBank Accession No:KC522040.1, GenBank Accession No:KC522039.1 GenBank Accession No:KC522038.1 GenBank Accession No:KC522037.1 GenBank Accession No:KC522036.1, GenBank Accession No:KC522061.1 GenBank Accession No:KC522060.1 GenBank Accession No:KC522059.1 GenBank Accession No:KC522058.1 GenBank Accession No:KC522057.1 GenBank Accession No:KC522056.1 GenBank Accession No:KC522055.1 GenBank Accession No:KC522054.1 GenBank Accession No:KC522053.1 GenBank Accession No:KC522052.1 GenBank Accession No:KC522051.1 GenBank Accession No:KC522050.1 GenBank Accession No:KC522049.1 GenBank Accession No:KC522074.1, GenBank Accession No:KC522073.1 GenBank Accession No:KC522072.1 GenBank Accession No:KC522071.1 GenBank Accession No:KC522070.1 GenBank Accession No:KC522069.1 GenBank Accession No:KC522068.1 GenBank Accession No:KC522067.1, GenBank Accession No:KC522066.1 GenBank Accession No:KC522065.1 GenBank Accession No:KC522064.1, GenBank Accession No:KC522063.1, or GenBank Accession No:KC522062.1, as well as any other subgroup 2c coronavirus now known (e.g., as can be found in the GenBank® Database) or later identified, and any combination thereof.

Nonlimiting examples of a subgroup 1a coronavirus of this invention (e.g., said first coronavirus, second coronavirus and/or third coronavirus) include FCov.FIPV.79.1146.VR.2202 (GenBank Accession No. NV_007025), transmissible gastroenteritis virus (TGEV) (GenBank Accession No. NC_002306; GenBank Accession No. Q811789.2; GenBank Accession No. DQ811786.2; GenBank Accession No. DQ811788.1; GenBank Accession No. DQ811785.1; GenBank Accession No. X52157.1; GenBank Accession No. AJ011482.1; GenBank Accession No. KC962433.1; GenBank Accession No. AJ271965.2; GenBank Accession No. JQ693060.1; GenBank Accession No. KC609371.1; GenBank Accession No. JQ693060.1; GenBank Accession No. JQ693059.1; GenBank Accession No. JQ693058.1; GenBank Accession No. JQ693057.1; GenBank Accession No. JQ693052.1; GenBank Accession No. JQ693051.1; GenBank Accession No. JQ693050.1), porcine reproductive and respiratory syndrome virus (PRRSV) (GenBank Accession No. NC_001961.1; GenBank Accession No. DQ811787), as well as any other subgroup 1a coronavirus now known (e.g., as can be found in the GenBank® Database) or later identified, and any combination thereof.

Nonlimiting examples of a subgroup 1b coronavirus of this invention (e.g., said first coronavirus, second coronavirus and/or third coronavirus) include BtCoV.1A.AFCD62 (GenBank Accession No. NC_010437), BtCoV.1B.AFCD307 (GenBank Accession No. NC_010436), BtCov.HKU8.AFCD77 (GenBank Accession No. NC_010438), BtCoV.512.2005 (GenBank Accession No. DQ648858), porcine epidemic diarrhea virus PEDV.CV777 (GenBank Accession No. NC_003436, GenBank Accession No. DQ355224.1, GenBank Accession No. DQ355223.1, GenBank Accession No. DQ355221.1, GenBank Accession No. JN601062.1, GenBank Accession No. JN601061.1, GenBank Accession No. JN601060.1, GenBank Accession No.JN601059.1, GenBank Accession No. JN601058.1, GenBank Accession No.JN601057.1, GenBank Accession No.JN601056.1, GenBank Accession No.JN601055.1, GenBank Accession No. JN601054.1, GenBank Accession No.JN601053.1, GenBank Accession No. JN601052.1, GenBank Accession No. JN400902.1, GenBank Accession No.JN547395.1, GenBank Accession No. FJ687473.1, GenBank Accession No.FJ687472.1, GenBank Accession No. FJ687471.1, GenBank Accession No. FJ687470.1, GenBank Accession No. FJ687469.1, GenBank Accession No.FJ687468.1, GenBank Accession No. FJ687467.1, GenBank Accession No. FJ687466.1, GenBank Accession No. FJ687465.1, GenBank Accession No. FJ687464.1, GenBank Accession No. FJ687463.1, GenBank Accession No.FJ687462.1, GenBank Accession No. FJ687461.1, GenBank Accession No. FJ687460.1, GenBank Accession No. FJ687459.1, GenBank Accession No. FJ687458.1, GenBank Accession No. FJ687457.1, GenBank Accession No. FJ687456.1, GenBank Accession No. FJ687455.1, GenBank Accession No. FJ687454.1, GenBank Accession No. FJ687453 GenBank Accession No. FJ687452.1, GenBank Accession No. FJ687451.1, GenBank Accession No. FJ687450.1, GenBank Accession No.FJ687449.1, GenBank Accession No. AF500215.1, GenBank Accession No. KF476061.1, GenBank Accession No. KF476060.1, GenBank Accession No. KF476059.1, GenBank Accession No. KF476058.1, GenBank Accession No. KF476057.1, GenBank Accession No. KF476056.1, GenBank Accession No. KF476055.1, GenBank Accession No. KF476054.1, GenBank Accession No. KF476053.1, GenBank Accession No. KF476052.1, GenBank Accession No. KF476051.1, GenBank Accession No. KF476050.1, GenBank Accession No. KF476049.1, GenBank Accession No. KF476048.1, GenBank Accession No. KF177258.1, GenBank Accession No. KF177257.1, GenBank Accession No. KF177256.1, GenBank Accession No. KF177255.1), HCoV.229E (GenBank Accession No. NC_002645), HCoV.NL63.Amsterdam.I (GenBank Accession No. NC_005831), BtCoV.HKU2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), BtCoV.HKU2.GD.430.2006 (GenBank Accession No. EF203064), as well as any other subgroup 1b coronavirus now known (e.g., as can be found in the GenBank® Database) or later identified, and any combination thereof.

Nonlimiting examples of a subgroup 2a coronavirus of this invention (e.g., said first coronavirus, second coronavirus and/or third coronavirus) include HCoV.HKU1.C.N5 (GenBank Accession No. DQ339101), MHV.A59 (GenBank Accession No. NC_001846), PHEV.VW572 (GenBank Accession No. NC_007732), HCoV.OC43.ATCC.VR.759 (GenBank Accession No. NC_005147), bovine enteric coronavirus (BCoV.ENT) (GenBank Accession No. NC_003045), as well as any other subgroup 2a coronavirus now known (e.g., as can be found in the GenBank® Database) or later identified, and any combination thereof.

Nonlimiting examples of a subgroup 2d coronavirus of this invention (e.g., said first coronavirus, second coronavirus and/or third coronavirus) include BtCoV.HKU9.2 (GenBank Accession No. EF065514), BtCoV.HKU9.1 (GenBank Accession No. NC_009021), BtCoV.HkU9.3 (GenBank Accession No. EF065515), BtCoV.HKU9.4 (GenBank Accession No. EF065516), as well as any other subgroup 2d coronavirus now known (e.g., as can be found in the GenBank® Database) or later identified, and any combination thereof.

Nonlimiting examples of a subgroup 3 coronavirus of this invention (e.g., said first coronavirus, second coronavirus and/or third coronavirus) include Nonlimiting examples of a subgroup 3 coronavirus of this invention include IBV.Beaudette.IBV.p65 (GenBank Accession No. DQ001339), as well as any other subgroup 3 coronavirus now known (e.g., as can be found in the GenBank® Database) or later identified, and any combination thereof.

Representative nonlimiting examples of a chimeric coronavirus S protein of this invention are shown in Example 1, each of which provide an annotated amino acid sequence of a subgroup b coronavirus S protein with the regions annotated as described herein.

Thus, for example, in some embodiments of a chimeric coronavirus S protein of the present invention, the first coronavirus is subgroup 2b coronavirus SARS CoV2 (GenBank Accession No. MN908947), the second coronavirus is subgroup 2b coronavirus BtSARS.HKU3.1 (GenBank Accession No. DQ022305), and the third coronavirus is subgroup 2b coronavirus SARSCoV.Urbani (GenBank Accession No. AY278741).

In some embodiments, a chimeric coronavirus S protein of the present invention may comprise, consist essentially of, or consist of residues 16-1259 of the amino acid sequence SEQ ID NO:2, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

In some embodiments, a chimeric coronavirus S protein of the present invention may comprise, consist essentially of, or consist of the amino acid sequence SEQ ID NO:2, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 2. chimera #1-HKU3 NTD/SARS1 RBD/SARS2
S2 chimera
MFVFLVLLPLVSSQCGIISRKPQPKMAQVSSSRRGVYYNDDIFRSDVLH
LTQDYFLPFDSNLTQYFSLNVDSDRYTYFDNPILDFGDGVYFAATEKSN
VIRGWIFGSSFDNTTQSAVIVNNSTHIIIRVCNFNLCKEPMYTVSRGTQ
QNAWVYQSAFNCTYDRVEKSFQLDTTPKTGNFKDLREYVFKNRDGFLSV
YQTYTAVNLPRGLPTGFSVLKPILKLPFGINITSYRVVMAMFSQTTSNF
LPESAAYYVGNLKYSTFMLRFNENGTITDAVDCSQNPLAELKCTIKNFT
VEKGIYQTSNFRVQPTESIVRFPNITNLCPFGEVENATKFPSVYAWERK
KISNCVADYSVLYNSTFFSTFKCYGVSATKLNDLCFSNVYADSFVVKGD
DVRQIAPGQTGVIADYNYKLPDDFMGCVLAWNTRNIDATSTGNYNYKYR
YLRHGKLRPFERDISNVPFSPDGKPCTPPALNCYWPLNDYGFYTTTGIG
YQPYRVVVLSFELLNAPATVCGPKKSTNLVKNKCVNFNFNGLTGTGVLT
ESNKKFLPFQQFGRDIADTTDAVRDPQTLEILDITPCSFGGVSVITPGT
NTSNQVAVLYQDVNCTEVPVAIHADQLTPTWRVYSTGSNVFQTRAGCLI
GAEHVNNSYECDIPIGAGICASYQTQTNSPRRARSVASQSIIAYTMSLG
AENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFG
GFNFSQILPDPSKPSKRSFIEDLLFNKVTLADAGFIKQYGDCLGDIAAR
DLICAQKFNGLTVLPPLLTDEMIAQYTSALLAGTITSGWTFGAGAALQI
PFAMQMAYRFNGIGVTQNVLYENQKLIANQFNSAIGKIQDSLSSTASAL
GKLQDVVNQNAQALNTLVKQLSSNFGAISSVLNDILSRLDKVEAEVQID
RLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPL
QPELDSFKEELDKYFKNHTSPDVDLGDISGINASVVNIQKEIDRLNEVA
KNLNESLIDLQELGKYEQYIKWPWYIWLGFIAGLIAIVMVTIMLCCMTS
CCSCLKGCCSCGSCCKFDEDDSEPVLKGVKLHYT

Legend: HKU3, italics; SARS-CoV-1, bold; SARS-CoV-2, regular.

In some embodiments, a chimeric coronavirus S protein of the present invention may comprise, consist essentially of, or consist of the amino acid sequence SEQ ID NO:9, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 9. Chimera 1 HKU3-1 NTD/SARS-COV
RBD/SARS-COV-2 S2
MAISGVPVLGFFIIAVLMSAQESWAGIISRKPQPKMAQVSSSRRGVYYN
DDIFRSDVLHLTQDYFLPFDSNLTQYFSLNVDSDRYTYFDNPILDFGDG
VYFAATEKSNVIRGWIFGSSFDNTTQSAVIVNNSTHIIIRVCNFNLCKE
PMYTVSRGTQQNAWVYQSAFNCTYDRVEKSFQLDTTPKTGNFKDLREYV
FKNRDGFLSVYQTYTAVNLPRGLPTGFSVLKPILKLPFGINITSYRVVM
AMFSQTTSNFLPESAAYYVGNLKYSTFMLRFNENGTITDAVDCSQNPLA
ELKCTIKNFTVEKGIYQTSNFRVQPTESIVRFPNITNLCPFGEVENATK
FPSVYAWERKKISNCVADYSVLYNSTFFSTFKCYGVSATKLNDLCFSNV
YADSFVVKGDDVRQIAPGQTGVIADYNYKLPDDFMGCVLAWNTRNIDAT
STGNYNYKYRYLRHGKLRPFERDISNVPFSPDGKPCTPPALNCYWPLND
YGFYTTTGIGYQPYRVVVLSFELLNAPATVCGPKKSTNLVKNKCVNFNF
NGLTGTGVLTESNKKFLPFQQFGRDIADTTDAVRDPQTLEILDITPCSF
GGVSVITPGTNTSNQVAVLYQDVNCTEVPVAIHADQLTPTWRVYSTGSN
VFQTRAGCLIGAEHVNNSYECDIPIGAGICASYQTQTNSPRRARSVASQ
SIIAYTMSLGAENSVAYSNNSIAIPTNFTISVTTEILPVSMTKTSVDCT
MYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGW
TFGAGAALQIPFAMQMAYRFNGIGVTQNVLYENQKLIANQFNSAIGKIQ
DSLSSTASALGKLQDVVNQNAQALNTLVKQLSSNFGAISSVLNDILSRL
DKVEAEVQIDRLITGRLQSLQTYVTQQLIRAAEIRASANLAATKMSECV
LGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIG
IVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVM
VTIMLCCMTSCCSCLKGCCSCGSCCKFDEDDSEPVLKGVKLHYT

Legend: HKU3, italics; SARS-CoV-1, bold; SARS-CoV-2, regular.

It is to be understood that this example is not intended to be limiting and any of these subgroup 2b coronaviruses can be combined with any other subgroup 2b coronaviruses, or with any other coronaviruses, in any combination of first coronavirus, second coronavirus and third coronavirus.

As another example, in some embodiments of a chimeric coronavirus S protein of the present invention, the first coronavirus is subgroup 2b coronavirus SARSCoV.Urbani (GenBank Accession No. AY278741), the second coronavirus is subgroup 2b coronavirus SARSCoV.Urbani (GenBank Accession No. AY278741), and the third coronavirus is subgroup 2b coronavirus SARS CoV2 (GenBank Accession No. MN908947).

In some embodiments, a chimeric coronavirus S protein of the present invention may comprise, consist essentially of, or consist of residues 14-1256 of the amino acid sequence SEQ ID NO:3, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

In some embodiments, a chimeric coronavirus S protein of the present invention may comprise, consist essentially of, or consist of the amino acid sequence SEQ ID NO:3, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 3. chimera #2-SARS2 RBD/SARS1 S1 and
S2 chimera
MFIFLLFLTLTSGSDLDRCTTFDDVQAPNYTQHTSSMRGVYYPDEIFRS
DTLYLTQDLFLPFYSNVTGFHTINHTFGNPVIPFKDGIYFAATEKSNVV
RGWVFGSTMNNKSQSVIIINNSTNVVIRACNFELCDNPFFAVSKPMGTQ
THTMIFDNAFNCTFEYISDAFSLDVSEKSGNFKHLREFVFKNKDGFLYV
YKGYQPIDVVRDLPSGFNTLKPIFKLPLGINITNFRAILTAFSPAQDIW
GTSAAAYFVGYLKPTTFMLKYDENGTITDAVDCSQNPLAELKCSVKSFE
IDKGIYQTSNFRVVPSGDVVRFPNITNLCPFGEVENATRFASVYAWNRK
RISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYR
LFRKSNLKPFERDISTEIYQAGSTPCNGVEGENCYFPLQSYGFQPTNGV
GYQPYRVVVLSFELLHAPATVCGPKLSTDLIKNQCVNFNFNGLTGTGVL
TPSSKRFQPFQQFGRDVSDFTDSVRDPKTSEILDISPCSFGGVSVITPG
TNASSEVAVLYQDVNCTDVSTAIHADQLTPAWRIYSTGNNVFQTQAGCL
IGAEHVDTSYECDIPIGAGICASYHTVSLLRSTSQKSIVAYTMSLGADS
SIAYSNNTIAIPTNFSISITTEVMPVSMAKTSVDCNMYICGDSTECANL
LLQYGSFCTQLNRALSGIAAEQDRNTREVFAQVKQMYKTPTLKYFGGFN
FSQILPDPLKPTKRSFIEDLLFNKVTLADAGFMKQYGECLGDINARDLI
CAQKFNGLTVLPPLLTDDMIAAYTAALVSGTATAGWTFGAGAALQIPFA
MQMAYRFNGIGVTQNVLYENQKQIANQFNKAISQIQESLTTTSTALGKL
QDVVNQNAQALNTLVKQLSSNFGAISSVLNDILSRLDKVEAEVQIDRLI
TGRLQSLQTYVTQQLIRAAEIRASANLAATKMSECVLGQSKRVDFCGKG
YHLMSFPQAAPHGVVFLHVTYVPSQERNFTTAPAICHEGKAYFPREGVF
VFNGTSWFITQRNFFSPQIITTDNTFVSGNCDVVIGIINNTVYDPLQPE
LDSFKEELDKYFKNHTSPDVDLGDISGINASVVNIQKEIDRLNEVAKNL
NESLIDLQELGKYEQYIKWPWYVWLGFIAGLIAIVMVTILLCCMTSCCS
CLKGACSCGSCCKFDEDDSEPVLKGVKLHYT

Legend: SARS-CoV-2, bold; SARS-CoV-1, regular.

In some embodiments, a chimeric coronavirus S protein of the present invention may comprise, consist essentially of, or consist of the amino acid sequence SEQ ID NO:10, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 10. Chimera 2 SARS-COV-2 RBD/SARS-COV
NTD and S2
MAISGVPVLGFFIIAVLMSAQESWASDLDRCTTFDDVQAPNYTQHTSSM
RGVYYPDEIFRSDTLYLTQDLFLPFYSNVTGFHTINHTFGNPVIPFKDG
IYFAATEKSNVVRGWVFGSTMNNKSQSVIIINNSTNVVIRACNFELCDN
PFFAVSKPMGTQTHTMIFDNAFNCTFEYISDAFSLDVSEKSGNFKHLRE
FVFKNKDGFLYVYKGYQPIDVVRDLPSGFNTLKPIFKLPLGINITNFRA
ILTAFSPAQDIWGTSAAAYFVGYLKPTTFMLKYDENGTITDAVDCSQNP
LAELKCSVKSFEIDKGIYQTSNFRVVPSGDVVRFPNITNLCPFGEVFNA
TRFASVYAWNRKRISNCVADYSVLYNSASFSTFKCYGVSPTKLNDLCFT
NVYADSFVIRGDEVRQIAPGQTGKIADYNYKLPDDFTGCVIAWNSNNLD
SKVGGNYNYLYRLFRKSNLKPFERDISTEIYQAGSTPCNGVEGFNCYFP
LQSYGFQPTNGVGYQPYRVVVLSFELLHAPATVCGPKLSTDLIKNQCVN
FNFNGLTGTGVLTPSSKRFQPFQQFGRDVSDFTDSVRDPKTSEILDISP
CSFGGVSVITPGTNASSEVAVLYQDVNCTDVSTAIHADQLTPAWRIYST
GNNVFQTQAGCLIGAEHVDTSYECDIPIGAGICASYHTVSLLRSTSQKS
IVAYTMSLGADSSIAYSNNTIAIPTNFSISITTEVMPVSMAKTSVDCNM
YICGDSTECANLLLQYGSFCTQLNRALSGIAAEQDRNTREVFAQVKQMY
KTPTLKYFGGFNFSQILPDPLKPTKRSFIEDLLFNKVTLADAGFMKQYG
ECLGDINARDLICAQKFNGLTVLPPLLTDDMIAAYTAALVSGTATAGWT
FGAGAALQIPFAMQMAYRENGIGVTQNVLYENQKQIANQFNKAISQIQE
SLTTTSTALGKLQDVVNQNAQALNTLVKQLSSNFGAISSVLNDILSRLD
KVEAEVQIDRLITGRLQSLQTYVTQQLIRAAEIRASANLAATKMSECVL
GQSKRVDFCGKGYHLMSFPQAAPHGVVFLHVTYVPSQERNFTTAPAICH
EGKAYFPREGVFVFNGTSWFITQRNFFSPQIITTDNTFVSGNCDVVIGI
INNTVYDPLQPELDSFKEELDKYFKNHTSPDVDLGDISGINASVVNIQK
EIDRLNEVAKNLNESLIDLQELGKYEQYIKWPWYVWLGFIAGLIAIVMV
TILLCCMTSCCSCLKGACSCGSCCKFDEDDSEPVLKGVKLHYT

Legend: SARS-CoV-2, bold; SARS-CoV-1, regular.

It is to be understood that this example is not intended to be limiting and any of these subgroup 2b coronaviruses can be combined with any other subgroup 2b coronaviruses, or any other coronaviruses, in any combination of first coronavirus, second coronavirus and third coronavirus.

As another example, in some embodiments of a chimeric coronavirus S protein of the present invention, the first coronavirus is subgroup 2b coronavirus SARS CoV2 (GenBank Accession No. MN908947), the second coronavirus is subgroup 2b coronavirus SARS CoV2 (GenBank Accession No. MN908947), and the third coronavirus is subgroup 2b coronavirus SARSCoV.Urbani (GenBank Accession No. AY278741).

In some embodiments, a chimeric coronavirus S protein of the present invention may comprise, consist essentially of, or consist of residues 16-1272 of the amino acid sequence SEQ ID NO:4, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

In some embodiments, a chimeric coronavirus S protein of the present invention may comprise, consist essentially of, or consist of the amino acid sequence SEQ ID NO:4, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 4. chimera #3-SARS1 RBD/SARS2 S1 and
S2 chimera
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVEN
ATKFPSVYAWERKKISNCVADYSVLYNSTFFSTFKCYGVSATKLNDLCF
SNVYADSFVVKGDDVRQIAPGQTGVIADYNYKLPDDFMGCVLAWNTRNI
DATSTGNYNYKYRYLRHGKLRPFERDISNVPFSPDGKPCTPPALNCYWP
LNDYGFYTTTGIGYQPYRVVVLSFELLNAPATVCGPKKSTNLVKNKCVN
FNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYST
GSNVFQTRAGCLIGAEHVNNSYECDIPIGAGICASYQTQTNSPRRARSV
ASQSIIAYTMSLGAENSVAYSNNSIAIPTNFTISVTTEILPVSMTKTSV
DCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFI
KQYGDCLGDIAARDLICAQKENGLTVLPPLLTDEMIAQYTSALLAGTIT
SGWTFGAGAALQIPFAMQMAYRFNGIGVTQNVLYENQKLIANQFNSAIG
KIQDSLSSTASALGKLQDVVNQNAQALNTLVKQLSSNFGAISSVLNDIL
SRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDV
VIGIVNNTVYDPLQPELDSFKEELDKYFKNHTSPDVDLGDISGINASVV
NIQKEIDRLNEVAKNLNESLIDLQELGKYEQYIKWPWYIWLGFIAGLIA
IVMVTIMLCCMTSCCSCLKGCCSCGSCCKFDEDDSEPVLKGVKLHYT

Legend: SARS-CoV-1, bold; SARS-CoV-2, regular.

In some embodiments, a chimeric coronavirus S protein of the present invention may comprise, consist essentially of, or consist of the amino acid sequence SEQ ID NO.4, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 11. Chimera 3 SARS-COV RBD/SARS-COV-2
NTD and S2
MAISGVPVLGFFIIAVLMSAQESWAVNLTTRTQLPPAYTNSFTRGVYYP
DKVFRSSVLHSTQDLFLPFFSNVTWFHAIHVSGTNGTKRFDNPVLPFND
GVYFASTEKSNIIRGWIFGTTLDSKTQSLLIVNNATNVVIKVCEFQFCN
DPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINI
TRFQTLLALHRSYLTPGDSSSGWTAGAAAYYVGYLQPRTFLLKYNENGT
ITDAVDCALDPLSETKCTLKSFTVEKGIYQTSNFRVQPTESIVRFPNIT
NLCPFGEVENATKFPSVYAWERKKISNCVADYSVLYNSTFFSTFKCYGV
SATKLNDLCFSNVYADSFVVKGDDVRQIAPGQTGVIADYNYKLPDDFMG
CVLAWNTRNIDATSTGNYNYKYRYLRHGKLRPFERDISNVPFSPDGKPC
TPPALNCYWPLNDYGFYTTTGIGYQPYRVVVLSFELLNAPATVCGPKKS
TNLVKNKCVNFNFNGLTGTGVLTESNKKFLPFQQFGRDIADTTDAVRDP
QTLEILDITPCSFGGVSVITPGTNTSNQVAVLYQDVNCTEVPVAIHADQ
LTPTWRVYSTGSNVFQTRAGCLIGAEHVNNSYECDIPIGAGICASYQTQ
TNSPRRARSVASQSIIAYTMSLGAENSVAYSNNSIAIPTNFTISVTTEI
LPVSMTKTSVDCTMYICGDSTECSNLLLQYGSFCTQLNRALTGIAVEQD
KNTQEVFAQVKQIYKTPPIKDFGGFNFSQILPDPSKPSKRSFIEDLLFN
KVTLADAGFIKQYGDCLGDIAARDLICAQKFNGLTVLPPLLTDEMIAQY
TSALLAGTITSGWTFGAGAALQIPFAMQMAYRFNGIGVTQNVLYENQKL
IANQFNSAIGKIQDSLSSTASALGKLQDVVNQNAQALNTLVKQLSSNFG
AISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVP
AQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLG
DISGINASVVNIQKEIDRLNEVAKNLNESLIDLQELGKYEQYIKWPWYI
WLGFIAGLIAIVMVTIMLCCMTSCCSCLKGCCSCGSCCKFDEDDSEPVL
KGVKLHYT

Legend: SARS-CoV-1, bold; SARS-CoV-2, regular.

It is to be understood that this example is not intended to be limiting and any of these subgroup 2b coronaviruses can be combined with any other subgroup 2b coronaviruses, or any other coronaviruses, in any combination of first coronavirus, second coronavirus and third coronavirus.

As another example, in some embodiments of a chimeric coronavirus S protein of the present invention, the first coronavirus is subgroup 2b coronavirus SARS CoV2 (GenBank Accession No. MN908947), the second coronavirus is subgroup 2b coronavirus SARS CoV2 (GenBank Accession No. MN908947), and the third coronavirus is subgroup 2b coronavirus Rs SHC014 (GenBank® Accession No. KC881005).

In some embodiments, a chimeric coronavirus S protein of the present invention may comprise, consist essentially of, or consist of residues 16-1272 of the amino acid sequence SEQ ID NO:5, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

In some embodiments, a chimeric coronavirus S protein of the present invention may comprise, consist essentially of, or consist of the amino acid sequence SEQ ID NO:5, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 5. chimera #4-SCH014 RBD/SARS2 S1 and
S2 chimera
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVEN
ATTFPSVYAWERKRISNCVADYSVLYNSTSFSTFKCYGVSATKLNDLCF
SNVYADSFVVKGDDVRQIAPGQTGVIADYNYKLPDDFLGCVLAWNTNSK
DSSTSGNYNYLYRWVRRSKLNPYERDLSNDIYSPGGQSCSAVGPNCYNP
LRPYGFFTTAGVGHQPYRVVVLSFELLNAPATVCGPKKSTNLVKNKCVN
FNFNGLTGTGVLTESNKKFLPFQQFGRDIADTTDAVRDPQTLEILDITP
CSFGGVSVITPGTNTSNQVAVLYQDVNCTEVPVAIHADQLTPTWRVYST
GSNVFQTRAGCLIGAEHVNNSYECDIPIGAGICASYQTQTNSPRRARSV
ASQSIIAYTMSLGAENSVAYSNNSIAIPTNFTISVTTEILPVSMTKTSV
DCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFI
KQYGDCLGDIAARDLICAQKENGLTVLPPLLTDEMIAQYTSALLAGTIT
SGWTFGAGAALQIPFAMQMAYRENGIGVTQNVLYENQKLIANQFNSAIG
KIQDSLSSTASALGKLQDVVNQNAQALNTLVKQLSSNFGAISSVLNDIL
SRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDV
VIGIVNNTVYDPLQPELDSFKEELDKYFKNHTSPDVDLGDISGINASVV
NIQKEIDRLNEVAKNLNESLIDLQELGKYEQYIKWPWYIWLGFIAGLIA
IVMVTIMLCCMTSCCSCLKGCCSCGSCCKFDEDDSEPVLKGVKLHYT

Legend: SCH014, bold; SARS-CoV-2, regular.

In some embodiments, a chimeric coronavirus S protein of the present invention may comprise, consist essentially of, or consist of the amino acid sequence SEQ ID NO:12, or a sequence at least about 70% identical thereto (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical thereto).

SEQ ID NO: 12. Chimera 4 RsSHC014 RBD/Remaining
Spike SARS-COV-2
MAISGVPVLGFFIIAVLMSAQESWAVNLTTRTQLPPAYTNSFTRGVYYP
DKVFRSSVLHSTQDLFLPFFSNVTWFHAIHVSGTNGTKRFDNPVLPFND
GVYFASTEKSNIIRGWIFGTTLDSKTQSLLIVNNATNVVIKVCEFQFCN
DPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINI
TRFQTLLALHRSYLTPGDSSSGWTAGAAAYYVGYLQPRTFLLKYNENGT
ITDAVDCALDPLSETKCTLKSFTVEKGIYQTSNFRVQPTESIVRFPNIT
NLCPFGEVENATTFPSVYAWERKRISNCVADYSVLYNSTSFSTFKCYGV
SATKLNDLCFSNVYADSFVVKGDDVRQIAPGQTGVIADYNYKLPDDFLG
CVLAWNTNSKDSSTSGNYNYLYRWVRRSKLNPYERDLSNDIYSPGGQSC
SAVGPNCYNPLRPYGFFTTAGVGHQPYRVVVLSFELLNAPATVCGPKKS
TNLVKNKCVNFNFNGLTGTGVLTESNKKFLPFQQFGRDIADTTDAVRDP
QTLEILDITPCSFGGVSVITPGTNTSNQVAVLYQDVNCTEVPVAIHADQ
LTPTWRVYSTGSNVFQTRAGCLIGAEHVNNSYECDIPIGAGICASYQTQ
TNSPRRARSVASQSIIAYTMSLGAENSVAYSNNSIAIPTNFTISVTTEI
LPVSMTKTSVDCTMYICGDSTECSNLLLQYGSFCTQLNRALTGIAVEQD
KNTQEVFAQVKQIYKTPPIKDFGGFNFSQILPDPSKPSKRSFIEDLLFN
KVTLADAGFIKQYGDCLGDIAARDLICAQKFNGLTVLPPLLTDEMIAQY
TSALLAGTITSGWTFGAGAALQIPFAMQMAYRFNGIGVTQNVLYENQKL
IANQFNSAIGKIQDSLSSTASALGKLQDVVNQNAQALNTLVKQLSSNFG
AISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVP
AQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLG
DISGINASVVNIQKEIDRLNEVAKNLNESLIDLQELGKYEQYIKWPWYI
WLGFIAGLIAIVMVTIMLCCMTSCCSCLKGCCSCGSCCKFDEDDSEPVL
KGVKLHYT

Legend: SCH014, bold; SARS-CoV-2, regular.

It is to be understood that this example is not intended to be limiting and any of these subgroup 2b coronaviruses can be combined with any other subgroup 2b coronaviruses, or any other coronaviruses, in any combination of first coronavirus, second coronavirus and third coronavirus.

Although the examples set forth above describe chimeric S proteins produced from subgroup 2b coronaviruses, it is to be understood that a chimeric coronavirus S protein of this invention can be made from any combination of at least two (e.g., two or three) different coronaviruses from any subgroup, including subgroup 1a, subgroup 1b, subgroup 2a, subgroup 2d and subgroup 3, in addition to subgroup 2b and subgroup 2c. The same arrangement of the backbone, first region and/or second region as described above would be applicable to a chimeric coronavirus S protein of any subgroup.

Furthermore, the chimeric coronavirus S proteins produced from the respective coronavirus subgroups 1a, 1b, 2a, 2b, 2c, 2d and 3 can be included in the methods and compositions of this invention in any combination and/or in any ratio relative to one another, as would be well understood to one of ordinary skill in the art.

The amino acid residue positions of the substitutions that can be made to produce the desired chimeric S protein can be readily determined by one of ordinary skill in the art according to the teachings herein and according to protocols well known in the art. The amino acid residue numbering provided in the amino acid sequences set forth here is based on the reference sequence of SARS-CoV-2 wild type S protein, as provided herein (SEQ ID NO:1). However it would be readily understood by one of ordinary skill in the art that the equivalent amino acid positions in other coronavirus S protein sequences can be readily identified and employed in the production of the chimeric S proteins of this invention.

It would be understood that the modifications described above provide multiple examples of how the amino acid sequences described herein can be obtained and that, due to the degeneracy of the amino acid codons, numerous other modifications can be made to a nucleotide sequence encoding an S protein or fragment thereof to obtain the desired amino acid sequence. The present invention provides additional non limiting examples of nucleic acids and/or polypeptides of this invention that can be used in the compositions and methods described herein in the SEQUENCES section provided herein.

The present invention further provides an isolated nucleic acid molecule encoding the chimeric coronavirus S protein of this invention. In some embodiments, a nucleic acid molecule of this invention may be a cDNA molecule. In some embodiments, a nucleic acid molecule of this invention may be an mRNA molecule.

Also provided is a vector, plasmid or other nucleic acid construct comprising the isolated nucleic acid molecule of this invention.

A vector can be any suitable means for delivering a polynucleotide to a cell. A vector of this invention can be an expression vector that contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, poxvirus, vaccinia virus, adenovirus, retrovirus, alphavirus and/or adeno-associated virus nucleic acid. The nucleic acid molecule or vector of this invention can also be in a liposome or a delivery vehicle, which can be taken up by a cell via receptor-mediated or other type of endocytosis. The nucleic acid molecule of this invention can be in a cell, which can be a cell expressing the nucleic acid whereby a chimeric S protein of this invention is produced in the cell (e.g., a host cell). In addition, the vector of this invention can be in a cell, which can be a cell expressing the nucleic acid of the vector whereby a chimeric S protein of this invention is produced in the cell. It is also contemplated that the nucleic acid molecules and/or vectors of this invention can be present in a host organism (e.g., a transgenic organism), which expresses the nucleic acids of this invention and produces the chimeric S protein of this invention. In some embodiments, the vector is a plasmid, a viral vector, a bacterial vector, an expression cassette, a transformed cell, or a nanoparticle. For example, in some embodiments, a chimeric coronavirus S protein of the present invention may be used in combination (e.g., in scaffold(s) and/or conjugated with) other molecules such as, but not limited to, nanoparticles, e.g., as delivery devices.

Types of nanoparticles of this invention for use as a vector and/or delivery device include, but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales. Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art. These particles may be generated and/or have materials be absorbed, encapsulated, or chemically bound through known mechanisms in the art.

In some embodiments, a nanoparticle vector of the present invention may be an mRNA lipid nanoparticle (mRNA-LNP), a nucleic acid vaccine (NAV), or other nucleic acid lipid nanoparticle compositions, such as described in U.S. Pat. Nos. 9,868,692; 9,950,065; 10,041,091; 10,576,146; 10,702,600; WO2015/164674; US2019/0351048; US2020/297634; WO2020/097548; and Buschmann et al. 2021 Vaccines 9(65) doi.org/10.3390/vaccines9010065; Laczkó et al. 2020 Immunity 53:724-732; and Pardi et al. 2018 Nat. Rev. Drug Discov. 17:261-279, the disclosures of each of which are incorporated herein by reference in their entireties.

In some embodiments, a nanoparticle vector of the present invention may comprise an isolated nucleic acid molecule encoding one or more of the chimeric coronavirus S proteins of the present invention. In some embodiments, a nanoparticle vector of the present invention may be a “multiplexed” vector, e.g., may comprise one or more isolated nucleic acid molecules, each isolated nucleic acid molecule encoding a different one or more of the chimeric coronavirus S proteins of the present invention, e.g., comprising at least two, at least three, at least four, at least five, at least six, at least seven, at least eight, at least nine, at least ten, 11, 12, 13, 14, 15, 16, 17, 18, 19, or 20 or more isolated nucleic acid molecules or any value or range therein, each isolated nucleic acid molecule encoding a different chimeric S protein of the present invention. For example, in some embodiments, a multiplexed vector of the present invention may comprise at least one chimeric S protein, at least three chimeric S proteins, at least 10 chimeric S proteins, at least 15 chimeric S proteins, or at least 20 chimeric S proteins of the present invention, or at least one to three chimeric S proteins, at least one to 10 chimeric S proteins, at least three to 20 chimeric S proteins, or at least one to 15 chimeric S proteins of the present invention.

Compositions comprising two or more chimeric coronavirus S proteins of the present invention and/or isolated nucleic acid molecules encoding the same may comprise the two or more chimeric coronavirus S proteins at a ratio of about 1:1, about 2:1, about 3:1, about 4:1, about 5:1, about 6:1, about 7:1, about 8:1, about 9:1, or about 10:1 or any value or range or range therein, e.g., about 1:1 ratio, e.g., about 1:1:1, about 1:1:1:1, about 1:1:1:1:1, about 1:1:1:1:1:1, about 1:1:1:1:1:1:1, about 1:1:1:1:1:1:1:1, about 1:1:1:1:1:1:1:1:1, about 1:1:1:1:1:1:1:1:1:1, about 1:1:1:1:1:1:1:1:1:1:1, about 1:1:1:1:1:1:1:1:1:1:1:1, about 1:1:1:1:1:1:1:1:1:1:1:1:1, 1:1:1:1:1:1:1:1:1:1:1, about 1:1:1:1:1:1:1:1:1:1:1:1, about 1:1:1:1:1:1:1:1:1:1:1:1:1:1:1:1, about 1:1:1:1:1:1:1:1:1:1:1:1:1:1:1::1:1, about 1:1:1:1:1:1:1:1:1:1:1:1:1:1:1:1:1:1, about 1:1:1:1:1:1:1:1:1:1:1:1:1:1:1:1:1:1:1, about 1:1:1:1:1:1:1:1:11:1:1:1:1:1:1:1::1:1, or about 2:1:1, about 1:2:1, about 1:1:2, about 1:1:10, about 1:10:1, or about 10:1:1, etc., or any value or range therein.

Further provided herein is a Venezuelan equine encephalitis (VEE) replicon particle (VRP) comprising an isolated nucleic acid molecule encoding the chimeric coronavirus S protein of this invention.

In addition, the present invention provides a virus like particle (VLP) comprising the chimeric coronavirus S protein of any of this invention and a matrix protein of any virus that can form a VLP.

The present invention also provides a coronavirus particle comprising the chimeric coronavirus S protein of this invention.

Also provided is a cell (e.g., an isolated cell) comprising the vectors, nucleic acid molecules, VLPs, VRPs, and/or coronavirus particles of the invention.

Additionally provided herein is a population of any of the VLPs, VRPs and/or coronavirus particles of this invention, as well as a population of virus particles that are used as viral vectors encoding the chimeric coronavirus spike protein of this invention.

The chimeric coronavirus S proteins of this invention can be produced as recombinant proteins, e.g., in a eukaryotic cell system for recombination protein production.

The invention also provides immunogenic compositions comprising the cells, vectors, nucleic acid molecules, VLPs, VRPs, coronavirus particles and/or populations of the invention. The composition can further comprise a pharmaceutically acceptable carrier.

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

In some embodiments, the present invention provides a pharmaceutical composition comprising a chimeric coronavirus S protein, nucleic acid molecule (e.g., an mRNA molecule), vector, VRP, VLP, coronavirus particle, population and/or composition of the present invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Immunogenic compositions comprising a chimeric coronavirus S protein, nucleic acid molecule, vector, VRP, VLP, coronavirus particle, population and/or composition of the present invention may be formulated by any means known in the art. Such compositions, especially vaccines, are typically prepared as injectables, either as liquid solutions or suspensions. Solid forms suitable for solution in, or suspension in, liquid prior to injection may also be prepared. Lyophilized preparations are also suitable. In some embodiments, a pharmaceutical composition of the present invention may be a vaccine formulation, e.g., may comprise chimeric coronavirus S protein, nucleic acid molecule, vector, VRP, VLP, coronavirus particle, population and/or composition of the present invention and adjuvant(s), optionally in a vaccine diluent. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

In some embodiments, a pharmaceutical composition comprising a chimeric coronavirus S protein, nucleic acid molecule, vector, VRP, VLP, coronavirus particle, population and/or composition of the present invention may further comprise additional agents, such as, but not limited to, additional antigen as part of a cocktail in a vaccine, e.g., a multi-component vaccine wherein the vaccine may additionally include peptides, cells, virus, viral peptides, inactivated virus, etc. Thus, in some embodiments, a pharmaceutical composition comprising chimeric coronavirus S protein, nucleic acid molecule, vector, VRP, VLP, coronavirus particle, population and/or composition of the present invention, a pharmaceutically acceptable carrier may further comprise additional viral antigen, e.g., SARS-CoV-2 antigen in the form of peptides, peptoids, whole SARS-CoV-2 virus (e.g., live attenuated and/or inactivated virus), and/or SARS-CoV-2 virus-comprising cells (e.g., cells modified to express SARS-CoV-2 viral components, e.g., SARS-CoV-2 viral peptides).

In some embodiments, a pharmaceutical composition comprising a chimeric coronavirus S protein, nucleic acid molecule, vector, VRP, VLP, coronavirus particle, population and/or composition of the present invention, and a pharmaceutically acceptable carrier may further comprise an adjuvant. As used herein, “suitable adjuvant” describes an adjuvant capable of being combined with a chimeric coronavirus S protein, nucleic acid molecule, vector, VRP, VLP, coronavirus particle, population and/or composition of this invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

### Methods

The nucleic acids, proteins, peptides, viruses, vectors, particles, antibodies, VLPs, VRPs, populations, and/or compositions of this invention are intended for use as therapeutic agents and immunological reagents, for example, as antigens, immunogens, vaccines, and/or nucleic acid delivery vehicles. The compositions described herein can be formulated for use as reagents (e.g., to produce antibodies) and/or for administration in a pharmaceutical carrier in accordance with known techniques. See, e.g., Remington, The Science and Practice of Pharmacy (latest edition).

In embodiments of this invention wherein a chimeric coronavirus S protein is being administered, delivered and/or introduced into a subject, e.g., to elicit or induce an immune response, the protein can be administered, delivered and/or introduced into the subject as a protein present in an inactivated (e.g., inactivated through UV irradiation or formalin treatment) coronavirus. The protein or active fragment thereof of this invention can be administered, delivered and/or introduced into the subject according to any method now known or later identified for administration, introduction and/or delivery of protein or active fragment thereof, as would be well known to one of ordinary skill in the art. Nonlimiting examples include administration of the protein or fragment with a protease inhibitor or other agent to protect it from degradation and/or with a polyalkylene glycol moiety (e.g., polyethylene glycol).

In one aspect, the present invention provides a method of producing an immune response to a coronavirus in a subject, comprising administering to the subject an effective amount of a chimeric coronavirus S protein, nucleic acid molecule (e.g., mRNA molecule), vector, VRP, VLP, coronavirus particle, population and/or composition of the present invention, in any combination, thereby producing an immune response to a coronavirus in the subject.

In another aspect, the present invention provides a method of treating a coronavirus infection in a subject in need thereof, comprising administering to the subject an effective amount of a chimeric coronavirus S protein, nucleic acid molecule (e.g., mRNA molecule), vector, VRP, VLP, coronavirus particle, population and/or composition of the present invention, in any combination, in any combination, thereby treating a coronavirus infection in the subject.

In another aspect, the present invention provides a method of preventing a disease or disorder caused by a coronavirus infection in a subject, comprising administering to the subject an effective amount of a chimeric coronavirus S protein, nucleic acid molecule (e.g., mRNA molecule), vector, VRP, VLP, coronavirus particle, population and/or composition of the present invention, in any combination, thereby preventing a disease or disorder caused by a coronavirus infection in the subject.

In another aspect, the present invention provides a method of protecting a subject from the effects of coronavirus infection, comprising administering to the subject an effective amount of a chimeric coronavirus S protein, nucleic acid molecule (e.g., mRNA molecule), vector, VRP, VLP, coronavirus particle, population and/or composition of the present invention, in any combination, thereby protecting the subject from the effects of coronavirus infection.

The chimeric coronavirus S proteins of this invention can be used to immunize a subject against infection by a newly emerging coronavirus, as well as treat a subject infected with a newly emerging coronavirus.

Further provided herein is a method of identifying a coronavirus S protein for administration to elicit an immune response to coronavirus in a subject (e.g., a subject infected by a coronavirus and/or a subject at risk of coronavirus infection and/or to a subject for whom eliciting an immune response to a coronavirus is needed or desired), comprising: a) contacting a sample obtained from a subject known to be or suspected of being infected with a coronavirus with a chimeric coronavirus S protein of the present invention under conditions whereby an antigen/antibody complex can form; and b) detecting formation of an antigen/antibody complex, whereby detection of formation of the antigen/antibody complex comprising the chimeric coronavirus S protein identifies the presence in said sample of antibodies that bind an S protein of at least one of the coronaviruses of said chimeric coronavirus S protein (e.g., said first, second, or third coronavirus), thereby identifying a coronavirus S protein for administration to a subject for whom eliciting an immune response to a coronavirus is needed or desired. In some embodiments, the method may further comprise the step of administering the identified coronavirus S protein to a subject (e.g., administering the coronavirus S protein identified according to the method to the subject of (a) and/or to a subject at risk of coronavirus infection and/or to a subject infected with a coronavirus and/or to a subject for whom eliciting an immune response to a coronavirus is needed or desired).

Further provided herein is a method of detecting an antibody that binds a coronavirus S protein in a sample, comprising: a) contacting the sample with the coronavirus S protein under conditions whereby an antigen/antibody complex can form; and b) detecting the formation of an antigen/antibody complex, thereby detecting the presence in the sample of an antibody that binds a coronavirus S protein. In some embodiments, the sample is from a subject. In some embodiments, the subject is known to have been or is suspected of having been infected by a coronavirus.

The chimeric coronavirus S protein of the present invention may be administered in any frequency, amount, and/or route as needed to elicit an effective prophylactic and/or therapeutic effect in a subject (e.g., in a subject in need thereof) as described herein. In certain embodiments, the chimeric coronavirus S protein, nucleic acid molecule, vector, VRP, VLP, coronavirus particle, population and/or composition is administered/delivered to the subject, e.g., systemically (e.g., intravenously). In particular embodiments, more than one administration (e.g., two, three, four or more administrations) may be employed to achieve the desired level of protein expression over a period of various intervals, e.g., daily, weekly, monthly, yearly, etc. The most suitable route in any given case will depend on the nature and severity of the condition being treated and on the nature of the particular delivery method that is being used. In embodiments wherein a vector is used, the vector will typically be administered in a liquid formulation by direct injection (e.g., stereotactic injection) to the desired region or tissues. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. For example, controlled release of parvovirus and AAV vectors is described in international patent publication WO 01/91803, which is incorporated by reference herein for these teachings.

Administration may be by any suitable means, such as intraperitoneally, intramuscularly, intranasally, intravenously, intradermally (e.g., by a gene gun), intrarectally and/or subcutaneously. The compositions herein may be administered via a skin scarification method, and/or transdermally via a patch or liquid. The compositions can be delivered subdermally in the form of a biodegradable material that releases the compositions over a period of time. As further non-limiting examples, the route of administration can be by inhalation (e.g., oral and/or nasal inhalation), oral, buccal (e.g., sublingual), rectal, vaginal, topical (including administration to the airways), intraocular, by parenteral (e.g., intramuscular [e.g., administration to skeletal muscle], intravenous, intra-arterial, intraperitoneal and the like), subcutaneous (including administration into the footpad), intrapleural, intracerebral, intrathecal, intraventricular, intra-aural, intra-ocular (e.g., intra-vitreous, sub-retinal, anterior chamber) and peri-ocular (e.g., sub-Tenon's region) routes or any combination thereof.

In some embodiments, the chimeric coronavirus S protein can be administered to a subject as a nucleic acid molecule, which can be a naked nucleic acid molecule or a nucleic acid molecule present in a vector (e.g., a delivery vector, which in some embodiments can be a viral vector, such as a VRP). The nucleic acids and vectors of this invention can be administered orally, intranasally, parenterally (e.g., intravenously), by intramuscular injection, by intraperitoneal injection, transdermally, extracorporeally, topically or the like. In the methods described herein which include the administration and uptake of exogenous DNA into the cells of a subject (i.e., gene transduction or transfection), the nucleic acids of the present invention can be in the form of naked DNA or the nucleic acids can be in a vector for delivering the nucleic acids to the cells for expression of the polypeptides and/or fragments of this invention. The vector can be a commercially available preparation or can be constructed in the laboratory according to methods well known in the art.

Delivery of the nucleic acid or vector to cells can be via a variety of mechanisms, including but not limited to recombinant vectors including bacterial, viral, and fungal vectors, liposomal delivery agents, nanoparticles, and gene gun related mechanisms.

In some embodiments, the nucleic acid molecules encoding the chimeric coronavirus S proteins of this invention can be part of a recombinant nucleic acid construct comprising any combination of restriction sites and/or functional elements as are well known in the art that facilitate molecular cloning and other recombinant nucleic acid manipulations. Thus, the present invention further provides a recombinant nucleic acid construct comprising a nucleic acid molecule encoding a chimeric coronavirus S protein of this invention. The nucleic acid molecule encoding the chimeric coronavirus S protein of this invention can be any nucleic acid molecule that functionally encodes the chimeric coronavirus S protein of this invention. To functionally encode the chimeric coronavirus S protein (i.e., allow the nucleic acids to be expressed), the nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Non-limiting examples of expression control sequences that can be present in a nucleic acid molecule of this invention include promoters derived from metallothionine genes, actin genes, immunoglobulin genes, CMV, SV40, adenovirus, bovine papilloma virus, etc. A nucleic acid molecule encoding a selected chimeric coronavirus S protein can readily be determined based upon the genetic code for the amino acid sequence of the selected polypeptide and/or fragment of interest included in the chimeric coronavirus S protein, and many nucleic acids will encode any selected polypeptide and/or fragment. Modifications in the nucleic acid sequence encoding the polypeptide and/or fragment are also contemplated. Modifications that can be useful are modifications to the sequences controlling expression of the polypeptide and/or fragment to make production of the polypeptide and/or fragment inducible or repressible as controlled by the appropriate inducer or repressor. Such methods are standard in the art. The nucleic acid molecule and/or vector of this invention can be generated by means standard in the art, such as by recombinant nucleic acid techniques and/or by synthetic nucleic acid synthesis or in vitro enzymatic synthesis.

The nucleic acids and/or vectors of this invention can be transferred into a host cell (e.g., a prokaryotic or eukaryotic cell) by well-known methods, which vary depending on the type of cell host. For example, calcium chloride transfection is commonly used for prokaryotic cells, whereas calcium phosphate treatment, transduction, cationic lipid treatment and/or electroporation can be used for other cell hosts.

As another example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega, Madison, WI), as well as other liposomes developed according to procedures standard in the art. In addition, the nucleic acid or vector of this invention can be delivered in vivo by electroporation, the technology for which is available from Genetronics, Inc. (San Diego, CA) as well as by means of a SONOPORATION machine (ImaRx Pharmaceutical Corp., Tucson, AZ).

As another example, vector delivery can be via a viral system, such as a retroviral vector system, which can package a recombinant retroviral genome. The recombinant retrovirus can then be used to infect and thereby deliver to the infected cells nucleic acid encoding the polypeptide and/or fragment of this invention. The exact method of introducing the exogenous nucleic acid into mammalian cells is, of course, not limited to the use of retroviral vectors. Other techniques are widely available for this procedure including the use of adenoviral vectors, alphaviral vectors (e.g., VRPs), adeno-associated viral (AAV) vectors, lentiviral vectors, pseudotyped retroviral vectors and vaccinia viral vectors, as well as any other viral vectors now known or developed in the future. Physical transduction techniques can also be used, such as liposome delivery and receptor-mediated and other endocytosis mechanisms. This invention can be used in conjunction with any of these or other commonly used gene transfer methods.

If ex vivo methods are employed, cells or tissues can be removed and maintained outside the body according to standard protocols well known in the art. The nucleic acids and vectors of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

Parenteral administration of the peptides, polypeptides, nucleic acids and/or vectors of the present invention, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained. See, e.g., U.S. Pat. No. 3,610,795, which is incorporated by reference herein in its entirety.

In some embodiments, the compositions of the invention can be administered with and/or further comprise one or more than one adjuvant. The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

An adjuvant for use with the present invention, such as, for example, an immunostimulatory cytokine, can be administered before, concurrent with, and/or within a few hours, several hours, and/or 1, 2, 3, 4, 5, 6, 7, 8, 9, and/or 10 days before and/or after the administration of a composition of the invention to a subject.

Furthermore, any combination of adjuvants, such as immunostimulatory cytokines, can be co-administered to the subject before, after and/or concurrent with the administration of an immunogenic composition of the invention. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

In some embodiments, the methods of the present invention may further comprise administering a chimeric coronavirus S protein, nucleic acid molecule, vector, VRP, VLP, coronavirus particle, population and/or composition of the present invention, a pharmaceutically acceptable carrier, and, optionally, other medicinal agents, therapeutic agents, pharmaceutical agents, stabilizing agents, buffers, carriers, adjuvants, diluents, etc. In some embodiments, the methods of the present invention may further comprise administering additional agent(s) such as, but not limited to, additional antigen as part of a cocktail in a vaccine, e.g., a multi-component cocktail vaccine wherein the vaccine may additionally include peptides, cells, virus, viral peptides, inactivated virus, etc. Thus, in some embodiments, the methods of the present invention may further comprise administering additional viral antigen, e.g., coronavirus antigen in the form of peptides, peptoids, whole virus (e.g., live attenuated and/or inactivated virus), and/or virus-comprising cells (e.g., cells modified to express viral components, e.g., viral peptides).

Injectables can be prepared in conventional forms, either as liquid solutions or suspensions, solid forms suitable for solution or suspension in liquid prior to injection, or as emulsions. Alternatively, one may administer the vector in a local rather than systemic manner, for example, in a depot or sustained-release formulation. Further, the virus vector can be delivered dried to a surgically implantable matrix such as a bone graft substitute, a suture, a stent, and the like (e.g., as described in U.S. Pat. No. 7,201,898).

Pharmaceutical compositions suitable for oral administration can be presented in discrete units, such as capsules, cachets, lozenges, or tablets, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a vector of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions suitable for parenteral administration can comprise sterile aqueous and non-aqueous injection solutions of the composition of this invention, which preparations are optionally isotonic with the blood of the intended recipient. These preparations can contain antioxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 pg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration. The compositions herein may also be administered via a skin scarification method, or transdermally via a patch or liquid. The compositions may be delivered subdermally in the form of a biodegradable material that releases the compositions over a period of time. The most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for rectal administration can be presented as unit dose suppositories. These can be prepared by admixing the composition with one or more conventional solid carriers, such as for example, cocoa butter, and then shaping the resulting mixture.

Pharmaceutical compositions of this invention suitable for topical application to the skin can take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharm. Res. 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

The delivery methods disclosed herein may be administered to the lungs of a subject by any suitable means, for example, by administering an aerosol suspension of respirable particles comprised of the vectors, which the subject inhales. The respirable particles may be liquid or solid. Aerosols of liquid particles comprising the virus vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the vectors may likewise be produced with any solid particulate medicament aerosol generator, by techniques known in the pharmaceutical art.

The compositions of this invention can be optimized and combined with other vaccination regimens to provide the broadest (i.e., covering all aspects of the immune response, including those features described hereinabove) cellular and humoral responses possible. In certain embodiments, this can include the use of heterologous prime-boost strategies, in which the compositions of this invention are used in combination with a composition comprising one or more of the following: immunogens derived from a pathogen or tumor, recombinant immunogens, naked nucleic acids, nucleic acids formulated with lipid-containing moieties, and viral vectors (including but not limited to alphavirus vectors, poxvirus vectors, adenoviral vectors, adeno-associated viral vectors, herpes virus vectors, vesicular stomatitis virus vectors, paramyxoviral vectors, parvovirus vectors, papovavirus vectors, retroviral vectors, lentivirus vectors).

A subject of this invention is any animal that is capable of producing an immune response against a coronavirus. A subject of this invention can also be any animal that is susceptible to infection by coronavirus and/or susceptible to diseases or disorders caused by coronavirus infection. A subject of this invention can be a mammal and in particular embodiments is a human, which can be an infant, a child, an adult, or an elderly adult. A “subject at risk of infection by a coronavirus” or a “subject at risk of coronavirus infection” is any subject who may be or has been exposed to a coronavirus.

In some embodiments, the chimeric coronavirus S protein may be administered once, twice, three times, four times, five times, six times, seven times, eight times, nine times, ten times or more, e.g., a primary (prime) administration and one or more secondary (boost) administrations. In some embodiments, the chimeric coronavirus S protein may be administered, for example, once a day, once every two days, once every three days, once every four days, once every five days, once every six days, once every seven days (once a week), once every two weeks, once every three weeks, once every four weeks, and/or once a month, etc., for multiple repetitions, e.g., twice a day, twice a week, twice a month, three times a day, three times a week, three times a month, etc. for one repetition, for two repetitions, for three repetitions, for four repetitions, for five repetitions, for six repetitions, or more. For example, in some embodiments, the chimeric coronavirus S protein may be administered every two weeks for two, three, or four or more repetitions. In some embodiments, the chimeric coronavirus S protein may be administered every three weeks for two, three, or four or more repetitions. In some embodiments, the chimeric coronavirus S protein may be administered every four weeks for two, three, or four or more repetitions.

In some embodiments, the chimeric coronavirus S protein(s) of the present invention administered in the one or more secondary administration (boost) may be a different chimeric coronavirus S protein from the chimeric coronavirus S protein(s) administered initially (the prime).

In some embodiments, the chimeric coronavirus S protein(s) of the present invention administered in the one or more secondary administration (boost) may be the same chimeric coronavirus S protein as the chimeric coronavirus S protein(s) administered initially (the prime).

In some embodiments, wherein an administration may comprise more than one chimeric coronavirus S protein of the present invention, the population of chimeric coronavirus S proteins administered in the one or more secondary administration (boost) may be the same population of chimeric coronavirus S proteins as the chimeric coronavirus S proteins administered initially (the prime).

In some embodiments, wherein an administration may comprise more than one chimeric coronavirus S protein of the present invention, one or more chimeric coronavirus S proteins of the population of chimeric coronavirus S proteins administered in the one or more secondary administration (boost) may be a different one or more chimeric coronavirus S proteins of the population of chimeric coronavirus S proteins as the chimeric coronavirus S proteins administered initially (the prime).

In some embodiments, a chimeric coronavirus S protein of the present invention (e.g., a chimeric coronavirus S protein and/or a nucleic acid molecule, vector, VRP, VLP, coronavirus particle, population and/or composition comprising the same) may be administered in a therapeutically effective amount. In some embodiments, a chimeric coronavirus S protein of the present invention may be administered in an amount of about 0.5 μg to about 250 μg or any value or range therein, e.g., about 0.5 μg, about 0.6 μg, about 0.7 μg, about 0.8 μg, about 0.9 μg, about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 110 μg, about 120 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg or any value or range therein. For example in some embodiments, a chimeric coronavirus S protein of the present invention may be administered in an amount of about 1 μg, about 5 μg, about 10 μg, about 75 μg, about 100 μg, about 150 μg, about 250 μg, or about 0.5 μg to about 15 μg, about 1 μg to about 200 μg, about 5 μg to about 250 μg, or about 2.5 μg to about 115 μg.

Compositions comprising two or more chimeric coronavirus S proteins of the present invention and/or isolated nucleic acid molecules encoding the same may comprise and/or be administered in an amount such that the two or more chimeric coronavirus S proteins are delivered at ratio of about 1:1, about 2:1, about 3:1, about 4:1, about 5:1, about 6:1, about 7:1 about 8:1, about 9:1, or about 10:1 or any value or range or range therein, e.g., about 1:1 ratio, e.g., about 1:1:1, about 1:1:1:1, about 1:1:1:1:1, about 1:1:11:1:1, about 1:1:1:1:1:11, about 1:1:1:1:1:1:1, about 1:1:1:1:1:1:1:1, about 1:1:1:1:1:1:1:1:1:1:1, about 1:1:1:1:1:1:1:1:1:1:1, about 1:1:1:1:1:1:1:1:1:1:1:1, about 1:1:1:1:1:1:1:1:1:1:1:1:1, 1:1:1:1:1:1:1:1:1:1:1:1:1:1, about 1:1:1:1:1:1:1:1:1:1:1:1:1:1:1, about 1:1:1:1:1:1:1:1:1:1:1:1:1:1:1:1, about 1:1:1:1:1:1:1:1:1:1:1:1:1:1:1:1:1, about 1:1:1:1:1:1:1:1:1:1:1:1:1:1:1:1:1:1, about 1:1:1:1:1:1:1:1:1:1:1:1:1:1:1:1:1:1:1, about 1:1:1:1:1:1:1:1:1:1:1:1:1:1:1:1:1:1:1:1, or about 2:1:1, about 1:2:1, about 1:1:2, about 1:1:10, about 1:10:1, or about 10:1:1, etc., or any value or range therein. For example, in some embodiments, a composition comprising two or more chimeric coronavirus S proteins of the present invention and/or an isolated nucleic acid molecule encoding the same may comprise each chimeric coronavirus S protein and/or nucleic acid molecule at a ratio for about 1:1, for a total amount of about 1 μg. In some embodiments, a composition comprising four or more chimeric coronavirus S proteins of the present invention and/or an isolated nucleic acid molecule encoding the same may comprise each chimeric coronavirus S protein and/or nucleic acid molecule at a ratio for about 1:1:1:1, for a total amount of about 1 μg. In some embodiments, a composition comprising twenty or more chimeric coronavirus S proteins of the present invention and/or an isolated nucleic acid molecule encoding the same may comprise each chimeric coronavirus S protein and/or nucleic acid molecule at a ratio for about 1:1:1:1:1:1:1:1:1:1:1:1:1:1:1:1:1:1:1:1, for a total amount of about 1 μg or more.

A nonlimiting example of an effective amount of a virus or virus particle (e.g., VRP) of this invention is from about 10⁴ to about 10¹⁰, preferably from about 10⁵ to about 10⁹, and in particular from about 10⁶ to about 10⁸ infectious units (IU, as measured by indirect immunofluorescence assay), or virus particles, per dose, which can be administered to a subject, depending upon the age, species and/or condition of the subject being treated. For subunit vaccines (e.g., purified antigens) a dose range of from about 1 to about 100 micrograms can be used. As would be well known to one of ordinary skill in the art, the optimal dosage would need to be determined for any given antigen or vaccine, e.g., according to the method of production and resulting immune response.

As one example, if the nucleic acid of this invention is delivered to the cells of a subject in an adenovirus vector, the dosage for administration of adenovirus to humans can range from about 10⁷ to 10⁹ plaque forming units (pfu) per injection, but can be as high as 10¹², 10¹⁵ and/or 10²⁰ pfu per injection. Ideally, a subject will receive a single injection. If additional injections are necessary, they can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired immunological response.

The exact amount of the nucleic acid or vector required will vary from subject to subject, depending on the species, age, weight and general condition of the subject, the particular nucleic acid or vector used, its mode of administration and the like. Thus, it is not possible to specify an exact amount for every nucleic acid or vector. However, an appropriate amount can be determined by one of ordinary skill in the art using only routine experimentation given the teachings herein.

For administration of serum or antibodies, as one nonlimiting example, a dosage range of from about 20 to about 40 international Units/Kilogram can be used, although it would be well understood that optimal dosage for administration to a subject of this invention needs to be determined, e.g., according to the method of production and resulting immune response.

In some embodiments, VEE replicon vectors can be used to express coronavirus structural genes in producing combination vaccines. Dendritic cells, which are professional antigen-presenting cells and potent inducers of T-cell responses to viral antigens, are preferred targets of VEE and VEE replicon particle infection, while SARS coronavirus targets the mucosal surfaces of the respiratory and gastrointestinal tract. As the VEE and coronavirus replicon RNAs synergistically interact, two-vector vaccine systems are feasible that may result in increased immunogenicity when compared with either vector alone. Combination prime-boost vaccines (e.g., DNA immunization and vaccinia virus vectors) have dramatically enhanced the immune response (notably cellular responses) against target papillomavirus and lentivirus antigens compared to single-immunization regimens (Chen et al. (2000) Vaccine 18:2015-2022; Gonzalo et al. (1999) Vaccine 17:887-892; Hanke et al. (1998) Vaccine 16:439-445; Pancholi et al. (2000) J. Infect. Dis. 182:18-27). Using different recombinant viral vectors (influenza and vaccinia) to prime and boost may also synergistically enhance the immune response, sometimes by an order of magnitude or more (Gonzalo, et al. (1999) Vaccine 17:887-892). Thus, the present invention also provides methods of combining different recombinant viral vectors (e.g., VEE and coronavirus) in prime boost protocols.

In the methods of this invention in which formation of an antigen/antibody complex is detected, a variety of assays can be employed for such detection. For example, various immunoassays can be used to detect antibodies or proteins (antigens) of this invention. Such immunoassays typically involve the measurement of antigen/antibody complex formation between a protein or peptide (i.e., an antigen) and its specific antibody.

The immunoassays of the invention can be either competitive or noncompetitive and both types of assays are well-known and well-developed in the art. In competitive binding assays, antigen or antibody competes with a detectably labeled antigen or antibody for specific binding to a capture site bound to a solid surface. The concentration of labeled antigen or antibody bound to the capture agent is inversely proportional to the amount of free antigen or antibody present in the sample.

Noncompetitive assays of this invention can be, for example, sandwich assays, in which, for example, the antigen is bound between two antibodies. One of the antibodies is used as a capture agent and is bound to a solid surface. The other antibody is labeled and is used to measure or detect the resultant antigen/antibody complex by e.g., visual or instrument means. A number of combinations of antibody and labeled antibody can be used, as are well known in the art. In some embodiments, the antigen/antibody complex can be detected by other proteins capable of specifically binding human immunoglobulin constant regions, such as protein A, protein L or protein G. These proteins are normal constituents of the cell walls of streptococcal bacteria. They exhibit a strong nonimmunogenic reactivity with immunoglobulin constant regions from a variety of species. (See, e.g., Kronval et al. J. Immunol. 111:1401-1406 (1973); Akerstrom et al. J. Immunol. 135:2589-2542 (1985)).

In some embodiments, the non-competitive assays need not be sandwich assays. For instance, the antibodies or antigens in the sample can be bound directly to the solid surface. The presence of antibodies or antigens in the sample can then be detected using labeled antigen or antibody, respectively.

In some embodiments, antibodies and/or proteins can be conjugated or otherwise linked or connected (e.g., covalently or noncovalently) to a solid support (e.g., bead, plate, slide, dish, membrane or well) in accordance with known techniques. Antibodies can also be conjugated or otherwise linked or connected to detectable groups such as radiolabels (e.g., ³⁵S, ¹²⁵I, ³²P, ¹³H, ¹⁴C, ¹³¹I), enzyme labels (e.g., horseradish peroxidase, alkaline phosphatase), gold beads, chemiluminescence labels, ligands (e.g., biotin) and/or fluorescence labels (e.g., fluorescein) in accordance with known techniques.

A variety of organic and inorganic polymers, both natural and synthetic can be used as the material for the solid surface. Nonlimiting examples of polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials that can be used include, but are not limited to, paper, glass, ceramic, metal, metalloids, semiconductive materials, cements, and the like. In addition, substances that form gels, such as proteins (e.g., gelatins), lipopolysaccharides, silicates, agarose, and polyacrylamides can be used. Polymers that form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes can be employed depending upon the nature of the system.

A variety of immunoassay systems can be used, including but not limited to, radio-immunoassays (RIA), enzyme-linked immunosorbent assays (ELISA) assays, enzyme immunoassays (EIA), “sandwich” assays, gel diffusion precipitation reactions, immunodiffusion assays, agglutination assays, immunofluorescence assays, fluorescence activated cell sorting (FACS) assays, immunohistochemical assays, protein A immunoassays, protein G immunoassays, protein L immunoassays, biotin/avidin assays, biotin/streptavidin assays, immunoelectrophoresis assays, precipitation/flocculation reactions, immunoblots (Western blot; dot/slot blot); immunodiffusion assays; liposome immunoassay, chemiluminescence assays, library screens, expression arrays, immunoprecipitation, competitive binding assays and immunohistochemical staining. These and other assays are described, among other places, in Hampton et al. (Serological Methods, a Laboratory Manual, APS Press, St Paul, Minn. (1990)) and Maddox et al. (J. Exp. Med. 158:1211-1216 (1993); the entire contents of which are incorporated herein by reference for teachings directed to immunoassays).

The methods of this invention can also be carried out using a variety of solid phase systems, such as described in U.S. Pat. No. 5,879,881, as well as in a dry strip lateral flow system (e.g., a “dipstick” system), such as described, for example, in U.S. Patent Publication No. 20030073147, the entire contents of each of which are incorporated by reference herein.

Embodiments of the present invention include monoclonal antibodies produced from B cells isolated from a subject of this invention that has produced an immune response against the chimeric coronavirus spike protein of this invention, wherein said monoclonal antibodies are specific to epitopes present on the chimeric coronavirus spike protein. Such monoclonal antibodies can be specific for an epitope in any of the first, second, third or fourth regions of the chimeric coronavirus spike protein of this invention as described herein.

The term “antibody” or “antibodies” as used herein refers to all types of immunoglobulins, including IgG, IgM, IgA, IgD, and IgE. The antibody can be monoclonal or polyclonal and can be of any species of origin, including, for example, mouse, rat, rabbit, horse, goat, sheep or human, or can be a chimeric or humanized antibody. See, e.g., Walker et al., Molec. Immunol. 26:403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980. The antibody can further be a single chain antibody or bispecific antibody. The antibody can also be humanized for administration to a human subject.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse et al., (1989) Science 254:1275-1281).

Monoclonal antibodies can be produced in a hybridoma cell line according to the technique of Kohler and Milstein, (1975) Nature 265:495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in bacterial cell such as E. coli by recombinant techniques known to those skilled in the art. See, e.g., W. Huse, (1989) Science 246:1275-81.

Antibodies can also be obtained by phage display techniques known in the art or by immunizing a heterologous host with a cell containing an epitope of interest.

In the manufacture of a pharmaceutical composition according to embodiments of the present invention, the composition of this invention is typically admixed with, inter alia, a pharmaceutically acceptable carrier. By “pharmaceutically acceptable carrier” is meant a carrier that is compatible with other ingredients in the pharmaceutical composition and that is not harmful or deleterious to the subject. A “pharmaceutically acceptable” component such as a salt, carrier, excipient or diluent of a composition according to the present invention is a component that (i) is compatible with the other ingredients of the composition in that it can be combined with the compositions of the present invention without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable components include, without limitation, any of the standard pharmaceutical carriers such as phosphate buffered saline solutions, water, emulsions such as oil/water emulsion, microemulsions and various types of wetting agents. A pharmaceutically acceptable carrier can comprise, consist essentially of or consist of one or more synthetic components (e.g., components that do not naturally occur in nature), as are known in the art.

The carrier may be a solid or a liquid, or both, and is preferably formulated with the composition of this invention as a unit-dose formulation. The pharmaceutical compositions are prepared by any of the well-known techniques of pharmacy including, but not limited to, admixing the components, optionally including one or more accessory ingredients. Exemplary pharmaceutically acceptable carriers include, but are not limited to, sterile pyrogen-free water and sterile pyrogen-free physiological saline solution. Such carriers can further include protein (e.g., serum albumin) and sugar (sucrose, sorbitol, glucose, etc.)

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### Examples

### Example 1: Generation of Chimeric Coronavirus Vaccine Antigens for Eliciting Neutralizing Antibody Responses Against Zoonotic and Pandemic Coronaviruses

This study was based on the hypothesis that chimeric coronavirus (CoV) particles may elicit better neutralizing antibody responses against diverse zoonotic and pandemic Group 2B CoVs as compared to a SARS-CoV-2 spike protein. Chimeric group 2B CoV antigens were designed with the goal to improve the protective efficacy of CoV vaccines against both zoonotic and pandemic CoVs that have the potential to emerge or that have previously emerged in humans.

The chimeric group 2B CoV vaccine antigens of this study were engineered to provide coverage against 1) SARS-CoV, which caused an epidemic in 2002-2003; 2) SARS-CoV-2, which has caused the COVID-19 pandemic; 3) HKU-3, which is a bat CoV capable of replication in human primary airway cells, suggesting it could emerge into a human population; and 4) SHC014, which is a bat CoV, and like HKU-3, can replicate in human primary airway cells and may be poised for human emergence. These chimeric spike vaccine particles comprise distinct modular parts of the spike protein that have been stitched together to provide maximum coverage against diverse Group 2B CoVs. Four chimeras developed in this study are described below.

Chimera #1 includes the N terminal domain (NTD) from HKU3, the receptor binding domain (RBD) from SARS-CoV, and the subunit 2 (S2) domain from SARS-CoV-2.

Chimera #2 includes the receptor binding domain (RBD) from SARS-CoV-2, the subunit 1 (S1) from SARS-CoV, and the subunit 2 (S2) domain from SARS-CoV.

Chimera #3 includes the receptor binding domain (RBD) from SARS-CoV, the subunit 1 (S1) from SARS-CoV-2, and the subunit 2 (S2) domain from SARS-CoV-2.

Chimera #4 includes the receptor binding domain (RBD) from SHC014, the subunit 1 (S1) from SARS-CoV-2, and the subunit 2 (S2) domain from SARS-CoV-2.

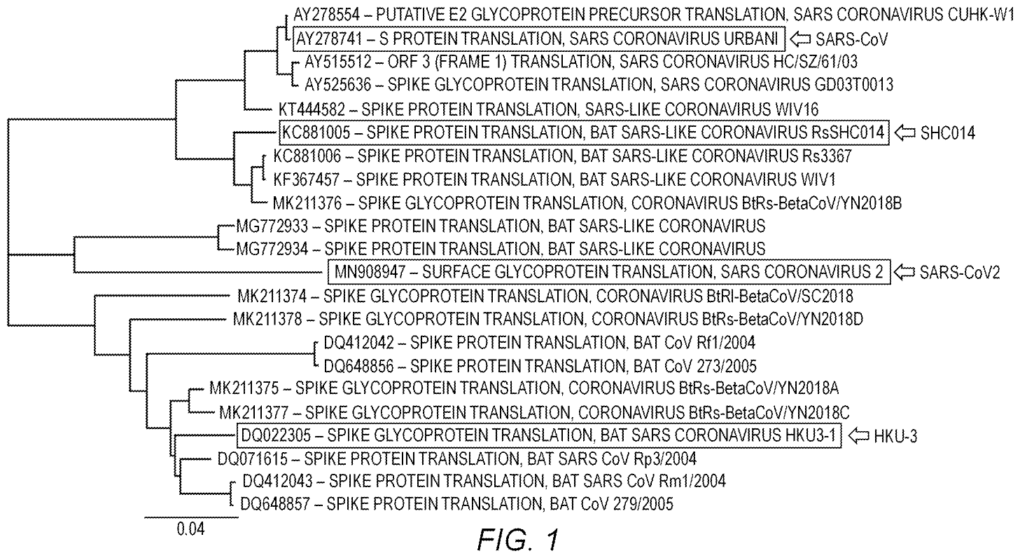

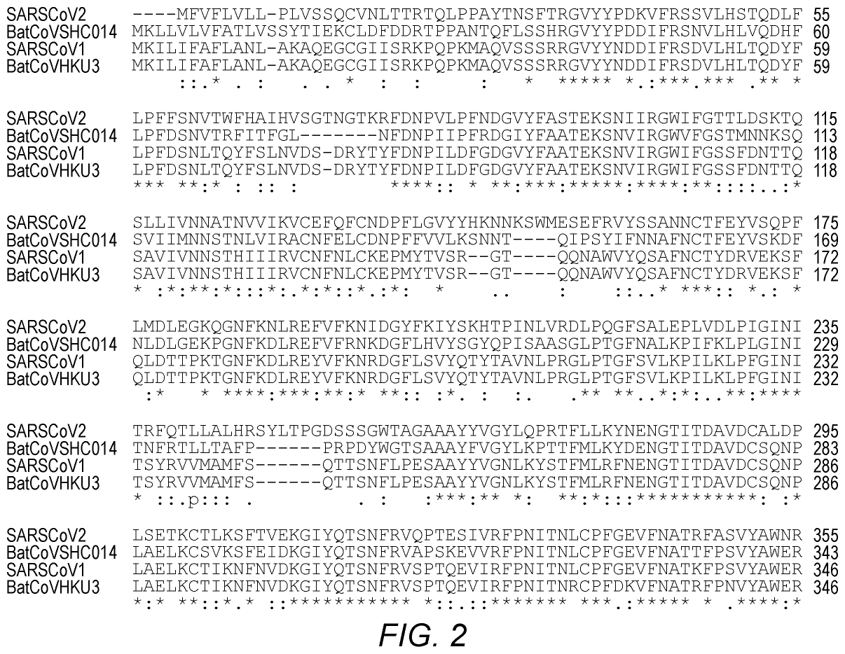

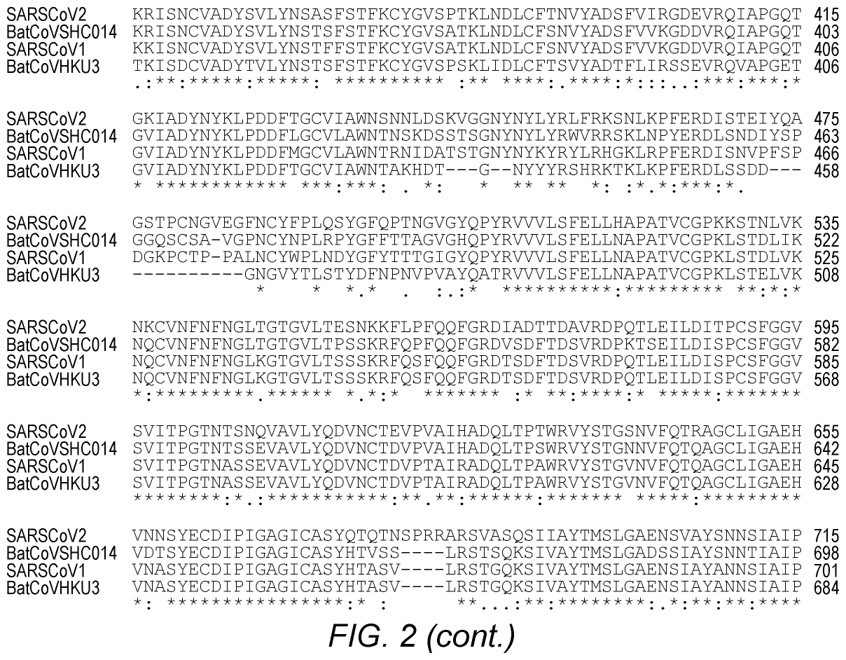

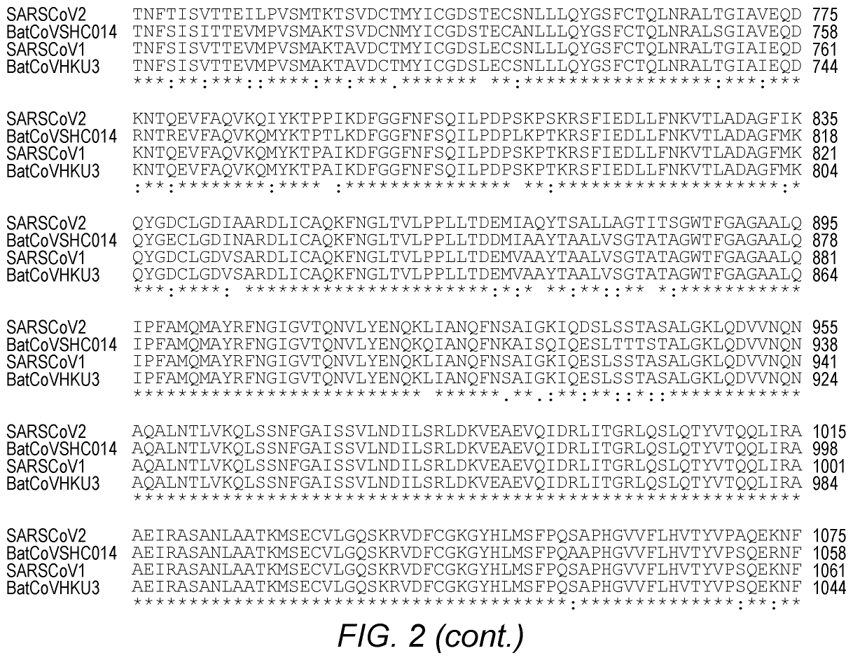

Thus, these chimeras comprise the antigenic portions that induce neutralizing antibodies and provide protection against major clusters of the Group 2B coronaviruses shown in FIG. 1. An alignment of the wildtype S protein amino acid sequences of the source CoVs (SARS-CoV-1, SARS-CoV-2, HKU3, and SHC014) is shown in FIGS. 2A-2B. The sequences of the generated chimeras are provided in the SEQUENCES portion of this application.

### Example 2: In Vivo Vaccination Using Chimeric Coronavirus Vaccine Antigens for Protection Against Zoonotic and Pandemic Coronaviruses

This series of chimeric spike proteins will be used alone or in combination to immunize mice with a prime-boost strategy comprising a vaccine prime and two boosts, two weeks apart. Different groups of mice will be immunized with a series of combinations of the chimeric spike vaccines, SARS-CoV-2 spike alone, and Zika virus envelope (E) protein, per the below mouse groupings.
Group 1: n=28 mice per vaccine group
   First vaccination: chimera 2/4;
      2 weeks
   Second vaccination: chimera 2/4;
      2 weeks
   Third vaccination: chimera 2/4.
   Mice with groups of n=7 will be challenged with the following viruses after receiving their second boost with the chimeric spike particles: SARS-CoV (n=7); SARS-CoV-2 (n=7); HKU3 (n=7); and SHC014 (n=7).
Group 2: n=28 mice per vaccine groups
   First vaccination: chimera 1;
      2 weeks
   Second vaccination: chimera 1;
      2 weeks
   Third vaccination: chimera 2.
   Mice with groups of n=7 will be challenged with the following viruses after receiving their second boost with the chimeric spike particles: SARS-CoV (n=7); SARS-CoV-2 (n=7); HKU3 (n=7); and SHC014 (n=7).
Group 3: n=28 mice per vaccine groups
   First vaccination: chimera 4;
      2 weeks
   Second vaccination: chimera 4;
      2 weeks
   Third vaccination: chimera 4.
   Mice with groups of n=7 will be challenged with the following viruses after receiving their second boost with the chimeric spike particles: SARS-CoV (n=7); SARS-CoV-2 (n=7); HKU3 (n=7); and SHC014 (n=7).
Group 4: n=28 mice per vaccine groups
   First vaccination: chimera 3;
      2 weeks
   Second vaccination: chimera 3;
      2 weeks
   Third vaccination: chimera 3.
   Mice with groups of n=7 will be challenged with the following viruses after receiving their second boost with the chimeric spike particles: SARS-CoV (n=7); SARS-CoV-2 (n=7); HKU3 (n=7); and SHC014 (n=7).
Group 5: n=28 mice per vaccine groups
   First vaccination: SARS2 wildtype spike;
      2 weeks
   Second vaccination: SARS2 wildtype spike;
      2 weeks
   Third vaccination: SARS2 wildtype spike.
   Mice with groups of n=7 will be challenged with the following viruses after receiving their second boost with the chimeric spike particles: SARS-CoV (n=7); SARS-CoV-2 (n=7); HKU3 (n=7); and SHC014 (n=7).
Group 6: n=28 mice per vaccine groups
   First vaccination: Zika virus E protein;
      2 weeks
   Second vaccination: Zika virus E protein;
      2 weeks
   Third vaccination: Zika virus E protein.
   Mice with groups of n=7 will be challenged with the following viruses after receiving their second boost with the chimeric spike particles: SARS-CoV (n=7); SARS-CoV-2 (n=7); HKU3 (n=7); and SHC014 (n=7).

Additional experiments comprising these groups will be carried out with intervals between vaccinations of about 3 weeks and/or about 4 weeks.

The chimeric particles will provide animals with better protection against diverse CoVs compared to mice that receive a monomorphic SARS-CoV-2 spike vaccine prime and two boosts. Group 1, Group 2, Group 3, and Group 4 mice will show better protection against lethal CoV challenge compared to Group 5 animals and Group 6 animals. Group 5 animals will only be protected against SARS-CoV-2, whereas all of the mice that receive the Zika envelope vaccine prime and boosts will become infected by all CoVs that the animals become exposed to. Thus, the chimeric spike CoV vaccines will provide improved vaccine protection, laying the groundwork for generating universal CoV vaccines against zoonotic and pandemic CoVs.

### Example 3: Chimeric Spike mRNA Vaccines Protect Against Sarbecovirus Challenge in Mice

Using chimeric spike designs, this study demonstrated protection against challenge from SARS-CoV, SARS-CoV-2, SARS-CoV-2 B.1.351, bat CoV (Bt-CoV) RsSHC014, and a heterologous Bt-CoV WIV-1 in vulnerable aged mice. Chimeric spike mRNAs induced high levels of broadly protective neutralizing antibodies against high-risk Sarbecoviruses. In contrast, SARS-CoV-2 mRNA vaccination not only showed a marked reduction in neutralizing titers against heterologous Sarbecoviruses, but SARS-CoV and WIV-1 challenge in mice resulted in breakthrough infections. Chimeric spike mRNA vaccines efficiently neutralized D614G, mink cluster five, the UK B.1.1.7., and South African B.1.351 variants of concern. Thus, multiplexed-chimeric spikes can prevent SARS-like zoonotic coronavirus infections with pandemic potential.

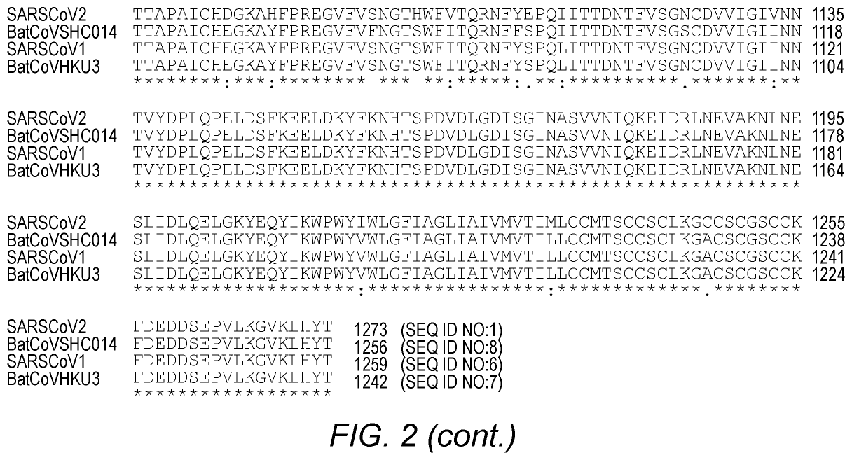

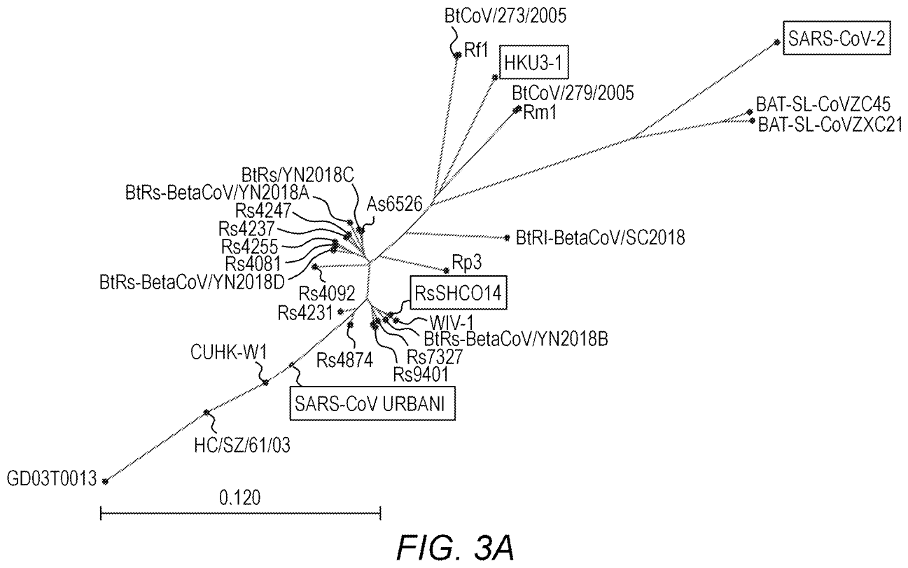

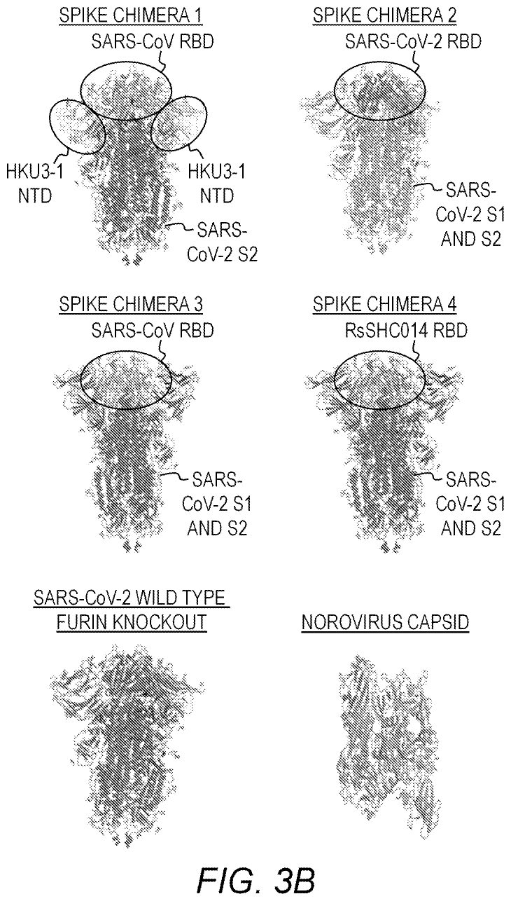

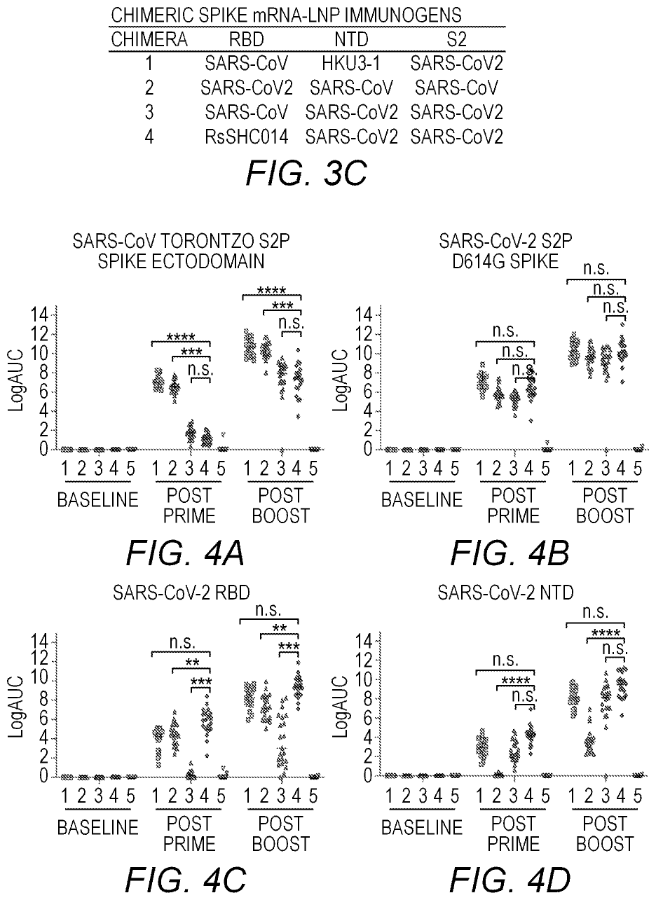

Design and expression of chimeric spike constructs to cover pandemic and zoonotic SARS-related coronaviruses: Sarbecoviruses exhibit considerable genetic diversity (FIG. 3A) and SARS-like bat CoVs (Bt-CoVs) are recognized threats to human health. This study designed four sets of chimeric spikes: Chimera 1 included the NTD from clade II Bt-CoV Hong Kong University 3-1 (HKU3-1), the clade I SARS-CoV RBD, and the clade III SARS-CoV-2 S2 (FIG. 3B). Chimera 2 included SARS-CoV-2 RBD and SARS-CoV NTD and S2 domains. Chimera 3 included the SARS-CoV RBD, and SARS-CoV-2 NTD and S2, while chimera 4 included the RsSHC014 RBD, and SARS-CoV-2 NTD and S2. The sequences of the generated chimeras are provided in the SEQUENCES portion of this application. A monovalent SARS-CoV-2 spike furin knock out (KO) vaccine, partially phenocopying the Moderna and Pfizer mRNA vaccines in human use, and a negative control norovirus GII capsid vaccine were also generated (FIGS. 3B and 3C).

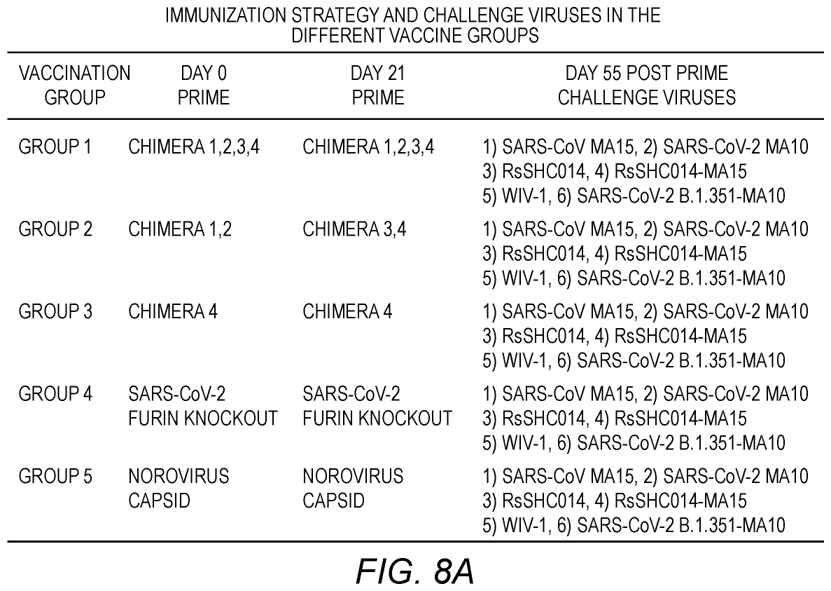

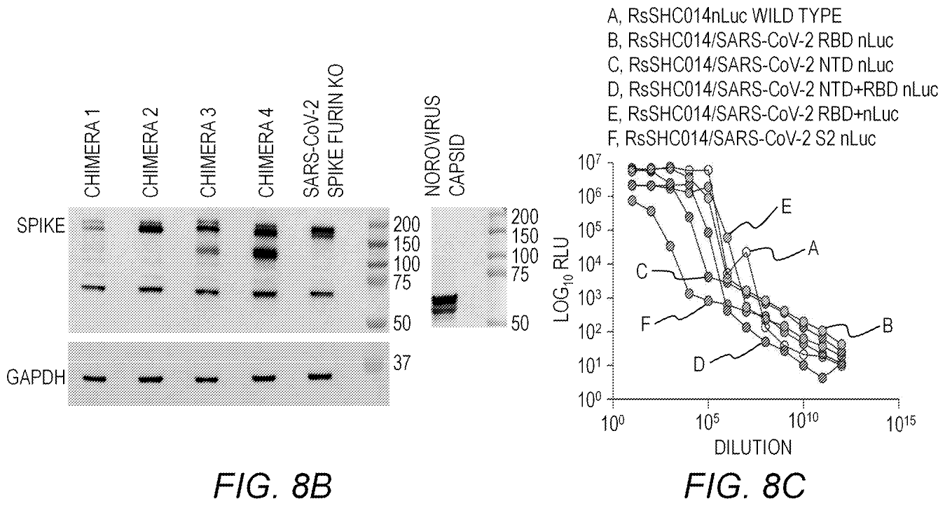

These chimeric spikes and control spikes were generated as lipid nanoparticle-encapsulated, nucleoside-modified mRNA vaccines with LNP adjuvants (mRNA-LNP), such as described in Laczkó et al. 2020 Immunity 53:724-732, incorporated herein by reference. The mRNA LNP stimulates robust T follicular helper cell activity, germinal center B cell responses, durable long-lived plasma cells, and memory B cell responses. Their chimeric spike expression was verified in HEK cells (FIG. 8B). To confirm that scrambled coronavirus spikes are biologically functional, several high titer recombinant live viruses of RsSHC014/SARS-CoV-2 S1, NTD, RBD and S2 domain chimeras were also designed and recovered that included deletions in non-essential, accessory ORF7&8 and that encoded nanoluciferase (FIG. 8C). SARS-CoV-2 ORF7 and 8 antagonize innate immune signaling pathways and deletions in these ORFs are associated with attenuated disease in humans.

Immunogenicity of mRNAs expressing chimeric spike constructs against coronaviruses: To determine if simultaneous immunization with mRNA-LNP expressing the chimeric spikes of diverse Sarbecoviruses was a feasible strategy to elicit broad binding and neutralizing antibodies, aged mice were immunized with the chimeric spikes formulated to induce cross-reactive responses against multiple divergent clade I-III Sarbecoviruses, a SARS-CoV-2 furin KO spike, and a GII.4 norovirus capsid negative control. Group 1 was primed and boosted with chimeric spikes 1, 2, 3, and 4 (FIG. 8A). Group 2 was primed with chimeric spikes 1 and 2 and boosted with chimeric spikes 3 and 4 (FIG. 8A). Group 3 was primed and boosted with chimeric spike 4 (FIG. 8A). Group 4 was primed and boosted with the monovalent SARS-CoV-2 furin knockout spike (FIG. 8A). Finally, group 5 was primed and boosted with a norovirus capsid GII.4 Sydney 2011 strain (FIG. 8A). Binding antibody responses were then examined by ELISA against a diverse panel of CoV spike proteins that included epidemic, pandemic, and zoonotic coronaviruses.

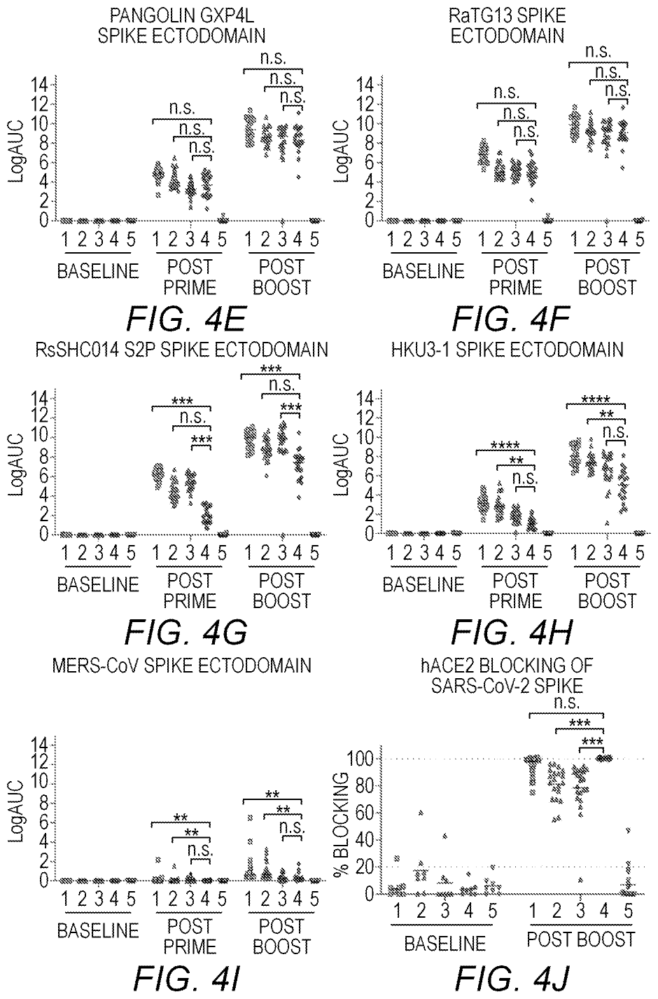

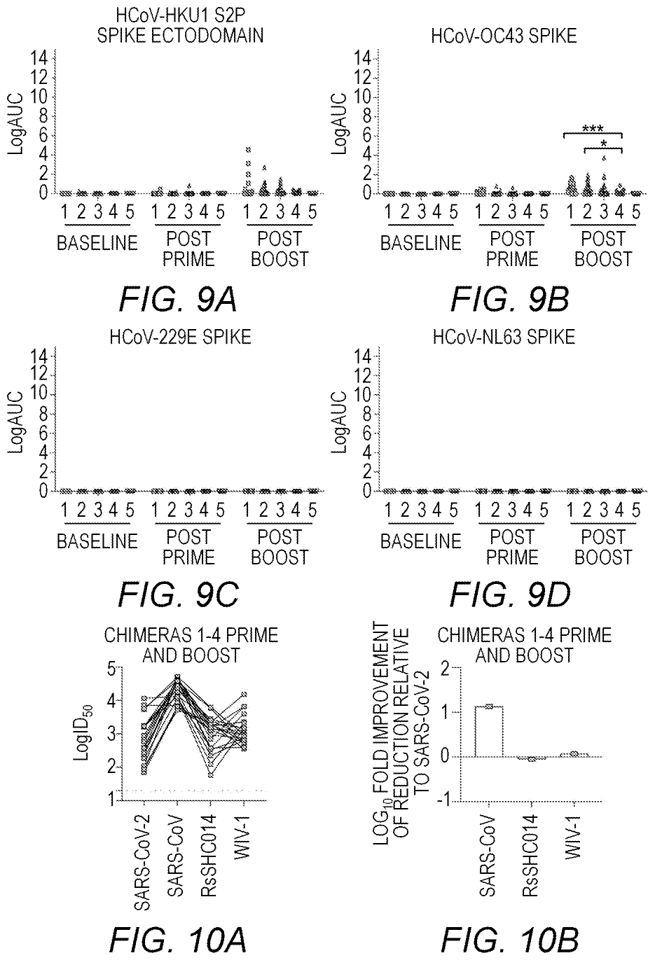

Mice in groups 1 and 2 generated the highest magnitude responses to SARS-CoV Toronto Canada isolate (Tor2), RsSHC014, and HKU3-1 spike compared to group 4 (FIGS. 4A, 4G, and 411). While mice in group 2 generated lower magnitude binding responses to both SARS-CoV-2 RBD (FIG. 4C) and SARS-CoV-2 NTD (FIG. 4D), mice in group 1 generated similar magnitude binding antibodies to SARS-CoV-2 D614G compared to mice immunized with the SARS-CoV-2 furin KO spike mRNA-LNP (FIG. 4B). Mice in groups 1 and 2 generated similar magnitude binding antibody responses against SARS-CoV-2 D614G, Pangolin GXP4L, and RaTG13 spikes (FIGS. 4B, 4E, and 4F) compared to mice from group 4. Mice in group 1 and group 4 elicited high magnitude levels of hACE2 blocking responses, as compared to groups 2 and 3 (FIG. 4J). As binding antibody responses post boost mirrored the trend of the post prime responses, it is likely that the second dose is boosting immunity to the vaccine antigens in the prime (FIGS. 4A-4J). Finally, we did not observe cross-binding antibodies against common-cold CoV spike antigens from HCoV-HKU1, HCoV-NL63, and HCoV-229E in most of the vaccine groups (FIGS. 9A-9D), but we did observe low binding levels against more distant group 2C MERS-CoV (FIG. 4I) and other Betacoronaviruses like group 2A HCoV-OC43 in vaccine groups 1 and 2 (FIG. 9B). These results suggest that chimeric spike vaccines elicit broader and higher magnitude binding responses against pandemic and bat SARS-like viruses compared to monovalent SARS-CoV-2 spike vaccines.

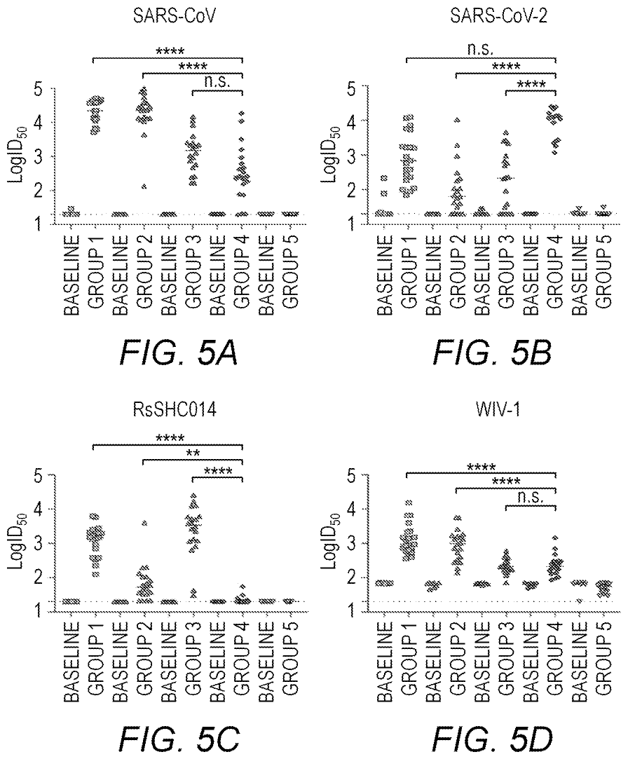

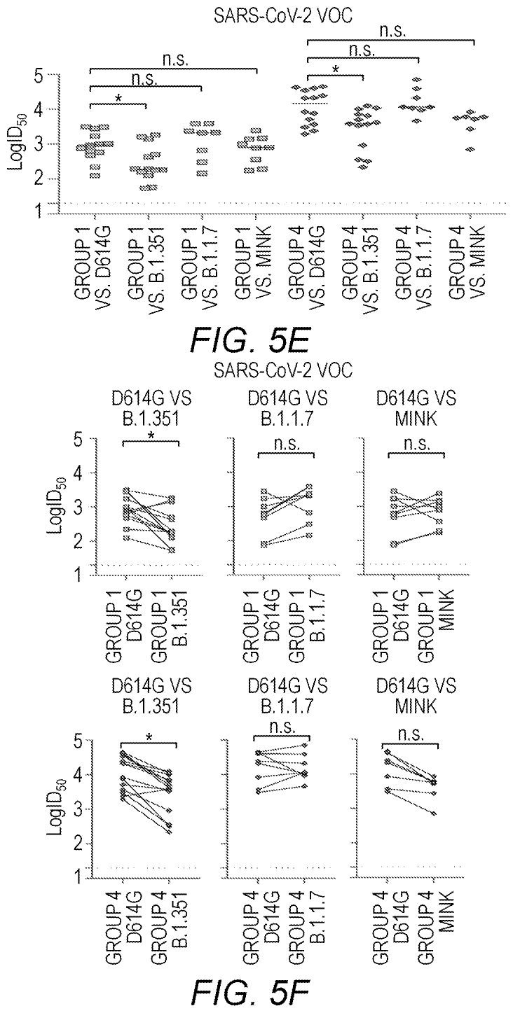

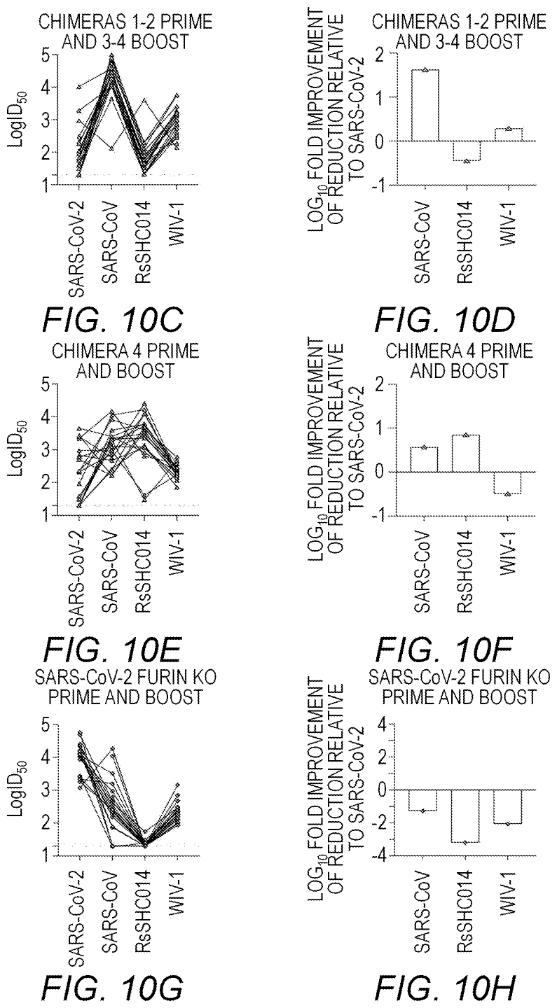

Neutralizing antibody responses against live Sarbecoviruses and variants of concern: Neutralizing antibody responses against SARS-CoV, Bt-CoV RsSHC014, Bt-CoV WIV-1, and SARS-CoV-2 and variants of concern were next examined using live viruses (FIGS. 5A-5D). Group 4 SARS-CoV-2 S mRNA vaccinated animals mounted a robust response against SARS-CoV-2, however responses against SARS-CoV, RsSHC014, and WIV-1 were 18-, >300- or 116-fold lower, respectively (FIGS. 5A-5D and FIGS. 10G-10H). In contrast, aged mice in group 2 showed a 42- and 2-fold increase in neutralizing titer against SARS-CoV and WIV1, and less than 1-fold decrease against RsSHC014 relative to SARS-CoV-2 neutralizing titers (FIGS. 5A-5D and FIGS. 10C-10D). Mice in group 3 elicited 3- and 7-fold higher neutralizing titers against SARS-CoV and RsSHC014 yet showed a 3-fold reduction in WIV-1 neutralizing titers relative to SARS-CoV-2 (FIGS. 5A-5D and FIGS. 10E-10F). Finally, mice in group 1 generated the most balanced and highest neutralizing titers that were 13- and 1.2-fold higher against SARS-CoV and WIV-1 and less than 1-fold lower against RsSHC014 relative to the SARS-CoV-2 neutralizing titers (FIGS. 5A-5D and FIGS. 10A-10B). The serum of mice from groups 1 and 4 neutralized the dominant D614G variant with similar potency as the wild type D614 non-predominant variant, and both groups had similar neutralizing antibody responses against the U.K. B.1.1.7 and the mink cluster 5 variant as compared to the D614G variant (FIGS. 5E and 5F). Despite the significant but small reduction in neutralizing activity against the B.1.351 variant of concern (VOC), we did not observe a complete ablation in neutralizing activity in either group. Mice from groups 1 and 2 elicited lower binding and neutralizing responses to SARS-CoV-2 compared to group 4 perhaps reflecting a lower amount of mRNA vaccine incorporated into multiplexed formulations, whereas the monovalent vaccines may drive a more focused B cell responses to SARS-CoV-2 whereas chimeric spike antigens lead to more breadth against distant Sarbecoviruses. Thus, both monovalent SARS-CoV-2 vaccines and multiplexed chimeric spikes elicit neutralizing antibodies against newly emerged SARS-CoV-2 variants and multiplexed chimeric spike vaccines outperform the monovalent SARS-CoV-2 vaccines in terms of breadth against multiclade Sarbecoviruses.

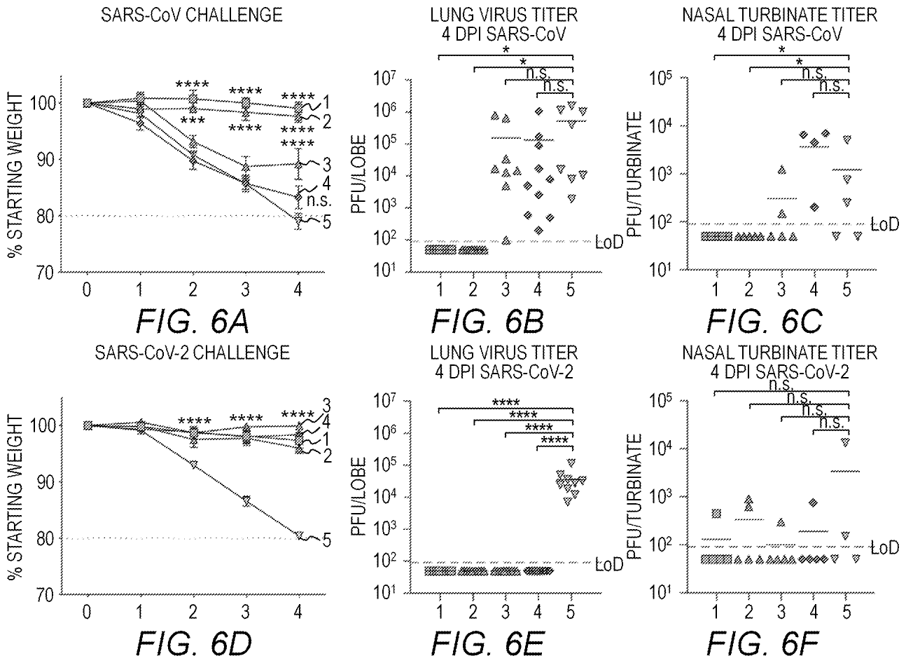

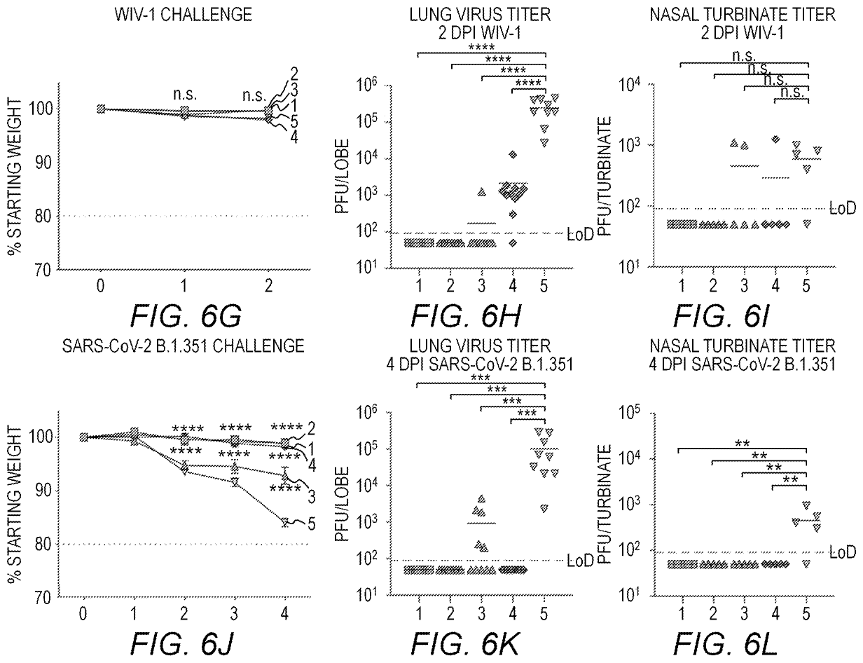

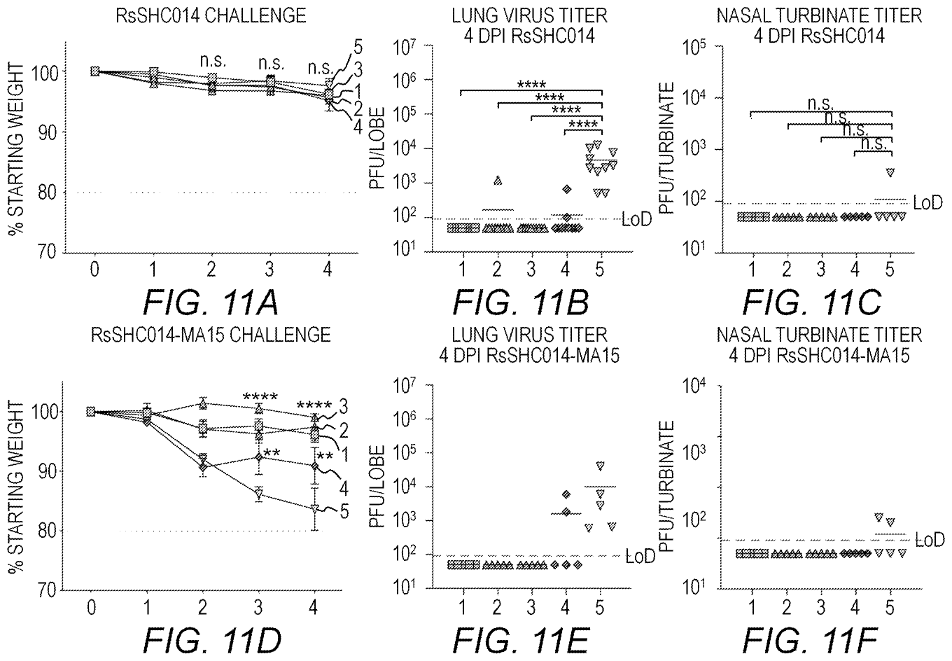

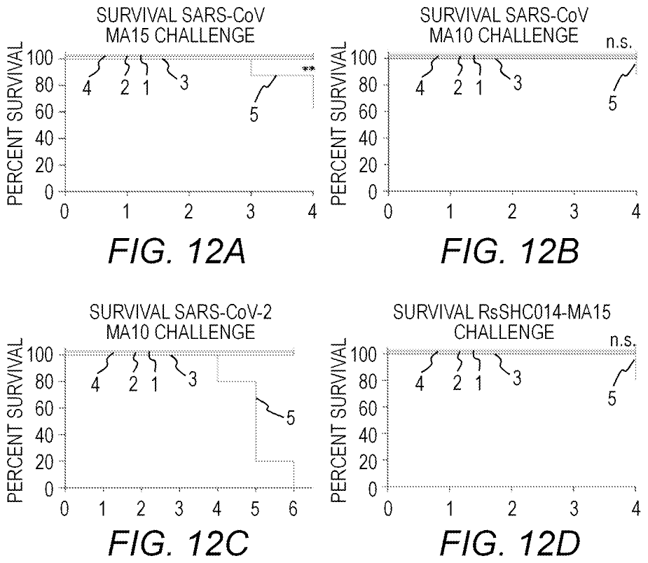

In vivo protection against heterologous Sarbecovirus challenge: To assess the ability of the mRNA-LNP vaccines to mediate protection against previously epidemic SARS-CoV, pandemic SARS-CoV-2, and Bt-CoVs, the different groups were challenged in mice and the mice observed for signs of clinical disease. Mice from group 1 or group 2 were completely protected from weight loss, lower, and upper airway virus replication as measured by infectious virus plaque assays following 2003 SARS-CoV mouse-adapted (MA15) challenge (FIGS. 6A, 6B and 6C). Similarly, these two vaccine groups were also protected against SARS-CoV-2 mouse-adapted (MA10) challenge. In contrast, group 3 showed some protection against SARS-CoV MA15 induced weight loss, but not against viral replication in the lung or nasal turbinates. Group 3 was fully protected against SARS-CoV-2 MA10 challenge. In contrast, group 5 vaccinated mice developed severe disease including mortality in both SARS-CoV MA15 and SARS-CoV-2 MA10 infections (FIGS. 12B and 12C). Monovalent SARS-CoV-2 mRNA vaccines were highly efficacious against SARS-CoV-2 MA10 challenge but failed to protect against SARS-CoV MA15-induced weight loss, and replication in the lower and upper respiratory tract (FIGS. 6A, 6B, and 6C), suggesting that SARS-CoV-2 mRNA-LNP vaccines are not likely to protect against future SARS-CoV emergence events. Mice from groups 1-4 were completely protected from weight loss and lower airway SARS-CoV-2 MA10 replication (FIGS. 6D, 6E, and 6F). Using both a Bt-CoV RsSHC014 full-length virus and a more virulent RsSHC014-MA15 chimera in mice (Menachery et al. 2015 Nat Med 21:1508-1513), protection was also demonstrated in groups 1-3 against RsSHC014 replication in the lung and nasal turbinates (FIGS. 11A-11F) but not in mice that received the SARS-CoV-2 mRNA vaccine. Group 5 control mice challenged with RsSHC014-MA15 developed disease including mortality (FIG. 12D). Group 3 mice, which received a SARS-CoV-2 NTD/RsSHC014 RBD/SARS-CoV-2 S2, were fully protected against both SARS-CoV-2 and RsSHC014 challenge whereas group 4 mice were not, demonstrating that a single NTD and RBD chimeric spike can protect against more than one virus compared to a monovalent spike.

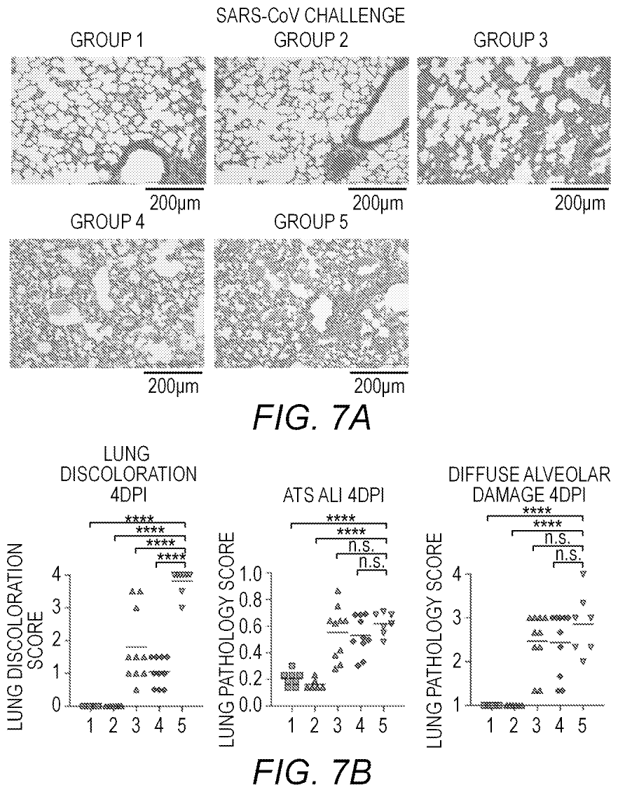

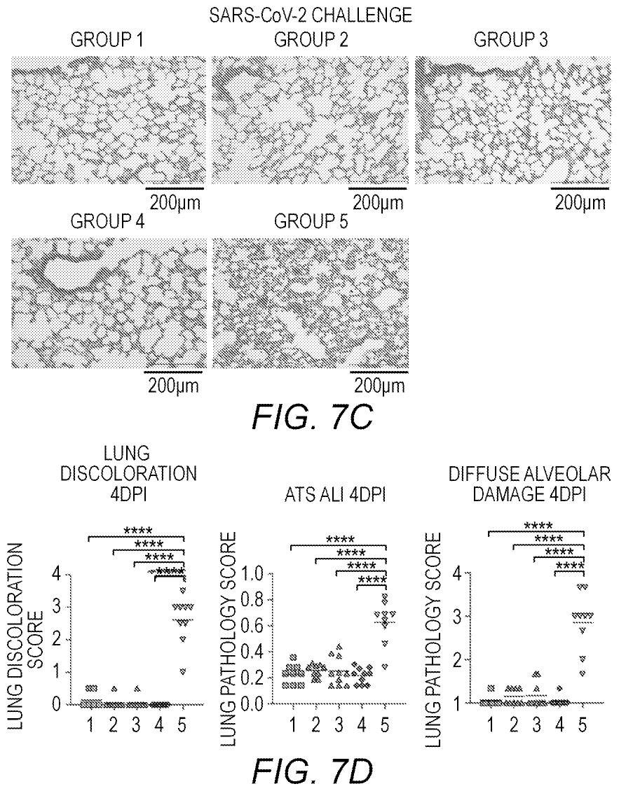

A heterologous challenge experiment was then performed with the bat pre-emergent WIV-1-CoV (Menachery et al. 2016 Proc Natl Acad Sci USA 113:3048-3053). Mice from groups 1 and 2 were fully protected against heterologous WIV-1 challenge whereas mice that received the SARS-CoV-2 mRNA vaccine had breakthrough replication in the lung (FIGS. 7G, 7H, and 7I). Mice were also challenged with a virulent form of SARS-CoV-2 VOC B.1.351, which contains deletions in the NTD and mutations in the RBD, and observed full protection in vaccine groups 1, 2, and 4 compared to controls, whereas breakthrough replication was observed in group 3, further underlining the importance of the NTD in vaccine-mediated protection (FIGS. 7J, 7K, and 7L). The reduced protection against the B.1.351 variant containing NTD deletions underlines that the NTD is a clear target of protective immunity and its inclusion in vaccination strategies, as opposed to RBD alone vaccines, may be required to achieve full protection. Moreover, the SARS-CoV-2 mRNA vaccine protected against SARS-CoV-2 B.1.351 challenge in aged mice despite a reduction in the neutralizing activity against this VOC.

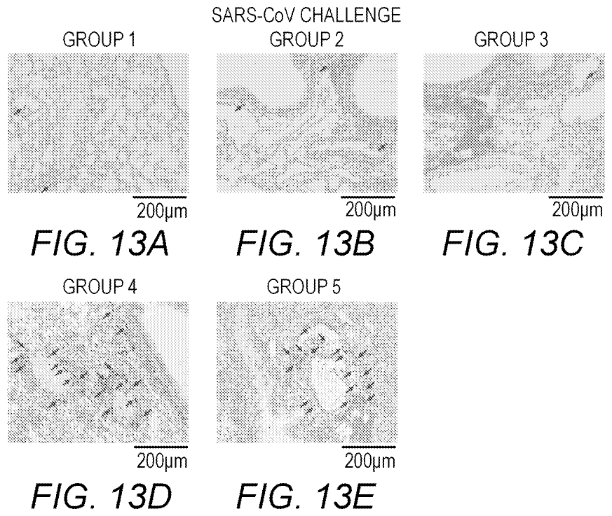

Lung pathology and cytokines in mRNA-LNP vaccinated mice challenged with epidemic and pandemic coronaviruses: Pathological features of acute lung injury (ALI) in mice were quantified according to methodology from the American Thoracic Society (ATS), and lung tissue sections were analyzed for diffuse alveolar damage (DAD), the pathological hallmark of ALI, such as described in Sheahan et al. (2020 Nat Commun 11:222) and Schmidt et al. (2018 PLoS Pathog 14:e1006810). Significant lung pathology was observed by both the ATS and DAD scoring tools in groups 4 and 5 vaccinated animals. In contrast, multiplexed chimeric spike vaccine formulations in groups 1 and 2 provided complete protection from lung pathology after SARS-CoV MA15 challenge (FIGS. 7A and 7B1). Mice immunized with the SARS-CoV-2 rnRNA vaccine that showed breakthrough infection with SARS-CoV MA15 developed similar lung inflammation as control vaccinated animals, potentially suggesting that future outbreaks of SARS-CoV may cause disease even in individuals vaccinated with SARS-CoV-2. Eosinophilic infiltrates have been previously observed in vaccinated, 2003 SARS-CoV challenged mice (Bolles et al. 2011 J Virol. 85:12201-12215). In this study, lung tissues in protected vs. infected animals with SARS-CoV MA15 were analyzed for eosinophilic infiltrates by immunohistochemistry (FIGS. 13A-13E). Groups 1 and 2 contained rare, scattered eosinophils in the interstitium. Group 3 showed bronchus-associated lymphoid tissue, while group 4 and group 5 contained frequent perivascular cuffs with prevalent eosinophils. In contrast, all groups challenged with SARS-CoV-2 MA10 were protected against lung pathology compared to the norovirus capsid-immunized control group, supporting the hypothesis that the SARS-CoV-2 NTD present in the chimeric spike from group 3 is sufficient for protection (FIGS. 7C and 7D).

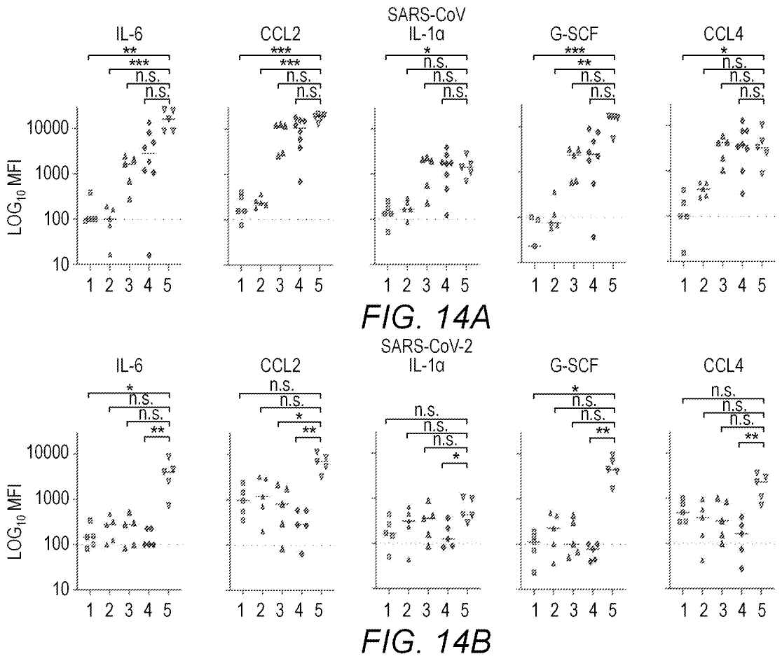

Lung proinflammatory cytokines and chemokines were measured in the different vaccination groups. Groups 1 and 2 had baseline levels of macrophage-activating cytokines and chemokines including, IL-6, CCL2, IL-1α, G-SCF, and CCL4, compared to group 5 following SARS-CoV MA15 challenge (FIG. 14A). Group 3 and group 4 showed high and indistinguishable levels of IL-6, CCL2, IL-1α, G-SCF, and CCL4 compared to group 5 mice following SARS-CoV MA15 challenge. Following SARS-CoV-2 MA10 challenge, group 4 and group 1 showed the lowest levels of IL-6, and G-SCF relative to group 5 controls (FIG. 14B), with significant reductions in CCL2, IL-1α, and CCL4 lung levels observed in groups 3 and 4 as compared to the group 5 control, despite full protection from both weight loss and lower airway viral replication.

Chimeric spike vaccine design and formulation. The chimeric spike vaccines of this study were designed with RBD and NTD swaps to increase coverage of epidemic (SARS-CoV), pandemic (SARS-CoV-2), and high-risk pre-emergent bat CoVs (bat SARS-like HKU3-1, and bat SARS-like RsSHC014). Chimeric and monovalent spike mRNA-LNP vaccines were designed based on SARS-CoV-2 spike (S) protein sequence (Wuhan-Hu-1, GenBank: MN908947.3), SARS-CoV (urbani GenBank: AY278741), bat SARS-like CoV HKU3-1 (GenBank: DQ022305), and Bat SARS-like RsSHC014 (GenBank: KC881005). Coding sequences of full-length SARS-CoV-2 furin knockout (RRAR furin cleavage site abolished between amino acid sequence positions 682-685 (Laczkó et al. 2020 Immunity 53:724-732), wherein the numbering corresponds to the reference amino acid sequence of wildtype SARS-CoV-2 spike (S) protein sequence Wuhan-Hu-1 GenBank Accession No. MN908947.3 (SEQ ID NO:1), the four chimeric spikes, and the norovirus capsid negative control were codon-optimized, synthesized and cloned into the mRNA production plasmid mRNAs were encapsulated with LNP. Briefly, mRNAs were transcribed to contain 101 nucleotide-long poly(A) tails, and modified with m1T-5′-triphosphate (TriLink #N-1081) instead of UTP and the in vitro transcribed mRNAs capped using the trinucleotide cap1 analog, CleanCap (TriLink #N-7413). mRNA was purified by cellulose (Sigma-Aldrich #11363-250G) purification. All mRNAs were analyzed by agarose gel electrophoresis and were stored at −20° C. Cellulose-purified m1Ψ-containing RNAs were encapsulated in proprietary LNPs containing adjuvant (Acuitas) using a self-assembly process wherein an ethanolic lipid mixture of ionizable cationic lipid, phosphatidylcholine, cholesterol and polyethylene glycol-lipid was rapidly mixed with an aqueous solution containing mRNA at acidic pH. The RNA-loaded particles were characterized and subsequently stored at −80° C. at a concentration of 1 mg/ml. The mean hydrodynamic diameter of these mRNA-LNP was about 80 nm with a polydispersity index of 0.02-0.06 and an encapsulation efficiency of about 95%.

Animals, immunizations, and challenge viruses. Eleven month old female BALB/c mice were used for all experiments. mRNA-LNP vaccines were kept frozen until right before vaccination. Mice were immunized with a total of 1 μg in the prime and boost. Briefly, chimeric vaccines were mixed at about 1:1 ratio for a total of 1 μg when more than one chimeric spike was used or 1 μg of a single spike diluted in sterile 1×PBS in a 50 μl volume and were given 25 μl intramuscularly in each hind leg. Equal amounts of vaccines were used to more compare the vaccine groups head-to-bead. Prime and boost immunizations were given three weeks apart. Three weeks post boost, mice were bled, sera was collected for analysis, and mice were moved into the BSL3 facility for challenge experiments. Animals were housed in groups of five and fed standard chow diets. Virus inoculations were performed under anesthesia and all efforts were made to minimize animal suffering. All mice were anesthetized and infected intranasally with 1×10⁴ PFU/ml of SARS-CoV MA15, 1×10⁴ PFU/ml of SARS-CoV-2 MA10, 1×10⁴ PFU/ml RsSHC014, 1×10⁴ PFU/ml RsSHC014-MA15, 1×10⁴ PFU/ml WIV-1, and 1×10⁴ PFU/ml SARS-CoV-2 B.1351-MA10. Mice were weighed daily and monitored for signs of clinical disease. Each challenge experiment encompassed 50 mice with 10 mice per vaccine group to obtain statistical power. Mouse vaccinations and challenge experiments were independently repeated twice to ensure reproducibility.

Measurement of mouse CoV spike binding antibodies by ELISA. Mouse serum samples from pre-immunization (pre-prime), 2 weeks post prime (pre-boost), and 3 weeks post boost were tested. A binding ELISA panel that included SARS-CoV spike protein Delta™, SARS-CoV-2 (2019-nCoV) spike protein (S1+S2 ECD, His tag) MERS-CoV, Coronavirus spike S1+S2 (Baculovirus-Insect cells, His), HKU1 (isolate N5) spike protein (S1-S2 ECD, His tag), OC43 spike protein (S1+S2 ECD, His Tag), 229E spike protein (S1+S2 ECD, His tag), Human coronavirus (HCoV-NL63) spike protein (S1+S2 ECD, His tag), Pangolin CoV_GXP4L_spikeEcto2P_3C8HtS2/293F, bat CoV RsSHC014_spikeEcto2P_3C8HtS2/293F, RaTG13_spikeEcto2P_3C8HtS2/293F, and bat CoV HKU3-1 spike were tested. Indirect binding ELISAs were conducted in 384 well ELISA plates coated with 2 μg/ml antigen in 0.1M sodium bicarbonate overnight at 4° C., washed and blocked with assay diluent (lx PBS containing 4% (w/v) whey protein/15% normal goat serum/0.5% Tween-20/0.05% sodium azide). Serum samples were incubated for 60 minutes in three-fold serial dilutions beginning at 1:30 followed by washing with PBS/0.1% Tween-20. HRP conjugated goat anti-mouse IgG secondary antibody (SouthernBiotech 1030-05) was diluted to 1:10,000 in assay diluent without azide, incubated for 1 hour at room temperature, washed and detected with 20 μl SureBlue Reserve (KPL 53-00-03) for 15 minutes. Reactions were stopped via the addition of 20 μl HCL stop solution. Plates were read at 450 nm. Area under the curve (AUC) measurements were determined from binding of serial dilutions.

ACE2 blocking ELISAs. Plates were coated with 2 μg/ml recombinant ACE2 protein, then washed and blocked with 3% BSA in PBS. While assay plates blocked, sera was diluted 1:25 in 1% BSA/0.05% Tween-20. Then SARS-CoV-2 spike protein was mixed with equal volumes of each sample at a final spike concentration equal to the EC₅₀ at which it binds to ACE2. The mixture was allowed to incubate at room temperature for 1 hour. Blocked assay plates were washed, and the serum-spike mixture was added to the assay plates for a period of 1 hour at room temperature. Plates were washed and Strep-Tactin HRP (IBA GmbH, Cat #2-1502-001) was added at a dilution of 1:5000 followed by TMB substrate. The extent to which antibodies were able to block the binding of spike protein to ACE2 was determined by comparing the OD of antibody samples at 450 nm to the OD of samples containing spike protein only without no antibody. The following formula was used to calculate percent blocking: (100-(OD sample/OD of spike only)*100).

Measurement of neutralizing antibodies against live viruses. Full-length SARS-CoV-2 Seattle, SARS-CoV-2 D614G, SARS-CoV-2 B.1.351, SARS-CoV-2 B.1.1.7, SARS-CoV-2 mink cluster 5, SARS-CoV, WIV-1, and RsSCH014 viruses were designed to express nanoluciferase (nLuc) and were recovered via reverse genetics. Virus titers were measured in Vero E6 USAMRIID cells, as defined by plaque forming units (PFU) per ml, in a 6-well plate format in quadruple biological replicates for accuracy. For the 96-well neutralization assay, Vero E6 USAMRIID cells were plated at 20,000 cells per well the day prior in clear bottom black walled plates. Cells were inspected to ensure confluency on the day of assay. Serum samples were tested at a starting dilution of 1:20 and were serially diluted 3-fold up to nine dilution spots. Serially diluted serum samples were mixed in equal volume with diluted virus. Antibody-virus and virus only mixtures were then incubated at 37° C. with 5% C02 for one hour. Following incubation, serially diluted sera and virus only controls were added in duplicate to the cells at 75 PFU at 37° C. with 5% C02. After 24 hours, cells were lysed, and luciferase activity was measured via Nano-Glo Luciferase Assay System (Promega) according to the manufacturer specifications. Luminescence was measured by a Spectramax M3 plate reader (Molecular Devices, San Jose, CA). Virus neutralization titers were defined as the sample dilution at which a 50% reduction in RLU was observed relative to the average of the virus control wells.

Eosinophilic lung infiltrates staining. To detect eosinophils, chromogenic immunohistochemistry (IHC) was performed on paraffin-embedded lung tissues that were sectioned at 4 microns. Lung tissues from vaccine groups 1-5 were analyzed for lung eosinophilic infiltration. N-8-10 lung tissues per group were analyzed. This IHC was carried out using the Leica Bond III Autostainer system. Slides were dewaxed in Bond Dewax solution (AR9222) and hydrated in Bond Wash solution (AR9590). Heat induced antigen retrieval was performed for 20 min at 100° C. in Bond-Epitope Retrieval solution 2, pH-9.0 (AR9640). After pretreatment, slides were incubated with an eosinophil peroxidase antibody (PA5-62200, Invitrogen) at 1:1000 for 1 hour followed with Novolink Polymer (RE7260-K) secondary. Antibody detection with 3,3′-diaminobenzidine (DAB) was performed using the Bond Intense R detection system (DS9263). Stained slides were dehydrated and coverslipped with Cytoseal 60 (8310-4, Thermo Fisher Scientific). Two positive controls (one with high and another with low eosinophil reactivity) and a negative control (no primary antibody) were included in all staining runs.

Lung pathology scoring. Lung discoloration is the gross manifestation of various processes of acute lung damage, including congestion, edema, hyperemia, inflammation, and protein exudation. A macroscopic scoring scheme was used to visually score mouse lungs at the time of harvest. Acute lung injury was quantified via two separate lung pathology scoring scales: Matute-Bello and Diffuse Alveolar Damage (DAD) scoring systems. Analyses and scoring were performed by a board certified veterinary pathologist who was blinded to the treatment groups. Lung pathology slides were read and scored at 600×total magnification.

The lung injury scoring system of the American Thoracic Society (Matute Bello) was used in order to help quantitate histological features of ALI observed in mouse models to relate this injury to human settings. In a blinded manner, three random fields of lung tissue were chosen and scored for the following: (A) neutrophils in the alveolar space (none=0, 1-5 cells=1, >5 cells=2), (B) neutrophils in the interstitial septa (none=0, 1-5 cells=1, >5 cells=2), (C) hyaline membranes (none=0, one membrane=1, >1 membrane=2), (D) proteinaceous debris in the air space (none=0, one instance=1, >1 instance=2), (E) alveolar septal thickening (<2× mock thickness=0, 2-4× mock thickness=1, >4× mock thickness=2). To obtain a lung injury score per field, A-E scores were put into the following formula score: [(20× A)+(14× B)+(7× C)+(7× D)+(2× E)]/100. This formula contains multipliers that assign varying levels of importance for each phenotype of the disease state. The scores for the three fields per mouse were averaged to obtain a final score ranging from 0 to and including 1. This lung histology scoring scale measures diffuse alveolar damage (DAD) (cellular sloughing, necrosis, hyaline membranes, etc.) Similar to the implementation of the ATS histology scoring scale, three random fields of lung tissue were scored for the following in a blinded manner: 1=absences of cellular sloughing and necrosis, 2=uncommon solitary cell sloughing and necrosis (1-2 foci/field), 3=multifocal (3+foci) cellular sloughing and necrosis with uncommon septal wall hyalinization, or 4=multifocal (>75% of field) cellular sloughing and necrosis with common and/or prominent hyaline membranes. The scores for the three fields per mouse were averaged to get a final DAD score per mouse. The microscope images were generated using an Olympus Bx43 light microscope and CellSense Entry v3.1 software.

Measurement of lung cytokines. Lung tissue was homogenized, spun down at 13,000 g, and supernatant was used to measure lung cytokines using Mouse Cytokine 23-plex Assay (BioRad). Briefly, 50 μl of lung homogenate supernatant was added to each well and the protocol was followed according to the manufacturer specifications. Plates were read using a MAGPIX multiplex reader (Luminex Corporation).

The foregoing is illustrative of the present invention, and is not to be construed as limiting thereof. The invention is defined by the following claims, with equivalents of the claims to be included therein.

SEQUENCES

SARS CoV WT S(NCBI Accession No. FJ211860) (SEQ ID NO: 6)
MKILIFAFLANLAKAQEGCGIISRKPQPKMAQVSSSRRGVYYND
DIFRSDVLHLTQDYFLPFDSNLTQYFSLNVDSDRYTYFDNPILDFGDGVYFAATEKSN
VIRGWIFGSSFDNTTQSAVIVNNSTHIIIRVCNFNLCKEPMYTVSRGTQQNAWVYQSA
FNCTYDRVEKSFQLDTTPKTGNFKDLREYVFKNRDGELSVYQTYTAVNLPRGLPTGES
VLKPILKLPFGINITSYRVVMAMFSQTTSNFLPESAAYYVGNLKYSTFMLRENENGTI
TDAVDCSQNPLAELKCTIKNFNVDKGIYQTSNFRVSPTQEVIRFPNITNLCPFGEVEN
ATKFPSVYAWERKKISNCVADYSVLYNSTFFSTFKCYGVSATKLNDLCFSNVYADSFV
VKGDDVRQIAPGQTGVIADYNYKLPDDEMGCVLAWNTRNIDATSTGNYNYKYRYLRHG
KLRPFERDISNVPFSPDGKPCTPPALNCYWPLNDYGFYTTTGIGYQPYRVVVLSFELL
NAPATVCGPKLSTDLVKNQCVNFNFNGLKGTGVLTSSSKRFQSFQQFGRDTSDFTDSV
RDPQTLEILDISPCSFGGVSVITPGTNASSEVAVLYQDVNCTDVPTAIRADQLTPAWR
VYSTGVNVFQTQAGCLIGAEHVNASYECDIPIGAGICASYHTASVLRSTGQKSIVAYT
MSLGAENSIAYANNSIAIPTNFSISVTTEVMPVSMAKTAVDCTMYICGDSLECSNLLL
QYGSFCTQLNRALTGIAIEQDKNTQEVFAQVKQMYKTPAIKDFGGFNFSQILPDPSKP
TKRSFIEDLLENKVTLADAGFMKQYGDCLGDVSARDLICAQKENGLTVLPPLLTDEMV
AAYTAALVSGTATAGWTFGAGAALQIPFAMQMAYRENGIGVTQNVLYENQKLIANQEN
SAIGKIQESLSSTASALGKLQDVVNQNAQALNTLVKQLSSNFGAISSVLNDILSRLDK
VEAEVQIDRLITGRLQSLQTYVTQQLIRAAEIRASANLAATKMSECVLGQSKRVDFCG
KGYHLMSFPQSAPHGVVFLHVTYVPSQEKNFTTAPAICHEGKAYFPREGVFVSNGTSW
FITQRNFYSPQLITTDNTFVSGNCDVVIGIINNTVYDPLQPELDSFKEELDKYFKNHT
SPDVDLGDISGINASVVNIQKEIDRLNEVAKNLNESLIDLQELGKYEQYIKWPWYVWL
GFIAGLIAIVMVTILLCCMTSCCSCLKGACSCGSCCKFDEDDSEPVLKGVKLHYT
SARS CoV2 WT S (NCBI Accession No. MN908947) (SEQ ID NO: 1)
MFVFLVLLPLVSSQCVNLTTRTQLPPAYTNSFTRGVYYPDKVER
SSVLHSTQDLFLPFFSNVTWFHAIHVSGTNGTKRFDNPVLPENDGVYFASTEKSNIIR
GWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQ
GFSALEPLVDLPIGINITRFQTLLALHRSYLTPGDSSSGWTAGAAAYYVGYLQPRTEL
LKYNENGTITDAVDCALDPLSETKCTLKSFTVEKGIYQTSNERVQPTESIVREPNITN
LCPFGEVENATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKVGGNYN
YLYRLFRKSNLKPFERDISTEIYQAGSTPCNGVEGENCYFPLQSYGFQPTNGVGYQPY
RVVVLSFELLHAPATVCGPKKSTNLVKNKCVNFNFNGLTGTGVLTESNKKELPFQQFG
RDIADTTDAVRDPQTLEILDITPCSFGGVSVITPGTNTSNQVAVLYQDVNCTEVPVAI
HADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFG
GFNFSQILPDPSKPSKRSFIEDLLENKVTLADAGFIKQYGDCLGDIAARDLICAQKEN
GLTVLPPLLTDEMIAQYTSALLAGTITSGWTFGAGAALQIPFAMQMAYRENGIGVTQN
VLYENQKLIANQFNSAIGKIQDSLSSTASALGKLQDVVNQNAQALNTLVKQLSSNFGA
ISSVLNDILSRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELD
SFKEELDKYFKNHTSPDVDLGDISGINASVVNIQKEIDRLNEVAKNLNESLIDLQELG
KYEQYIKWPWYIWLGFIAGLIAIVMVTIMLCCMTSCCSCLKGCCSCGSCCKFDEDDSE
PVLKGVKLHYT
Bat CoV HKU3 (NCBI Accession No. DQ022305) (SEQ ID NO: 7)
MKILIFAFLANLAKAQEGCGIISRKPQPKMAQVSSSRRGVYYNDDIFRSDVLHLTQDYFLPFDSNLTQYF
SLNVDSDRYTYFDNPILDFGDGVYFAATEKSNVIRGWIFGSSFDNTTQSAVIVNNSTHIIIRVCNENLCK
EPMYTVSRGTQQNAWVYQSAFNCTYDRVEKSFQLDTTPKTGNFKDLREYVFKNRDGFLSVYQTYTAVNLP
RGLPTGFSVLKPILKLPFGINITSYRVVMAMFSQTTSNFLPESAAYYVGNLKYSTFMLRFNENGTITDAV
DCSQNPLAELKCTIKNFNVDKGIYQTSNFRVSPTQEVIRFPNITNRCPFDKVENATRFPNVYAWERTKIS
DCVADYTVLYNSTSFSTFKCYGVSPSKLIDLCFTSVYADTFLIRSSEVRQVAPGETGVIADYNYKLPDDE
TGCVIAWNTAKHDTGNYYYRSHRKTKLKPFERDLSSDDGNGVYTLSTYDENPNVPVAYQATRVVVLSFEL
LNAPATVCGPKLSTELVKNQCVNFNFNGLKGTGVLTSSSKRFQSFQQFGRDTSDFTDSVRDPQTLEILDI
SPCSFGGVSVITPGTNASSEVAVLYQDVNCTDVPTAIRADQLTPAWRVYSTGVNVFQTQAGCLIGAEHVN
ASYECDIPIGAGICASYHTASVLRSTGQKSIVAYTMSLGAENSIAYANNSIAIPTNFSISVTTEVMPVSM
AKTAVDCTMYICGDSLECSNLLLQYGSFCTQLNRALTGIAIEQDKNTQEVFAQVKQMYKTPAIKDEGGEN
FSQILPDPSKPTKRSFIEDLLFNKVTLADAGFMKQYGDCLGDVSARDLICAQKENGLTVLPPLLTDEMVA
AYTAALVSGTATAGWTFGAGAALQIPFAMQMAYRENGIGVTQNVLYENQKLIANQFNSAIGKIQESLSST
ASALGKLQDVVNQNAQALNTLVKQLSSNFGAISSVLNDILSRLDKVEAEVQIDRLITGRLQSLQTYVTQQ
LIRAAEIRASANLAATKMSECVLGQSKRVDFCGKGYHLMSFPQSAPHGVVFLHVTYVPSQEKNFTTAPAI
CHEGKAYFPREGVFVSNGTSWFITQRNFYSPQLITTDNTFVSGNCDVVIGIINNTVYDPLQPELDSFKEE
LDKYFKNHTSPDVDLGDISGINASVVNIQKEIDRLNEVAKNLNESLIDLQELGKYEQYIKWPWYVWLGFI
AGLIAIVMVTILLCCMTSCCSCLKGACSCGSCCKFDEDDSEPVLKGVKLHYT
Bat CoV SHC014 (NCBI Accession No. KC881005) (SEQ ID NO: 8)
MKLLVLVFATLVSSYTIEKCLDFDDRTPPANTQFLSSHRGVYYPDDIFRSNVLHLVQDHFLPFDSNVTRF
ITFGLNFDNPIIPFRDGIYFAATEKSNVIRGWVFGSTMNNKSQSVIIMNNSTNLVIRACNFELCDNPFFV
VLKSNNTQIPSYIFNNAFNCTFEYVSKDENLDLGEKPGNFKDLREFVFRNKDGFLHVYSGYQPISAASGL
PTGFNALKPIFKLPLGINITNFRTLLTAFPPRPDYWGTSAAAYFVGYLKPTTFMLKYDENGTITDAVDCS
QNPLAELKCSVKSFEIDKGIYQTSNFRVAPSKEVVRFPNITNLCPFGEVENATTFPSVYAWERKRISNCV
ADYSVLYNSTSFSTFKCYGVSATKLNDLCFSNVYADSFVVKGDDVRQIAPGQTGVIADYNYKLPDDELGC
VLAWNTNSKDSSTSGNYNYLYRWVRRSKLNPYERDLSNDIYSPGGQSCSAVGPNCYNPLRPYGFFTTAGV
GHQPYRVVVLSFELLNAPATVCGPKLSTDLIKNQCVNFNFNGLTGTGVLTPSSKRFQPFQQFGRDVSDET
DSVRDPKTSEILDISPCSFGGVSVITPGTNTSSEVAVLYQDVNCTDVPVAIHADQLTPSWRVYSTGNNVF
QTQAGCLIGAEHVDTSYECDIPIGAGICASYHTVSSLRSTSQKSIVAYTMSLGADSSIAYSNNTIAIPTN
FSISITTEVMPVSMAKTSVDCNMYICGDSTECANLLLQYGSFCTQLNRALSGIAVEQDRNTREVFAQVKQ
MYKTPTLKDFGGFNFSQILPDPLKPTKRSFIEDLLENKVTLADAGFMKQYGECLGDINARDLICAQKENG
LTVLPPLLTDDMIAAYTAALVSGTATAGWTFGAGAALQIPFAMQMAYRENGIGVTQNVLYENQKQIANQF
NKAISQIQESLTTTSTALGKLQDVVNQNAQALNTLVKQLSSNFGAISSVLNDILSRLDKVEAEVQIDRLI
TGRLQSLQTYVTQQLIRAAEIRASANLAATKMSECVLGQSKRVDFCGKGYHLMSFPQAAPHGVVFLHVTY
VPSQERNFTTAPAICHEGKAYFPREGVFVENGTSWFITQRNFFSPQIITTDNTFVSGSCDVVIGIINNTV
YDPLQPELDSFKEELDKYFKNHTSPDVDLGDISGINASVVNIQKEIDRLNEVAKNLNESLIDLQELGKYE
QYIKWPWYVWLGFIAGLIAIVMVTILLCCMTSCCSCLKGACSCGSCCKFDEDDSEPVLKGVKLHYT
HKU3 NTD/SARS1 RBD/SARS2 S2 chimera (SEQ ID NO: 2)
MFVFLVLLPLVSSQCGIISRKPQPKMAQVSSSRRGVYYNDDIFRSDVLHLTQDYFLPF
DSNLTQYFSLNVDSDRYTYFDNPILDFGDGVYFAATEKSNVIRGWIFGSSFDNTTQSA
VIVNNSTHIIIRVCNFNLCKEPMYTVSRGTQQNAWVYQSAFNCTYDRVEKSFQLDTT
PKTGNFKDLREYVFKNRDGFLSVYQTYTAVNLPRGLPTGFSVLKPILKLPFGINITSY
RVVMAMFSQTTSNFLPESAAYYVGNLKYSTFMLRFNENGTITDAVDCSQNPLAELK
CTIKNFTVEKGIYQTSNFRVQPTESIVRFPNITNLCPFGEVFNATKFPSVYAWERKKIS
NCVADYSVLYNSTFFSTFKCYGVSATKLNDLCFSNVYADSFVVKGDDVRQIAPGQT
GVIADYNYKLPDDFMGCVLAWNTRNIDATSTGNYNYKYRYLRHGKLRPFERDISNV
PFSPDGKPCTPPALNCYWPLNDYGFYTTTGIGYQPYRVVVLSFELLNAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPRRARSVASQSIIAYTMSLGAEN
SVAYSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSFIEDLL
FNKVTLADAGFIKQYGDCLGDIAARDLICAQKFNGLTVLPPLLTDEMIAQYTSALLA
GTITSGWTFGAGAALQIPFAMQMAYRENGIGVTQNVLYENQKLIANQFNSAIGKIQD
SLSSTASALGKLQDVVNQNAQALNTLVKQLSSNFGAISSVLNDILSRLDKVEAEVQID
RLITGRLQSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPWYIWLGFIAG
LIAIVMVTIMLCCMTSCCSCLKGCCSCGSCCKFDEDDSEPVLKGVKLHYT
SARS2 RBD/SARS1 S1 and S2 chimera (SEQ ID NO: 3)
MFIFLLFLTLTSGSDLDRCTTFDDVQAPNYTQHTSSMRGVYYPDEIFRSDTLYLTQDL
FLPFYSNVTGFHTINHTFGNPVIPFKDGIYFAATEKSNVVRGWVFGSTMNNKSQSVIII
NNSTNVVIRACNFELCDNPFFAVSKPMGTQTHTMIFDNAFNCTFEYISDAFSLDVSEK
SGNFKHLREFVFKNKDGFLYVYKGYQPIDVVRDLPSGFNTLKPIFKLPLGINITNFRAI
LTAFSPAQDIWGTSAAAYFVGYLKPTTFMLKYDENGTITDAVDCSQNPLAELKCSVK
SFEIDKGIYQTSNFRVVPSGDVVRFPNITNLCPFGEVFNATRFASVYAWNRKRISNCV
ADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAG
STPCNGVEGFNCYFPLQSYGFQPTNGVGYQPYRVVVLSFELLHAPATVCGPKLSTDL
IKNQCVNFNFNGLTGTGVLTPSSKRFQPFQQFGRDVSDFTDSVRDPKTSEILDISPCSF
GGVSVITPGTNASSEVAVLYQDVNCTDVSTAIHADQLTPAWRIYSTGNNVFQTQAG
CLIGAEHVDTSYECDIPIGAGICASYHTVSLLRSTSQKSIVAYTMSLGADSSIAYSNNTI
AIPTNFSISITTEVMPVSMAKTSVDCNMYICGDSTECANLLLQYGSFCTQLNRALSGI
AAEQDRNTREVFAQVKQMYKTPTLKYFGGFNFSQILPDPLKPTKRSFIEDLLFNKVTL
ADAGFMKQYGECLGDINARDLICAQKFNGLTVLPPLLTDDMIAAYTAALVSGTATA
GWTFGAGAALQIPFAMQMAYRFNGIGVTQNVLYENQKQIANQFNKAISQIQESLTTT
STALGKLQDVVNQNAQALNTLVKQLSSNFGAISSVLNDILSRLDKVEAEVQIDRLITG
RLQSLQTYVTQQLIRAAEIRASANLAATKMSECVLGQSKRVDFCGKGYHLMSFPQA
APHGVVFLHVTYVPSQERNFTTAPAICHEGKAYFPREGVFVFNGTSWFITQRNFFSPQ
IITTDNTFVSGNCDVVIGIINNTVYDPLQPELDSFKEELDKYFKNHTSPDVDLGDISGIN
ASVVNIQKEIDRLNEVAKNLNESLIDLQELGKYEQYIKWPWYVWLGFIAGLIAIVMV
TILLCCMTSCCSCLKGACSCGSCCKFDEDDSEPVLKGVKLHYT
SARS1 RBD/SARS2 S1 and S2 chimera (SEQ ID NO: 4)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATKF
PSVYAWERKKISNCVADYSVLYNSTFFSTFKCYGVSATKLNDLCFSNVYADSFVVK
GDDVRQIAPGQTGVIADYNYKLPDDFMGCVLAWNTRNIDATSTGNYNYKYRYLRH
GKLRPFERDISNVPFSPDGKPCTPPALNCYWPLNDYGFYTTTGIGYQPYRVVVLSFEL
LNAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKG
VKLHYT
SCH014 RBD/SARS2 S1 and S2 chimera (SEQ ID NO: 5)
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVENATTF
PSVYAWERKRISNCVADYSVLYNSTSFSTFKCYGVSATKLNDLCFSNVYADSFVVK
GDDVRQIAPGQTGVIADYNYKLPDDFLGCVLAWNTNSKDSSTSGNYNYLYRWVRR
SKLNPYERDLSNDIYSPGGQSCSAVGPNCYNPLRPYGFFTTAGVGHQPYRVVVLSFE
LLNAPATVCGPKKSTNLVKNKCVNFNFNGLTGTGVLTESNKKFLPFQQFGRDIADTT
DAVRDPQTLEILDITPCSFGGVSVITPGTNTSNQVAVLYQDVNCTEVPVAIHADQLTP
TWRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYI
KWPWYIWLGFIAGLIAIVMVTIMLCCMTSCCSCLKGCCSCGSCCKFDEDDSEPVLKG
VKLHYT
Chimera 1 HKU3-1 NTD/SARS-COV RBD/SARS-COV-2 S2 (SEQ ID NO: 9)
MAISGVPVLGFFIIAVLMSAQESWAGIISRKPQPKMAQVSSSRRGVYYNDDIFRSDVL
HLTQDYFLPFDSNLTQYFSLNVDSDRYTYFDNPILDFGDGVYFAATEKSNVIRGWIFG
SSFDNTTQSAVIVNNSTHIIIRVCNFNLCKEPMYTVSRGTQQNAWVYQSAFNCTYDR
VEKSFQLDTTPKTGNFKDLREYVFKNRDGFLSVYQTYTAVNLPRGLPTGFSVLKPIL
KLPFGINITSYRVVMAMFSQTTSNFLPESAAYYVGNLKYSTFMLRFNENGTITDAVD
CSQNPLAELKCTIKNFTVEKGIYQTSNFRVQPTESIVRFPNITNLCPFGEVFNATKFPSV
YAWERKKISNCVADYSVLYNSTFFSTFKCYGVSATKLNDLCFSNVYADSFVVKGDD
VRQIAPGQTGVIADYNYKLPDDFMGCVLAWNTRNIDATSTGNYNYKYRYLRHGKL
RPFERDISNVPFSPDGKPCTPPALNCYWPLNDYGFYTTTGIGYQPYRVVVLSFELLNA
PATVCGPKKSTNLVKNKCVNFNFNGLTGTGVLTESNKKFLPFQQFGRDIADTTDAVR
DPQTLEILDITPCSFGGVSVITPGTNTSNQVAVLYQDVNCTEVPVAIHADQLTPTWRV
YSTGSNVFQTRAGCLIGAEHVNNSYECDIPIGAGICASYQTQTNSPRRARSVASQSIIA
YTMSLGAENSVAYSNNSIAIPTNFTISVTTEILPVSMTKTSVDCTMYICGDSTECSNLL
LQYGSFCTQLNRALTGIAVEQDKNTQEVFAQVKQIYKTPPIKDFGGFNFSQILPDPSK
PSKRSFIEDLLFNKVTLADAGFIKQYGDCLGDIAARDLICAQKFNGLTVLPPLLTDEMI
AQYTSALLAGTITSGWTFGAGAALQIPFAMQMAYRENGIGVTQNVLYENQKLIANQ
FNSAIGKIQDSLSSTASALGKLQDVVNQNAQALNTLVKQLSSNFGAISSVLNDILSRL
DKVEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
WYIWLGFIAGLIAIVMVTIMLCCMTSCCSCLKGCCSCGSCCKFDEDDSEPVLKGVKL
HYT
Chimera 2 SARS-COV-2 RBD/SARS-COV NTD and S2 (SEQ ID NO: 10)
MAISGVPVLGFFIIAVLMSAQESWASDLDRCTTFDDVQAPNYTQHTSSMRGVYYPDE
IFRSDTLYLTQDLFLPFYSNVTGFHTINHTFGNPVIPFKDGIYFAATEKSNVVRGWVF
GSTMNNKSQSVIIINNSTNVVIRACNFELCDNPFFAVSKPMGTQTHTMIFDNAFNCTF
EYISDAFSLDVSEKSGNFKHLREFVFKNKDGFLYVYKGYQPIDVVRDLPSGFNTLKPI
FKLPLGINITNFRAILTAFSPAQDIWGTSAAAYFVGYLKPTTFMLKYDENGTITDAVD
CSQNPLAELKCSVKSFEIDKGIYQTSNFRVVPSGDVVRFPNITNLCPFGEVENATRFAS
VYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYLYRLFRKSNLK
PFERDISTEIYQAGSTPCNGVEGENCYFPLQSYGFQPTNGVGYQPYRVVVLSFELLHA
PATVCGPKLSTDLIKNQCVNFNFNGLTGTGVLTPSSKRFQPFQQFGRDVSDFTDSVR
DPKTSEILDISPCSFGGVSVITPGTNASSEVAVLYQDVNCTDVSTAIHADQLTPAWRIY
STGNNVFQTQAGCLIGAEHVDTSYECDIPIGAGICASYHTVSLLRSTSQKSIVAYTMS
LGADSSIAYSNNTIAIPTNFSISITTEVMPVSMAKTSVDCNMYICGDSTECANLLLQYG
SFCTQLNRALSGIAAEQDRNTREVFAQVKQMYKTPTLKYFGGFNFSQILPDPLKPTK
RSFIEDLLFNKVTLADAGFMKQYGECLGDINARDLICAQKFNGLTVLPPLLTDDMIA
AYTAALVSGTATAGWTFGAGAALQIPFAMQMAYRENGIGVTQNVLYENQKQIANQ
FNKAISQIQESLTTTSTALGKLQDVVNQNAQALNTLVKQLSSNFGAISSVLNDILSRL
DKVEAEVQIDRLITGRLQSLQTYVTQQLIRAAEIRASANLAATKMSECVLGQSKRVD
FCGKGYHLMSFPQAAPHGVVFLHVTYVPSQERNFTTAPAICHEGKAYFPREGVFVFN
GTSWFITQRNFFSPQIITTDNTFVSGNCDVVIGIINNTVYDPLQPELDSFKEELDKYFK
NHTSPDVDLGDISGINASVVNIQKEIDRLNEVAKNLNESLIDLQELGKYEQYIKWPWY
VWLGFIAGLIAIVMVTILLCCMTSCCSCLKGACSCGSCCKFDEDDSEPVLKGVKLHYT
Chimera 3 SARS-COV RBD/SARS-COV-2 NTD and S2 (SEQ ID NO: 11)
MAISGVPVLGFFIIAVLMSAQESWAVNLTTRTQLPPAYTNSFTRGVYYPDKVFRSSVL
HSTQDLFLPFFSNVTWFHAIHVSGTNGTKRFDNPVLPFNDGVYFASTEKSNIIRGWIF
GTTLDSKTQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLVRDLPQGFS
ALEPLVDLPIGINITRFQTLLALHRSYLTPGDSSSGWTAGAAAYYVGYLQPRTFLLKY
NENGTITDAVDCALDPLSETKCTLKSFTVEKGIYQTSNFRVQPTESIVRFPNITNLCPF
GEVFNATKFPSVYAWERKKISNCVADYSVLYNSTFFSTFKCYGVSATKLNDLCFSNV
YADSFVVKGDDVRQIAPGQTGVIADYNYKLPDDFMGCVLAWNTRNIDATSTGNYN
YKYRYLRHGKLRPFERDISNVPFSPDGKPCTPPALNCYWPLNDYGFYTTTGIGYQPY
RVVVLSFELLNAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDVNCTEVPVA
IHADQLTPTWRVYSTGSNVFQTRAGCLIGAEHVNNSYECDIPIGAGICASYQTQTNSP
RRARSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFG
GFNFSQILPDPSKPSKRSFIEDLLFNKVTLADAGFIKQYGDCLGDIAARDLICAQKENG
LTVLPPLLTDEMIAQYTSALLAGTITSGWTFGAGAALQIPFAMQMAYRENGIGVTQN
VLYENQKLIANQFNSAIGKIQDSLSSTASALGKLQDVVNQNAQALNTLVKQLSSNFG
AISSVLNDILSRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNTVYDPLQPE
LDSFKEELDKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGSCCKFDED
DSEPVLKGVKLHYT
Chimera 4 RsSHC014 RBD/Remaining Spike SARS-COV-2 (SEQ ID NO: 12)
MAISGVPVLGFFIIAVLMSAQESWAVNLTTRTQLPPAYTNSFTRGVYYPDKVFRSSVL
HSTQDLFLPFFSNVTWFHAIHVSGTNGTKRFDNPVLPFNDGVYFASTEKSNIIRGWIF
GTTLDSKTQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLVRDLPQGFS
ALEPLVDLPIGINITRFQTLLALHRSYLTPGDSSSGWTAGAAAYYVGYLQPRTFLLKY
NENGTITDAVDCALDPLSETKCTLKSFTVEKGIYQTSNFRVQPTESIVRFPNITNLCPF
GEVENATTFPSVYAWERKRISNCVADYSVLYNSTSFSTFKCYGVSATKLNDLCFSNV
YADSFVVKGDDVRQIAPGQTGVIADYNYKLPDDFLGCVLAWNTNSKDSSTSGNYN
YLYRWVRRSKLNPYERDLSNDIYSPGGQSCSAVGPNCYNPLRPYGFFTTAGVGHQP
YRVVVLSFELLNAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQDVNCTE
VPVAIHADQLTPTWRVYSTGSNVFQTRAGCLIGAEHVNNSYECDIPIGAGICASYQTQ
TNSPRRARSVASQSIIAYTMSLGAENSVAYSNNSIAIPTNFTISVTTEILPVSMTKTSVD
CTMYICGDSTECSNLLLQYGSFCTQLNRALTGIAVEQDKNTQEVFAQVKQIYKTPPIK
DFGGFNFSQILPDPSKPSKRSFIEDLLFNKVTLADAGFIKQYGDCLGDIAARDLICAQK
FNGLTVLPPLLTDEMIAQYTSALLAGTITSGWTFGAGAALQIPFAMQMAYRFNGIGV
TQNVLYENQKLIANQFNSAIGKIQDSLSSTASALGKLQDVVNQNAQALNTLVKQLSS
NFGAISSVLNDILSRLDKVEAEVQIDRLITGRLQSLQTYVTQQLIRAAEIRASANLAAT
KMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLID
LQELGKYEQYIKWPWYIWLGFIAGLIAIVMVTIMLCCMTSCCSCLKGCCSCGSCCKF
DEDDSEPVLKGVKLHYT

1. A chimeric coronavirus S protein, comprising a coronavirus S protein backbone from a first coronavirus that comprises the following amino acid substitutions wherein the numbering is based on the reference amino acid sequence of SEQ ID NO:1:
a) a first region comprising amino acid residues 16-305 comprising a coronavirus S protein N-terminal domain (NTD) from a second coronavirus that is different from the first coronavirus; and/or
b) a second region comprising amino acid residues 330-521 comprising a coronavirus S protein receptor binding domain (RBD) of a third coronavirus that is different from the first coronavirus and/or second coronavirus.

2-3. (canceled)

4. The chimeric coronavirus S protein of claim 1, wherein the chimeric coronavirus S protein is derived from a subgroup 1a coronavirus, a subgroup 1b coronavirus, a subgroup 2a coronavirus, a subgroup 2b coronavirus, a subgroup 2c coronavirus, a subgroup 2d coronavirus and/or a subgroup 3 coronavirus.

5. The chimeric coronavirus S protein of claim 4, wherein the chimeric coronavirus S protein is derived from a subgroup 2b coronavirus.

6. The chimeric coronavirus S protein of claim 5, wherein said first coronavirus, second coronavirus and/or third coronavirus are from a subgroup 2b coronavirus selected from the group consisting of Bat SARS CoV (GenBank Accession No. FJ211859), SARS CoV (GenBank Accession No. FJ211860), BtSARS.HKU3.1 (GenBank Accession No. DQ022305), BtSARS.HKU3.2 (GenBank Accession No. DQ084199), BtSARS.HKU3.3 (GenBank Accession No. DQ084200), BtSARS.Rm1 (GenBank Accession No. DQ412043), BtCoV.279.2005 (GenBank Accession No. DQ648857), BtSARS.Rf1 (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCoV.CUHK-W1 (GenBank Accession No. AY278554), SARSCoV.GD01 (GenBank Accession No. AY278489), SARSCoV.HC.SZ.61.03 (GenBank Accession No. AY515512), SARSCoV.SZ16 (GenBank Accession No. AY304488), SARSCoV.Urbani (GenBank Accession No. AY278741), SARSCoV.civet010 (GenBank Accession No. AY572035), SARSCoV.MA.15 (GenBank Accession No. DQ497008), Rs SHC014 (GenBank® Accession No. KC881005), Rs3367 (GenBank® Accession No. KC881006), WiV1 S (GenBank® Accession No. KC881007), SARS CoV2 (GenBank Accession No. MN908947), and any combination thereof.

7. The chimeric coronavirus S protein of claim 1, wherein:
the first coronavirus is subgroup 2b coronavirus SARS CoV2 (GenBank Accession No. MN908947), the second coronavirus is subgroup 2b coronavirus BtSARS.HKU3.1 (GenBank Accession No. DQ022305), and the third coronavirus is subgroup 2b coronavirus SARSCoV.Urbani (GenBank Accession No. AY278741);
the first coronavirus is subgroup 2b coronavirus SARSCoV.Urbani (GenBank Accession No. AY278741), the second coronavirus is subgroup 2b coronavirus SARSCoV.Urbani (GenBank Accession No. AY278741), and the third coronavirus is subgroup 2b coronavirus SARS CoV2 (GenBank Accession No. MN908947):
the first coronavirus is subgroup 2b coronavirus SARS CoV2 (GenBank Accession No. MN908947), the second coronavirus is subgroup 2b coronavirus SARS CoV2 (GenBank Accession No. MN908947), and the third coronavirus is subgroup 2b coronavirus SARSCoV.Urbani (GenBank Accession No. AY278741); or
the first coronavirus is subgroup 2b coronavirus SARS CoV2 (GenBank Accession No. MN908947), the second coronavirus is subgroup 2b coronavirus SARS CoV2 (GenBank Accession No. MN908947), and the third coronavirus is subgroup 2b coronavirus Rs SHC014 (GenBank® Accession No. KC881005).

8. The chimeric coronavirus S protein of claim 7, comprising the following amino acid residues:
amino acid residues 16-1259 of SEQ ID NO:2;
amino acid residues 14-1256 of SEQ ID NO:3;
amino acid residues 16-1272 of SEQ ID NO:4; or
amino acid residues 16-1272 of SEQ ID NO:5.

9. The chimeric coronavirus S protein of claim 7, comprising the amino acid sequence of any one of SEQ ID NOs:2-5_or a sequence at least about 90% identical thereto.

10-18. (canceled)

19. An isolated nucleic acid molecule encoding the chimeric coronavirus S protein of claim 1.

20. A vector comprising the isolated nucleic acid molecule of claim 19.

21. The vector of claim 20, comprising at least two or more isolated nucleic acid molecules, each isolated nucleic acid molecule encoding a different chimeric S protein of claim 1.

22. The vector of claim 20, wherein the vector is a nanoparticle.

23. The vector of claim 22, wherein the nanoparticle comprises an mRNA-encapsulating lipid nanoparticle.

24. A Venezuelan equine encephalitis replicon particle (VRP) comprising the isolated nucleic acid molecule of claim 19.

25. A virus like particle (VLP) comprising the chimeric coronavirus S protein of claim 1 and a matrix protein of any virus that can form a VLP.

26. A coronavirus particle comprising the chimeric coronavirus S protein of claim 1.

27. (canceled)

28. A composition comprising the chimeric S protein of claim 1 in a pharmaceutically acceptable carrier.

29-32. (canceled)

33. A method of producing an immune response to a coronavirus in a subject, comprising administering to the subject an effective amount of the chimeric coronavirus S protein of claim 1, thereby producing an immune response to a coronavirus in the subject.

34. A method of treating a coronavirus infection in a subject in need thereof, comprising administering to the subject an effective amount of the chimeric coronavirus S protein of claim 1, thereby treating a coronavirus infection in the subject.

35. A method of preventing a disease or disorder caused by a coronavirus infection in a subject, comprising administering to the subject an effective amount of the chimeric coronavirus S protein of claim 1, thereby preventing a disease or disorder caused by a coronavirus infection in the subject.

36. A method of protecting a subject from the effects of coronavirus infection, comprising administering to the subject an effective amount of the chimeric coronavirus S protein of claim 1, thereby protecting the subject from the effects of coronavirus infection.

37-45. (canceled)
